(19) United States
(12) Patent Application Publication
(10) Pub. No.: US 2024/0181135 A1
(43) Pub. Date: Jun. 6, 2024

(54) BIOCHEMICAL ACTIVATION OF DYSFUNCTIONAL SKELETAL STEM CELLS FOR SKELETAL REGENERATION

(71) Applicant: The Board of Trustees of the Leland Stanford Junior University, Stanford, CA (US)

(72) Inventors: Charles K.F. Chan, Redwood City, CA (US); Michael T. Longaker, Atherton, CA (US); Thomas Ambrosi, Redwood City, CA (US); Owen Marecic, Redwood City, CA (US); Irving L. Weissman, Stanford, CA (US); Adrian Mcardle, Toronto, Ontario (CA)

(21) Appl. No.: 18/286,541

(22) PCT Filed: Apr. 26, 2022
(86) PCT No.: PCT/US2022/026305

§ 371 Date: Oct. 11, 2023

Related U.S. Application Data
(60) Provisional application No. 63/179,686, filed on Apr. 26, 2021.

(51) Int. Cl. A61L 27/52; A61L 27/26; A61L 27/54; C08L 1/02; C08L 5/08; C08L 67/04; C08L 89/06

(52) U.S. Cl. CPC A61L 27/52; A61L 27/26; A61L 27/54; C08L 1/02; C08L 5/08; C08L 67/04; C08L 89/06; A61L 2300/256; A61L 2300/604; A61L 2430/02

(57) ABSTRACT

Aged skeletal stem cells are targeted for reactivation by administration of a combination of a bone morphogenetic protein (BMP) and an inhibitor of CSF1, which combination of factors may be topically administered to a targeted skeletal site. In some embodiments the topical administration comprises placement of an implant, e.g. a matrix, gel, scaffold, etc. for localized delivery of the factor at the targeted skeletal site.

### CROSS REFERENCE TO RELATED APPLICATION

The present application claims the benefit of and priority to U.S. Provisional Patent Application No. 63/179,686, filed Apr. 26, 2021, the entire disclosure of which is hereby incorporated by reference in its entirety.

### STATEMENT REGARDING FEDERALLY SPONSORED RESEARCH

This invention was made with Government support under contract AG049958 awarded by the National Institutes of Health. The Government has certain rights in the invention.

### BACKGROUND

The progressive and gradual deterioration of an aging body is one of the most familiar yet resilient of medical challenges. Many of the underlying cellular, molecular, and genetic causes of aging, and the etiology of many age-related diseases remain poorly understood. Some aspects of aging are clearly rooted in cell-intrinsic alterations. On the other hand, studies of multi-cellular organisms like C. elegans have found that the initiation and progression of the aged phenotype is dependent on cell-extrinsic factors, such as altered systemic signaling. Many multi-cellular organisms contain tissue-resident, self-renewing stem/progenitor cells that consistently give rise to new cells for tissue building and regeneration and some species even evade aging because their somatic tissues are replenished continuously by their stem cell systems. Given that vertebrate animals also harbor stem cells in most of their somatic tissues, it is disappointing that we are nevertheless unable to escape the aging process. A possible explanation is that regenerative stem cells and their supportive niche environment are negatively affected by aging.

Stem cell aging has been characterized in detail in mouse hematopoietic stem cells (HSCs), which generate all the progenitors of mature blood lineages. In both mice and humans, there is an unexpected expansion in the frequency of HSCs during aging. Despite their greater abundance, aged HSCs are skewed in terms of their developmental output, generating elevated frequencies of myeloid lineages and significantly reduced numbers of lymphoid progenitors. These changes correspond to altered patterns of gene expression and regulation, as revealed by transcriptome and epigenetic analyses, but it is still unclear how these deviations relate to HSC aging. As HSCs normally reside in the marrow niches of adult bones, aging of HSCs and the hematopoietic system could be linked to aging of bones and skeletal tissues.

Age-related changes of bone and cartilage are well known and lead to conditions such as osteoarthritis and osteoporosis, representing major biomedical burdens in a rapidly aging global population. Here we utilize a new model system for understanding the origins of bone aging from the stem cell perspective. Progress in harnessing the clinical potential of bone-resident stem cells has been hampered by the lack of studies on purified bona fide skeletal stem cells. In our latest efforts, we have recently traced the origins of bone and cartilage tissue in mice and humans to a skeletal stem cell (SSC). In addition, downstream lineage cell populations derived from SSCs generate distinct types of supportive stromal cells that are capable of maintaining survival of specific kinds of hematopoietic progenitors, including HSCs. The analysis of skeletal aging from the stem cell perspective could provide important new insight into how aging occurs at the level of local stem cell niches, as well as systemic aspects of multi-organ physiological aging, eventually leading to new approaches to reverse its effects therapeutically.

Methods of therapeutically reversing the effects of aging on the regeneration of skeletal tissues is of consider medical and economic importance, and is addressed herein.

### SUMMARY

It is shown herein that deficient bone regeneration in aged mammals is due to a functional defect at the skeletal stem cell level. These stem cells have decreased bone-forming potential, and also give rise to high numbers of pro-inflammatory stroma that enhance bone resorption through actions on hematopoietic osteoclasts. Methods are provided for reversing this defect by the local application of a combinatorial treatment that re-activates aged SSCs and simultaneously abates crosstalk to hematopoietic cells favoring an inflammatory milieu. This treatment expands aged SSC pools, reduces osteoclast activity, and enhances bone healing.

In some embodiments, aged skeletal stem cells are targeted for reactivation by administration of an effective dose of a combination of a bone morphogenetic protein (BMP) and an inhibitor of CSF1. In some embodiments the combination of factors is topically administered to a targeted skeletal site. The targeted skeletal site may be a site of a skeletal injury, site of a skeletal implant, and the like. In some embodiments the topical administration comprises placement of an implant, e.g. a matrix, gel, scaffold, etc. for localized delivery of the factor at the targeted skeletal site. In some embodiments the targeted skeletal site is a fracture site. In some embodiments the reactivated SSC form bone at the targeted skeletal site. In some embodiments, additional factors are provided to skew the SSC to form cartilage at the targeted skeletal site, e.g. administration of a VEGF inhibitor. In some embodiments factors are provided in the absence of exogenous cells.

In some embodiments, a pharmaceutical composition is provided for the targeting of aged skeletal stem cells for reactivation. The active agents of the composition comprise or consist essentially of an effective dose of a bone morphogenetic protein (BMP) and an inhibitor of CSF1. The combination of factors may be formulated in an implant delivery device, e.g. a matrix, gel, scaffold, etc. for localized delivery of the factors at the targeted skeletal site. In some embodiments, additional factors are provided to skew the SSC to form cartilage at the targeted skeletal site, e.g. comprising a VEGF inhibitor. In some embodiments, additional factors are provided for activation of diabetic SSC, e.g. comprising a Hedgehog agent.

In some embodiments, the pharmaceutical composition is provided as an implant that is a biodegradable scaffold or matrix. In some embodiments a biodegradable matrix is a hydrogel. Various hydrogels are known and used in the art, and include, without limitation, hydrogels comprising polymers or co-polymers of poly-lactic acid, poly-glycolic acid, polyethylene glycol, and the like. The size of the implant will be appropriate for the bone lesion being treated.

In some embodiments, a pharmaceutical composition for topical administration to a targeted skeletal site comprises an effective dose of a combination of a BMP protein and a CSF1 inhibitor. In some embodiments the BMP protein is BMP2. In some embodiments the BMP protein is recombinant human BMP2. The dose of BMP2 provided in a unit dose in an implant for a mouse model may be from about 1 μg; about 2.5 μg; about 5 μg; about 7.5 μg; about 10 μg; about 12.5 μg; about 15 μg; and not more than about 25 μg; not more than about 20 μg; not more than about 15 μg. The corresponding unit dose for a human, e.g. an aged human, will be corresponding higher, and may be at least about 50 μg; at least about 100 μg; at least about 250 μg; at least about 500 μg; at least about 750 μg; at least about 1 mg; and not more than about 10 mg, not more than about 5 mg, not more than about 1 mg.

Preferably, an inhibitor of CSF1 is provided in the pharmaceutical composition for co-administration with the BMP protein. The dose of anti-CSF1 provided in a unit dose in an composition, e.g. an implant, for a mouse model will depend on the specific inhibitor but for an antibody may be, for example, from about 0.5 μg; about 1 μg; about 1.5 μg; about 2 μg; about 2.5 μg; and not more than about 5 μg; not more than about 4 μg; not more than about 2.5 μg. The corresponding unit dose for a human, e.g. an aged human, will be corresponding higher, and may be at least about 20 μg; at least about 25 μg; at least about 50 μg; at least about 75 μg; at least about 100 μg; and up to about 10 mg, up to about 5 mg, up to about 1 mg, up to about 500 μg, up to about 250 μg, up to about 150 μg. The dose of other anti-CSF1 agents, e.g. small molecule and antibody inhibitors of CSF1R, may be provided in a unit dose that is appropriately scaled to be comparable to these levels of anti-CSF1 antibody.

In some embodiments a method is provided for promoting bone healing in an aged individual diabetic patient, by administering a therapeutically effective amount of a hedgehog agent to the patient, in combination with a bone morphogenetic protein (BMP) and an inhibitor of CSF1. In some embodiments the hedgehog agent is a hedgehog protein. The hedgehog protein may be a human protein, or a variant or active fragment thereof. In some embodiments the hedgehog protein is human sonic hedgehog. In some embodiments the hedgehog protein is human indian hedgehog. In other embodiments the hedgehog agent is a small molecule agonist of Hedgehog signaling.

In some embodiments the pharmaceutical composition, e.g. an implant, is positioned on a targeted skeletal site so as to contact fully the skeletal lesion. Where a lesion is a fracture, for example, the implant may be “wrapped” around a long bone so that all surfaces of the lesion are in close proximity to the factors released by the implant.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIG. 1A-1O. Age-related bone loss coincides with altered skeletal stem cell function. (a) Schematic representation of the experimental setup investigating clonal activity in fractures of Actin-CreERT Rainbow mice in ‘2-mo’ (‘2-mo’) and ‘24-mo’ (‘24-mo’) mice (dpi: days post injury). (b-c) Representative gross images showing fracture callus at day-10 (top left), zoomed in outer callus region as Movat Pentachrome stain (bottom left), and fluorescent clones (right) in ‘2-mo’ and ‘24-mo’ mice. FC: fracture callus. (d) Quantification of clone size in ‘2-mo’ and ‘24-mo’ fracture calluses. Six distinct callus regions (5-19 clones per section) from two mice per age group were counted. (e) Flow cytometric quantification of SSC and BCSP frequency per femur in ‘2-mo’ and ‘24-mo’ mice under homeostatic conditions (n=7-15). (f-g) Prevalence of SSCs and BCSPs at different days after fracture injury in ‘2-mo’ and ‘24-mo’ animals-Fx: Fractured. (n=3-11). (h) Flow cytometric analysis of CD49f-expressing phenotypic SSCs and BCSPs under uninjured (uninj.) and fractured (fx; day-10) conditions in ‘2-mo’ and ‘24-mo’ animals (n=4). (i) Assessment of proliferative activity within SSCs and BCSPs at day-10 after fracture as measured by EdU-incorporation (n=6-7). (j) Assessment of apoptotic activity within SSCs and BCSPs at day-10 after fracture as measured by AnnexinV staining (n=3-4). (k) Flow cytometric analysis of lineage output of freshly isolated ‘2-mo’ and ‘24-mo’ SSCs cultured for six days (n=3). (l) In vitro osteogenic (top) and chondrogenic (bottom) capacity of ‘2-mo’ (2-mo) and ‘24-mo’ (24-mo) SSCs and BCSPs as determined by Alizarin Red S and Alcian Blue staining, respectively. Quantification of osteogenic (top right) and chondrogenic staining (bottom right) (n=3). (m) Renal capsule transplantation results of grafts excised 4 weeks after transplantation of GFP-labeled SSCs derived from day-10 fractures of ‘2-mo’ and ‘24-mo’ mice. Representative gross images of kidneys and zoomed in graft as brightfield and with GFP-signal (top three panels) each for ‘2-mo’ (left) and ‘24-mo’ (right) mouse derived cells. Sectioned grafts stained by Movat Pentachrome is displayed at the bottom. White and yellow arrows point at auto-fluorescent collagen sponge which is not part of the graft. (n) Representative μCT images (top) and quantification (bottom) of renal grafts derived from ‘2-mo’ or ‘24-mo’ SSCs (n=4-5). (o) TRAP-staining images (top) and quantification (bottom) for osteoclast surfaces in sections derived from renal grafts (n=4). All comparison of ‘2-mo’ vs. ‘24-mo’ groups by unpaired Student t-test adjusted for non-normality (Mann-Whitney test) and unequal variances (Welch's correction) where appropriate. Data shown as mean+SEM. Scale bars=50 μm.

FIG. 2A-20. The SSC lineage contributes to age-related hematopoietic lineage skewing. (a) Schematic representation of the experimental setup for parabiosis experiments. Isochronic ‘2-mo’ (IY), heterochronic pairs with one ‘2-mo’ (HY) and one ‘24-mo’ (HA) animal, and isochronic ‘24-mo’ (IA) pairs were generated and allowed to share blood circulation for four weeks before interventions. (b) Bone mineral density (BMD) four weeks after parabiosis surgery (n=4-6). (c) SSC and BCSP frequency as assessed by flow cytometry at four weeks of parabiosis (n=3-6). (d) BMD as assessed at day-10 after fracture of parabiosed mice (n=3-6). (e) In vitro osteogenic differentiation potential of SSCs isolated from parabiosed mice at day-10 after fracture showing representative staining at top and quantification at bottom (n=3). (f) In vitro chondrogenic differentiation potential of SSCs of the same cells (n=3). Statistical testing by one-way ANOVA analyses with Tukey's posthoc test for all comparisons. (g) Percentage of myeloid (My.) and lymphoid (Ly.) reconstitution from transplanted HSCs of parabionts into irradiated recipient mice (n=4). (h) Schematic of transplantation experiments. GFP-labeled HSCs from ‘2-mo’ mouse bone marrow was transplanted into lethally irradiated ‘2-mo’ or ‘24-mo’ mice. (i) Peripheral blood analysis at 6- and 12-weeks (wks) after hematopoietic reconstitution. Plots show overall chimerism as assessed by GFP+ cells, lymphoid (B- and T-cells), myeloid (Gr1+) fractions (n=5-6). Two-way ANOVA with Bonferroni posthoc test. (j) CD150/Slam expression in donor-derived GFP+Lin-cKit+Sca1+Flt3-Cd34− bone marrow HSCs. (k) Bone marrow analysis of donor-derived (GFP+) hematopoietic cell populations by flow cytometry at 12-weeks after transplantation (n=5-6). All comparison of ‘2-mo’ vs. ‘24-mo’ groups by unpaired Student t-test adjusted for non-normality (Mann-Whitney test) and unequal variances (Welch's correction) where appropriate. (l) Schematic of SSC-HSC co-culture experiments. (m) Flow cytometric analysis of lymphoid and myeloid cell types in 6-day co-cultures (n=3-4). One-way ANOVA with Tukey's posthoc test. (n) Peripheral blood analysis at 6- and 12-weeks (wks) after hematopoietic reconstitution with co-cultured hematopoietic cells. Plots show overall chimerism as assessed by GFP+ cells, lymphoid (B- and T-cells), myeloid (Gr1+) fractions (n=3-4). Two-way ANOVA with Bonferroni posthoc test. (o) Bone marrow analysis of co-cultured donor-derived (GFP+) hematopoietic cell populations by flow cytometry (n=3-4). One-way ANOVA with Tukey's posthoc test. Data is shown as mean+SEM. Scale bars=50 μm.

FIG. 3A-3Q. A pro-inflammatory aged skeletal lineage drives enhanced osteoclastic activity via Csf1. (a) UMAP plot showing Leiden cluster distribution of combined (postnatal day 3; ‘0-mo’), ‘2-mo’, and ‘24-mo’ single SSCs that underwent SmartSeq2 single cell RNA-sequencing. Chondro: Chondrogenic; Osteo: Osteogenic; pos: positive. (b) Clustering of same UMAP plot by age (bottom) showing distribution within Leiden clusters (top) of SmartSeq2-sequenced single cells by age. (c) Dotplot showing marker genes for each Leiden cluster. (d) Velocyto trajectory inference analysis in UMAP plot with cells labeled by Leiden cluster. (e) Heatmap of bulk microarray data for pro-inflammatory and pro-myeloid/-osteoclastic gene expression of purified skeletal lineage populations. Each cell population reflects expression of a pooled sample of three to five mice. (f) Quantification of the number of osteoclasts derived from ‘2-mo’ and ‘24-mo’ bone marrow in vitro (n=16-18, number per field of view, from three mice per age group). (g) Number of nuclei per derived osteoclast (n=14). (h) Representative brightfield images of in vitro derived osteoclasts. (i) Quantification of in vitro resorption activity of bone marrow derived osteoclasts from ‘2-mo’ and ‘24-mo’ mouse bone marrow (n=5 wells with cells from two different mice per age). (j) Representative brightfield images (right) in same experiment (n=5). Whisker plots show min to max. (k) Ligand (Csf1) and receptor (Csf1r) microarray bulk gene expression (in %) in the ‘2-mo’/′24-mo′ SSC-lineage and the hematopoietic lineage, respectively. If gene expression levels exceeded +25% a potential interaction was assumed and a connecting line between the two cell types was drawn. (l) Luminex protein data of Csf1 in supernatant of SSC and BCSP cultures of ‘2-mo’ and ‘24-mo’ mice (n=4). (m) Csf1 expression in RNA-sequencing data of ‘2-mo’, ‘12-mo’, and ‘24-mo’ SSCs of day-10 fracture calluses (n=3). One-tailed student t-test was used to compare ‘12-mo’ and ‘24-mo’ groups versus ‘2-mo’. (n) MicroCT analysis of Bone volume/Total volume (BV/TV) of day-10 fracture calluses locally treated with PBS or 5 μg of recombinant Csf1 (rCsf1; n=4). (o) Mechanical strength of uninjured femur bones from haploinsufficient Csf1-KO versus WT mice at 15-mo of age (n=4). (p) Movat Pentachrome staining of day-21 fracture callus tissue and (q) mechanical strength of the same calluses (n=4-6). All statistical testing by unpaired Student's t-test adjusted for non-normality (Mann-Whitney test) and unequal variances (Welch's correction) where appropriate. Data is shown as mean+SEM. Scale bars=150 μm.

FIG. 4A-4M. Combinatorial targeting of the ‘24-mo’ skeletogenic niche restores youthful fracture regeneration. (a) Schematic representation of experimental setup. Hydrogels with different combinations of recombinant factors were placed on freshly induced fractures of ‘24-mo’ mice and healing outcome assessed at day-10 and day-21. ‘2-mo’ old mice receiving hydrogels containing PBS were used as control. (b) Radiographic images of fractured femurs at day-10 (top) as well as μCT reconstructions of calluses at day 21 (middle) and their respective Movat Pentachrome (MP) staining of sections thereof are shown (bottom). (c) Callus index at day-10 after fracture induction and application of factors (Bmp2: 5 μg; Csf1low: 2 μg; Csf1high: 5 μg) (n=5-9). (d) Frequency of SSCs and BCSPs at day-10 (n=6-9). (e) Mechanical strength test (MST) at day-21 of fractured bones (n=6-13). (f) CFU-F capacity of SSCs isolated from ‘2-mo-PBS’, ‘24-mo-PBS’, and ‘24-mo-aCsf1^{low}+Bmp2’ treated fracture calluses at day-10 (n=5-6). (g) In vitro osteogenic capacity of SSCs isolated from ‘2-mo-PBS’, ‘24-mo-PBS’, and ‘24-mo-aCsf1^{low}+Bmp2’ treated fracture calluses at day-10 (n=4). Data is shown as mean+SEM. Statistical significance was calculated by Student's t-test between ‘2-mo PBS’ and each ‘24-mo’ groups and adjusted for non-normality (Mann-Whitney test) or unequal variances (Welch's test) where appropriate (n.s.: not significant). (h) Dotplot showing osteochondrogenic gene expression from 10× single cell RNA-sequencing experiment of ‘24-mo-PBS’ and ‘24-mo-aCsf1^{low}+Bmp2’ fracture calluses subset for non-hematopoietic cells. (i) Leiden clustering of 10× single cell RNA-sequencing experiment of ‘24-mo-PBS’ and ‘24-mo-aCsf1^{low}+Bmp2’ fracture calluses for cell fraction enriched for hematopoietic gene expression. (j) Same UMAP plot with cells labeled by treatment group. (k) Percentage of treatment group cell fraction per Leiden cluster. (l) Expression of Csf1r in hematopoietic cell fraction. (m) Dotplot showing early and late osteoclastic gene expression from 10× single cell RNA-sequencing experiment of ‘24-mo-PBS’ and ‘24-mo-aCsf1^{low}+Bmp2’ fracture calluses subset for the OC cluster. Scale bars=50 μm.

FIG. 5A-5I. Aging alters bone physiology and fracture healing in mice. (a) Representative Hematoxylin & Eosin (H&E) staining of proximal femurs from ‘2-mo’ (months), ‘middle-24-mo’, and ‘24-mo’ mice. (b) Three-dimensional μCT reconstruction of femoral bone mass in ‘2-mo’, ‘middle-24-mo’, and ‘24-mo’ mice. (c) Quantification of bone parameters by μCT measurements in the three age groups (n=3). (d) Bone formation rate (BFR) assessment by calcein labeling in ‘2-mo’ and ‘24-mo’ mice. MS: mineralizing surface; BS: bone surface; MAR: mineral apposition rate; BFR: bone formation rate (n=3). (e) Radiograph, μCT, and Movat Pentachrome staining images of fracture calluses at day-10 and day-21 after injury. (f) Callus index measurements at day-10 and day-21 after fracture in ‘2-mo’ and ‘24-mo’ femurs (n=3-5). (g) Mechanical strength test of fracture calluses at day-21 after fracture (n=8-10). (h) MicroCT images of fracture calluses from ‘2-mo’, middle-′24-mo′, and ‘24-mo’ mouse femurs at day-10 and day-21 after injury. (i) Quantification of fracture callus parameters by μCT measurements in the three age groups (n=3-6). Scale bars=150 μm. Scatter plot data shown as mean+SEM. Whisker plots show min to max. All comparison of groups against ‘2-mo’ by one-tailed, unpaired Student's t-test adjusted with non-normality (Mann-Whitney test) or unequal variances (Welch's test) where appropriate.

FIG. 6A-6F. Phenotypic SSCs are present in ‘24-mo’ mice. (a) The mouse skeletal stem cell lineage. A self-renewing skeletal stem cell (SSC) gives rise to a multi-potent bone-cartilage-stromal progenitor (BCSP) cell which is the precursor for committed cartilage, bone, and stromal lineages. (b) Schematic of experimental strategy to analyze intrinsic characteristics of highly purified ‘2-mo’ and ‘24-mo’ SSC lineage cells. (c) FACS gating strategy for the isolation of mouse SSC lineage cells. Representative FACS profiles for ‘2-mo’ and ‘24-mo’ animals are shown. (d) CD200 expression of SSC gated cells in ‘2-mo’ (blue) and ‘24-mo’ (red) mice. Fluorescence-minus-one (FMO) control shown in grey. (e) Flow cytometric quantification of Thy1+ and 6C3+ downstream cell population frequency in ‘2-mo’ and ‘24-mo’ mice in response to fracture at day-10 after injury (n=4). (f) Flow cytometric analysis of lineage output of freshly isolated ‘2-mo’ and ‘24-mo’ BCSPs cultured for six days (n=3). Scale bars=50 μm. Data shown as mean+SEM. Comparison of ‘2-mo’ and ‘24-mo’ age groups by unpaired Student's t-test adjusted for non-normality (Mann-Whitney test) and unequal variances (Welch's correction) where appropriate.

FIG. 7A-7F. SSCs/BCSPs display reduced functionality in vitro and in vivo. (a) Fibroblast colony forming unit (CFU-F) ability of ‘2-mo’ and ‘24-mo’ SSC-derived cell populations of long bones (n=6-10, three independent experiments). Two-way ANOVA with Bonferroni's posthoc test. (b) SSC and BCSP derived colony size of cells derived from uninjured, and day-10 fractured bones (n=7-120). Statistical testing between age groups by unpaired Student's t-test or Mann-Whitney test for non-normality. (c) Representative images of colonies stained by Crystal Violet. (d) In vitro adipogenic capacity of ‘2-mo’ and ‘24-mo’ SSCs and BCSPs as determined by Oil Red O staining (representative of n=3 biological replicates). (e) Renal capsule transplantation results of grafts excised 4 weeks after transplantation of GFP-labeled SSCs derived from uninjured long bones of ‘2-mo’ and ‘24-mo’ mice. Representative gross images of kidneys and zoomed in graft as brightfield and with GFP-signal (top four panels) each for ‘2-mo’ (left) and ‘24-mo’ (right) mouse derived cells. Sectioned grafts stained by Movat Pentachrome is displayed at the bottom. White and yellow arrows point at auto-fluorescent collagen sponge which is not part of the graft. (f) Same renal capsule experiment and results for BCSPs isolated from uninjured long bones or day-10 femoral fractures of ‘2-mo’ and ‘24-mo’ mice. Data shown as mean+SEM.

FIG. 8A-8S. Exposure to a young circulation does not rejuvenate the SSC lineage. (a) Results of FACS-analysis of blood in GFP+ and GFP-parabionts two weeks after conjoining to demonstrate shared circulation (n=3). (b) Thy1+ and 6C3+ frequency as assessed by flow cytometry at four weeks of parabiosis (n=3-6). (c) Callus index (highest width of callus divided by bone shaft width next to fracture) for parabiosed mice at day-10 (n=5-9) and day-21 (n=3-5) after fracture injury. Statistical testing by two-way ANOVA with Bonferroni posthoc test. (d) Representative gross images of femoral fracture calluses of parabiont groups. (e) SSC lineage frequencies as assessed by flow cytometry at day-10 after fracture (Fx) in parabionts (n=3-6). Statistical testing by one-way ANOVA analyses with Tukey's posthoc test for all comparisons. (f) Microarray-based inflammatory gene expression levels of purified SSCs from HA and HY mice. (g) Blood serum concentration of Rankl in circulation of 4-week parabionts (n=4). (h) Blood serum concentration of CTX1 in circulation of 4-week parabionts (n=2). (i) Representative images of TRAP staining of fracture calluses of parabionts. (j) Quantification of TRAP staining in fracture calluses of parabionts (n=3-4). Statistical testing by one-way ANOVA analyses with Tukey's posthoc test for all comparisons. (k) Schematic of experimental approach transplanting freshly isolated HSCs from fetal liver or ‘24-mo’ mice into either ‘2-mo’ or ‘24-mo’ lethally irradiated mice. (l) BMD in ‘2-mo’ and ‘24-mo’ lethally irradiated mice transplanted with fetal liver HSCs or HSCs from ‘24-mo’ mice eight weeks after hematopoietic reconstitution (n=5-6). (m) Callus Index of recipient mice at day-14 after fracture induced at eight-week time point after transplantation (n=4-5). (n) Representative FACS-gating strategy for myeloid (Gr1+) and lymphoid (B- and T-cells) cells in peripheral blood after hematopoietic reconstitution with GFP-donor HSCs (gated from Ter119⁻, alive cells). (0) Representative bone marrow FACS-gating strategy of GFP+ donor-derived cells for hematopoietic lineage tree populations. (p) Bone marrow analysis of donor-derived (GFP+) HSCs, MPP1, and MPP2 cell populations by flow cytometry (n=5-6). (q) Representative TRAP-staining and GFP-fluorescence images (same section) from day-10 fracture calluses of 2-mo and 24-mo mice reconstituted with GFP-labeled 2-mo HSCs. (r) Quantification of total area of TRAP⁺GFP⁺ regions in sections of fracture calluses of three mice per age group (n=3). (s) Bone marrow analysis of co-cultured donor-derived (GFP+) HSCs, MPP1, and MPP2 cell populations by flow cytometry (n=3-4). Data shown as mean+SEM. All comparison of ‘2-mo’ vs. ‘24-mo’ groups by unpaired Student t-test adjusted for non-normality (Mann-Whitney test) and unequal variances (Welch's correction) where appropriate.

FIG. 9A-9M. Distinct transcriptomic signatures in SSCs of different ages. (a) Heatmap of top 150 differentially expressed genes in each age group by Leiden clusters. (b) Gene count per single cell as violin plot grouped by age (left) and in UMAP plot. Statistical testing by Mann-Whitney test. (c) Heatmap showing expression of apoptosis related genes in single cell data grouped by age. (d) Heatmap showing expression of senescence associated genes in single cell data grouped by age. (e) Electrophoresis gel showing Telomerase expression in freshly purified SSCs from 2-mo and 24-mo mice. (f) Heatmap showing expression of tissue digest and stress associated response genes in single cell data grouped by age. (g) Heatmap showing expression of tissue digest and stress associated response genes in single cell data grouped by Leiden cluster. (h) Total read count per single cell in UMAP plot. (i) Cell cycle status of single cells illustrated in UMAP plot. (j) Proportion of cell cycle state per age group. (k) CytoTrace scores of single SSCs grouped by Leiden cluster. (l) Single cell data of selected age-associated genes related to enhanced bone loss and support of osteoclastogenesis displayed as violin plots grouped by age. (m) EnrichR gene ontology analysis of differentially expressed genes of 24-mo SSCs versus 0-mo/2-mo SSCs and their relation to cell function as determined by GO Biological Processes. Statistical testing between age groups by Student's t-test.

FIG. 10A-10G. Skeletal lineage derived Csf1 promotes bone resorption with age. (a) Model of SSC-lineage derived Csf1 actions as described in the literature for osteoclast function. (b-c) Ligand (Csf2/3) and receptor (Csf2/3r) bulk microarray gene expression (%) in the ‘2-mo’ and ‘24-mo’ SSC-lineage and the hematopoietic lineage, respectively. (d) Luminex protein data of Eotaxin1 and Tgfβ in supernatant of SSC and BCSP cultures of ‘2-mo’ and ‘24-mo’ mice (n=4). (e) Blood serum concentrations of selected inflammatory markers in ‘2-mo’ and ‘24-mo’ mouse blood (n=4-5). (f) Blood serum concentrations of Csf1, Eotaxin1, and Tgfβ in circulation of ‘2-mo’ and ‘24-mo’ mice (n=5). Statistical testing by unpaired Student's t-test with adjustments for non-normality (Mann-Whitney test) and unequal variances (Welch's correction) where appropriate. (g) Gene expression of pro-hematopoietic/pro-osteoclastic and pro-osteogenic genes in bulk RNA-sequencing data of ‘2-mo’, ‘12-mo’, and ‘24-mo’ SSCs of day-10 fracture calluses (n=3). One-tailed unpaired Student's t-test versus ‘2-mo’ with Welch's correction for unequal variances where appropriate. All data in scatter plots is shown as mean+SEM.

FIG. 11A-11G. Csf1 levels control skeletal maintenance and repair. (a) Representative μCT images of day-10 fracture calluses at time of surgery supplemented with hydrogel containing recombinant Csf1 (5 μg) or PBS as control. (b) BMD of day-10 fracture calluses treated with or without recombinant Csf1 (n=4-5). (c) Total number of SSCs and BCSPs at day-10 assessed by FACS. (d) Representative μCT reconstructions of femur bones from uninjured wild-type or haploinsufficient Csf1-KO (Csf1-KO^{+/-}) 15-mo female and male mice. (e) Trabecular BMD (top) and cortical total mineral density (TMD; bottom) of female and male WT and Csf1-KO 15-mo femur bones (n=4). (f) Bone parameters quantified by μCT from uninjured 15-mo WT and Csf1-KO female and male mice (n=4). (g) Bone parameters quantified by μCT from 21-day fracture calluses of 15-mo WT and Csf1-KO female mice (n=4). Data shown as mean+SEM. All comparison of ‘2-mo’ vs. ‘24-mo’ groups by unpaired Student's t-test adjusted for non-normality (Mann-Whitney test) and unequal variances (Welch's correction) if appropriate.

FIG. 12A-12B. Manipulation of ‘24-mo’ fracture healing by targeting the SSC lineage. (a) Frequency of BCSPs, Thy1, and 6C3+ in ‘24-mo’ mice at day-10 after fracture induction and application of factors (Bmp2: 5 μg; Csf1^{low}: 2 μg; Csf1^{high}: 5 μg) (n=5-9). (b) MicroCT analysis of newly formed mineralized bone volume of treated fracture calluses at day-21 (n=6-12). Data shown as mean+SEM. Statistical significance was calculated by Student's t-test between ‘2-mo’ and each ‘24-mo’ groups and adjusted for non-normality (Mann-Whitney test) or unequal variances (Welch's test) where appropriate.

FIG. 13A-13G. Compositional and transcriptomic changes in fracture calluses of aged mice with different treatment. (a) Leiden clustering of 10× single cell RNA-sequencing experiment of 17,230 ‘24-mo-PBS’ and ‘24-mo-aCsf1^{low}+Bmp2’ fracture callus cells. (b) UMAP plot showing expression of selected marker genes for Leiden clusters. (c) UMAP plot showing distribution of cells from each treatment group. Red: ‘24-mo-PBS’, Grey: ‘24-mo-aCsf1^{low}+Bmp2’. (d) Percentual fraction of treatment group cells per Leiden cluster. (e) Heatmap showing positive and negative markers used to identify SSCs. (f) Dotplot showing absence of lymphoid gene expression in 10× datasets. (g) UMAP plot showing expression of selected marker genes in 10× dataset subset for cells enriched for hematopoietic gene expression.

FIG. 14. Graphical abstract of SSC mediated skeletal aging. Loss of skeletal integrity with age due to reduced bone formation and increased bone resorption is associated with reduced SSC frequency and activity. The ‘24-mo’ skeleton is characterized by increased bone loss, impaired regeneration, and lineage skewing of the SSC lineage towards osteoclast-supportive stroma. Skeletal regeneration can be rejuvenated by simultaneous application of recombinant Bmp2 and a low dose of an antibody blocking Csf1 actions.

### DETAILED DESCRIPTION

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supercedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Methods, pharmaceutical compositions and kits for regenerating skeletal tissue at a targeted site in vivo are provided. In specific embodiments, compositions and methods are provided for reactivating aged mammalian skeletal stem cells through biochemical stimulation. The biochemical factors can be provided as a localized implant. In some embodiments no exogenous cells are provided, i.e. only the resident SSC are activated. Optionally the resident SSC are augmented with provision of exogenous cells, e.g. SSC or non-skeletal stem cells. In some embodiments the factors are provided in a unit dose of a drug delivery implant, which is positioned at the targeted site, e.g. to contact fully a targeted skeletal lesion.

These and other objects, advantages, and features of the invention will become apparent to those persons skilled in the art upon reading the details of the subject methods and compositions as more fully described below.

General methods in molecular and cellular biochemistry can be found in such standard textbooks as Molecular Cloning: A Laboratory Manual, 3rd Ed. (Sambrook et al., Harbor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference. Reagents, cloning vectors, and kits for genetic manipulation referred to in this disclosure are available from commercial vendors such as BioRad, Stratagene, Invitrogen, Sigma-Aldrich, and ClonTech.

The terms “polypeptide,” “peptide” and “protein” are used interchangeably herein to refer to a polymer of amino acid residues. The terms also apply to amino acid polymers in which one or more amino acid residue is an artificial chemical mimetic of a corresponding naturally occurring amino acid, as well as to naturally occurring amino acid polymers and non-naturally occurring amino acid polymer.

The term “sequence identity,” as used herein in reference to polypeptide or DNA sequences, refers to the subunit sequence identity between two molecules. When a subunit position in both of the molecules is occupied by the same monomeric subunit (e.g., the same amino acid residue or nucleotide), then the molecules are identical at that position. The similarity between two amino acid or two nucleotide sequences is a direct function of the number of identical positions. In general, the sequences are aligned so that the highest order match is obtained. If necessary, identity can be calculated using published techniques and widely available computer programs, such as the GCS program package (Devereux et al., Nucleic Acids Res. 12:387, 1984), BLASTP, BLASTN, FASTA (Atschul et al., J. Molecular Biol. 215:403, 1990).

By “protein variant” or “variant protein” or “variant polypeptide” herein is meant a protein that differs from a wild-type protein by virtue of at least one amino acid modification. The parent polypeptide may be a naturally occurring or wild-type (WT) polypeptide, or may be a modified version of a WT polypeptide. Variant polypeptide may refer to the polypeptide itself, a composition comprising the polypeptide, or the amino sequence that encodes it. Preferably, the variant polypeptide has at least one amino acid modification compared to the parent polypeptide, e.g. from about one to about ten amino acid modifications, and preferably from about one to about five amino acid modifications compared to the parent.

By “parent polypeptide”, “parent protein”, “precursor polypeptide”, or “precursor protein” as used herein is meant an unmodified polypeptide that is subsequently modified to generate a variant. A parent polypeptide may be a wild-type (or native) polypeptide, or a variant or engineered version of a wild-type polypeptide. Parent polypeptide may refer to the polypeptide itself, compositions that comprise the parent polypeptide, or the amino acid sequence that encodes it.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, gamma-carboxyglutamate, and O-phosphoserine. “Amino acid analogs” refers to compounds that have the same basic chemical structure as a naturally occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but that functions in a manner similar to a naturally occurring amino acid.

Amino acid modifications disclosed herein may include amino acid substitutions, deletions and insertions, particularly amino acid substitutions. Variant proteins may also include conservative modifications and substitutions at other positions of the cytokine and/or receptor (e.g., positions other than those involved in the affinity engineering). Such conservative substitutions include those described by Dayhoff in The Atlas of Protein Sequence and Structure 5 (1978), and by Argos in EMBO J., 8:779-785 (1989). For example, amino acids belonging to one of the following groups represent conservative changes: Group I: Ala, Pro, Gly, Gln, Asn, Ser, Thr; Group II: Cys, Ser, Tyr, Thr; Group III: Val, Ile, Leu, Met, Ala, Phe; Group IV: Lys, Arg, His; Group V: Phe, Tyr, Trp, His; and Group VI: Asp, Glu. Further, amino acid substitutions with a designated amino acid may be replaced with a conservative change.

The term “isolated” refers to a molecule that is substantially free of its natural environment. For instance, an isolated protein is substantially free of cellular material or other proteins from the cell or tissue source from which it is derived. The term refers to preparations where the isolated protein is sufficiently pure to be administered as a therapeutic composition, or at least 70% to 80% (w/w) pure, more preferably, at least 80%-90% (w/w) pure, even more preferably, 90-95% pure; and, most preferably, at least 95%, 96%, 97%, 98%, 99%, or 100% (w/w) pure. A “separated” compound refers to a compound that is removed from at least 90% of at least one component of a sample from which the compound was obtained. Any compound described herein can be provided as an isolated or separated compound.

The terms “subject,” “individual,” and “patient” are used interchangeably herein to refer to a mammal being assessed for treatment and/or being treated. In some embodiments, the mammal is a human. The terms “subject,” “individual,” and “patient” encompass, without limitation, individuals having a disease. Subjects may be human, but also include other mammals, particularly those mammals useful as laboratory models for human disease, e.g., mice, rats, etc.

The term “sample” with reference to a patient encompasses blood and other liquid samples of biological origin, solid tissue samples such as a biopsy specimen or tissue cultures or cells derived therefrom and the progeny thereof. The term also encompasses samples that have been manipulated in any way after their procurement, such as by treatment with reagents; washed; or enrichment for certain cell populations, such as diseased cells. The definition also includes samples that have been enriched for particular types of molecules, e.g., nucleic acids, polypeptides, etc. The term “biological sample” encompasses a clinical sample, and also includes tissue obtained by surgical resection, tissue obtained by biopsy, cells in culture, cell supernatants, cell lysates, tissue samples, organs, bone marrow, blood, plasma, serum, and the like. A “biological sample” includes a sample obtained from a patient's diseased cell, e.g., a sample comprising polynucleotides and/or polypeptides that is obtained from a patient's diseased cell (e.g., a cell lysate or other cell extract comprising polynucleotides and/or polypeptides); and a sample comprising diseased cells from a patient. A biological sample comprising a diseased cell from a patient can also include non-diseased cells.

The term “diagnosis” is used herein to refer to the identification of a molecular or pathological state, disease or condition in a subject, individual, or patient.

The term “prognosis” is used herein to refer to the prediction of the likelihood of death or disease progression, including recurrence, spread, and drug resistance, in a subject, individual, or patient. The term “prediction” is used herein to refer to the act of foretelling or estimating, based on observation, experience, or scientific reasoning, the likelihood of a subject, individual, or patient experiencing a particular event or clinical outcome.

As used herein, the terms “treatment,” “treating,” and the like, refer to administering an agent, or carrying out a procedure, for the purposes of obtaining an effect on or in a subject, individual, or patient. The effect may be prophylactic in terms of completely or partially preventing a disease or symptom thereof and/or may be therapeutic in terms of effecting a partial or complete cure for a disease and/or symptoms of the disease. “Treatment,” as used herein, may include treatment of a bone lesion, e.g. a fracture, in a mammal, particularly in a human, and includes: improving the regeneration of bone at a targeted site.

Treating may refer to any indicia of success in the treatment or amelioration or prevention of a condition, including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms can be based on objective or subjective parameters; including the results of an examination by a physician.

For example, age-related bone loss and regenerative decline coincide with a diminished skeletal stem cell pool with skewed lineage output. The aged skeletal phenotype is associated with distinct changes in bone architecture, including an attenuation of the growth plate, reduced bone mineral density (BMD), decreased trabecular bone mass, and a decrease in active matrix mineralization via calcein labeling, an in vivo measurement of bone formation and remodeling. Aged bones form significantly smaller calluses, and mechanical strength testing showed that these calluses were more prone to re-fracture, have less volume and were significantly less mineralized than a young skeleton. An aged skeletal phenotype is characterized by a decline in both homeostatic and regenerative bone formation.

Effectiveness in treatment can be monitored, for example, by determining post-healing bone strength by mechanical testing, determining callus size, determining matrix mineralization by calcein labeling, and the like. An effective treatment can increase one or more of these indicia by at least about 10%, at least about 20%, at least about 30%, at least about 40%, at least about 50%, or more, in an aged individual, relative to bone repair in the absence of the treatment.

As used herein, a “therapeutically effective amount” refers to that amount of the therapeutic agent sufficient to treat or manage a disease or disorder. A therapeutically effective amount may refer to the amount of therapeutic agent sufficient to improve bone regeneration as disclosed above. A therapeutically effective amount may also refer to the amount of the therapeutic agent that provides a therapeutic benefit in the treatment or management of a bone fractures and other lesions in the aged. Further, a therapeutically effective amount with respect to a therapeutic agent of the invention means the amount of therapeutic agent alone, or in combination with other therapies, that provides a therapeutic benefit in the treatment or management of bone regeneration in the aged.

As used herein, the term “dosing regimen” refers to a unit dose or doses that are administered individually to a subject, which may be separated by periods of time if multiple doses are administered. In some embodiments, a given therapeutic agent has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

“In combination with”, “combination therapy” and “combination products” refer, in certain embodiments, to the concurrent administration to a patient of the factors described herein in combination with additional therapies. When administered in combination, each component can be administered at the same time or sequentially in any order at different points in time. Thus, each component can be administered separately but sufficiently closely in time so as to provide the desired therapeutic effect.

“Concomitant administration” means administration of one or more components, such as engineered proteins and cells, known therapeutic agents, etc. at such time that the combination will have a therapeutic effect. Such concomitant administration may involve concurrent (i.e. at the same time), prior, or subsequent administration of components. A person of ordinary skill in the art would have no difficulty determining the appropriate timing, sequence and dosages of administration. In some embodiments of the present invention, active agents are co-formulated in an implant for concurrent administration.

The use of the term “in combination” does not restrict the order in which prophylactic and/or therapeutic agents are administered to a subject with a disorder. A first prophylactic or therapeutic agent can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second prophylactic or therapeutic agent to a subject with a disorder.

Aged. As used herein, the term aged refers to the effects or the characteristics of increasing age, particularly with respect to the diminished ability of somatic tissues to regenerate in response to damage, disease, and normal use. One measure of aging, therefore, is evidenced by the inability of the organism to provide suitable signals for the activation of somatic stem cells. It is shown herein that such signals are soluble factors; and thus may be empirically measured, e.g. by functional assay such as the ability of soluble factors in the patient blood to induce stem cell activation in response to tissue damage; etc.

Alternatively, aging may be defined in terms of general physiological characteristics. The rate of aging is very species specific, where a human may be aged at older than about 50 years; and a rodent at about 2 years. In general terms, a natural progressive decline in body systems starts in early adulthood, but it becomes most evident several decades later. One arbitrary way to define old age more precisely in humans is to say that it begins at conventional retirement age, older than around about 60, older than around about 65 years of age. Another definition sets parameters for aging coincident with the loss of reproductive ability, which is around about age 45, more usually around about 50 in humans, but will, however, vary with the individual. For the purposes of the present disclosure, an aged human may be greater than about 50 years of age, greater than about 55, greater than about 60, greater than about 65, greater than about 70, greater than about 75 years of age.

Antibodies. In some embodiments of the invention, a bone regenerative agent is an antibody, including, for example, an anti-CSF1 antibody. The specific or selective fit of a given structure and its specific epitope is sometimes referred to as a “lock and key” fit. The archetypal antibody molecule is the immunoglobulin, and all types of immunoglobulins, IgG, IgM, IgA, IgE, IgD, etc., from all sources, e.g. human, rodent, rabbit, cow, sheep, pig, dog, other mammal, chicken, other avians, etc., are considered to be “antibodies.” Antibodies utilized in the present invention may be polyclonal antibodies, although monoclonal antibodies are preferred because they may be reproduced by cell culture or recombinantly, and can be modified to reduce their antigenicity.

Polyclonal antibodies can be raised by a standard protocol by injecting a production animal with an antigenic composition, formulated as described above. See, e.g., Harlow and Lane, Antibodies: A Laboratory Manual, Cold Spring Harbor Laboratory, 1988. In one such technique, an antigen comprising an antigenic portion of the target polypeptide is initially injected into any of a wide variety of mammals (e.g., mice, rats, rabbits, sheep or goats). When utilizing an entire protein, or a larger section of the protein, antibodies may be raised by immunizing the production animal with the protein and a suitable adjuvant (e.g., Fruend's, Fruend's complete, oil-in-water emulsions, etc.) When a smaller peptide is utilized, it is advantageous to conjugate the peptide with a larger molecule to make an immunostimulatory conjugate. Commonly utilized conjugate proteins that are commercially available for such use include bovine serum albumin (BSA) and keyhole limpet hemocyanin (KLH). In order to raise antibodies to particular epitopes, peptides derived from the full sequence may be utilized. Alternatively, in order to generate antibodies to relatively short peptide portions of the brain tumor protein target, a superior immune response may be elicited if the polypeptide is joined to a carrier protein, such as ovalbumin, BSA or KLH. The peptide-conjugate is injected into the animal host, preferably according to a predetermined schedule incorporating one or more booster immunizations, and the animals are bled periodically. Polyclonal antibodies specific for the polypeptide may then be purified from such antisera by, for example, affinity chromatography using the polypeptide coupled to a suitable solid support.

Alternatively, for monoclonal antibodies, hybridomas may be formed by isolating the stimulated immune cells, such as those from the spleen of the inoculated animal. These cells are then fused to immortalized cells, such as myeloma cells or transformed cells, which are capable of replicating indefinitely in cell culture, thereby producing an immortal, immunoglobulin-secreting cell line.

In addition, the antibodies or antigen binding fragments may be produced by genetic engineering. In this technique, as with the standard hybridoma procedure, antibody-producing cells are sensitized to the desired antigen or immunogen. The messenger RNA isolated from the immune spleen cells or hybridomas is used as a template to make cDNA using PCR amplification. A library of vectors, each containing one heavy chain gene and one light chain gene retaining the initial antigen specificity, is produced by insertion of appropriate sections of the amplified immunoglobulin cDNA into the expression vectors. A combinatorial library is constructed by combining the heavy chain gene library with the light chain gene library. This results in a library of clones which co-express a heavy and light chain (resembling the Fab fragment or antigen binding fragment of an antibody molecule). The vectors that carry these genes are co-transfected into a host (e.g. bacteria, insect cells, mammalian cells, or other suitable protein production host cell.). When antibody gene synthesis is induced in the transfected host, the heavy and light chain proteins self-assemble to produce active antibodies that can be detected by screening with the antigen or immunogen.

Antibodies with a reduced propensity to induce a violent or detrimental immune response in humans (such as anaphylactic shock), and which also exhibit a reduced propensity for priming an immune response which would prevent repeated dosage with the antibody therapeutic or imaging agent are preferred for use in the invention. Thus, humanized, chimeric, or xenogenic human antibodies, which produce less of an immune response when administered to humans, are preferred for use in the present invention.

In addition to full-length immunoglobulins that comprise hinge and Fc region sequences (or their recombinant counterparts), immunoglobulin fragments comprising the epitope binding site (e.g., Fab′, F(ab′)₂, or other fragments) are useful as antibody moieties in the present invention. Such antibody fragments may be generated from whole immunoglobulins by trypsin, pepsin, papain, or other protease cleavage. “Fragment,” or minimal immunoglobulins may be designed utilizing recombinant immunoglobulin techniques. For instance “Fv” immunoglobulins for use in the present invention may be produced by linking a variable light chain region to a variable heavy chain reg′ion via a peptide linker (e.g., poly-glycine or another sequence which does not form an alpha helix or beta sheet motif). Fv fragments are heterodimers of the variable heavy chain domain (VH) and the variable light chain domain (VL). The heterodimers of heavy and light chain domains that occur in whole IgG, for example, are connected by a disulfide bond. Recombinant Fvs in which VH and VL are connected by a peptide linker are typically stable, see, for example, Huston et al., Proc. Natl. Acad, Sci. USA 85:5879-5883 (1988) and Bird et al., Science 242:423-426 (1988), both fully incorporated herein, by reference. These are single chain Fvs which have been found to retain specificity and affinity and have been shown to be useful for imaging tumors and to make recombinant immunotoxins for tumor therapy. However, researchers have bound that some of the single chain Fvs have a reduced affinity for antigen and the peptide linker can interfere with binding. Improved Fv's have been also been made which comprise stabilizing disulfide bonds between the V.sub.H and V.sub.L regions, as described in U.S. Pat. No. 6,147,203, incorporated fully herein by reference. Any of these minimal antibodies may be utilized in the present invention, and those which are humanized to avoid HAMA reactions are preferred for use in embodiments of the invention.

Candidate antibodies can be tested for activity by a variety of methods. As a first screen, the antibodies may be tested for binding against a CSF1 protein of interest. After selective binding to the target is established, the candidate antibody may be tested for appropriate activity in an in vivo model, such as an appropriate cell line, or in an animal model. Antibodies may be assayed in functional formats, such as inducing stem cells to enter cell cycle; proliferation of stem cells, production of differentiated cells from stem cells; and the like, which may be assessed in culture or in an animal system.

CSF1. The colony stimulating factor 1 (CSF1), also known as macrophage colony-stimulating factor (M-CSF), is a secreted cytokine. The active form of the protein is found extracellularly as a disulfide-linked homodimer, and is thought to be produced by proteolytic cleavage of membrane-bound precursors. Four transcript variants encoding three different isoforms have been found for this gene. The role of M-CSF is not only restricted to the monocyte/macrophage cell lineage. By interacting with its membrane receptor (CSF1R or M-CSF-R encoded by the c-fms proto-oncogene), M-CSF also modulates the proliferation of earlier hematopoietic progenitors and influence numerous physiological processes involved in immunology, metabolism, fertility and pregnancy. M-CSF released by osteoblasts exerts paracrine effects on osteoclasts.

Inhibitors of human CSF1 are known and used in the art. CSF1 inhibitors can be, to name just a few examples, small molecules, peptides, polypeptides, proteins, including more specifically antibodies, including anti-CSF1 antibodies, anti-CSF1R antibodies, intrabodies, maxibodies, minibodies, diabodies, Fc fusion proteins such as peptibodies, receptibodies, soluble CSF1 receptor proteins and fragments, and a variety of others. CSF1 inhibitors in accordance with the invention include small molecules and antibodies that target the receptor, CSF1R, or the ligand, CSF1. Antibodies and small molecules for this purpose that are currently enrolled in clinical trials include, for example:

| | | | ClinicalTrials.gov |
|---|---|---|---|
| Class | Target | Compound | identifier |
| Small | CSF1R | Pexidartinib | NCT02071940 |
| molecules | | (PLX3397, PLX108-01) | |
| | CSF1R | PLX7486 | NCT01804530 |
| | CSF1R | ARRY-382 | NCT01316822 |
| | CSF1R | JNJ-40346527 | NCT01572519 |
| | CSF1R | BLZ945 | NCT02829723 |
| Monoclonal | CSF1R | Emactuzumab | NCT01494688 |
| antibodies | | (RG7155) | |
| | CSF1R | AMG820 | NCT01444404 |
| | CSF1R | IMC-CS4 (LY3022855) | NCT01346358 |
| | CSF1 | MCS110 | NCT00757757 |

Other anti-human CSF1 antibodies include, without limitation, Human Anti-CSF1 Recombinant Antibody (clone 100); scFv Fragment (CAT #: HPAB-0749-WJ-S(P), Creative Biolabs); Antagonistic CSF-1R Monoclonal Antibody Cabiralizumab (BMS-986227); and anti-CSF1 Monoclonal Antibody PD-0360324. Alternatively, suitable antibodies can be generated for this purpose.

The dose of anti-CSF1 provided in a unit dose in an composition, e.g. an implant, for a mouse model will depend on the specific inhibitor but for an antibody may be, for example, from about 0.5 μg; about 1 μg; about 1.5 μg; about 2 μg; about 2.5 μg; and not more than about 5 μg; not more than about 4 μg; not more than about 2.5 μg. The corresponding unit dose for a human, e.g. an aged human, will be corresponding higher, and may be at least about 20 μg; at least about 25 μg; at least about 50 μg; at least about 75 μg; at least about 100 μg; and up to about 10 mg, up to about 5 mg, up to about 1 mg, up to about 500 μg, up to about 250 μg, up to about 150 μg. The dose of other anti-CSF1 agents, e.g. small molecule and antibody inhibitors of CSF1R, may be provided in a unit dose that is appropriately scaled to be comparable to these levels of anti-CSF1 antibody.

As used herein, the term “BMP-2” refers to the family of bone morphogenetic proteins of the type 2, derived from any species, and may include mimetics and variants thereof. Reference to BMP2 herein is understood to be a reference to any one of the currently identified forms, including BMP2A and BMP2B, as well as to BMP2 species identified in the future. The term “BMP2” also includes polypeptides derived from the sequence of any known BMP2 whose mature sequence is at least about 75% homologous with the sequence of a mature human BMP2, which reference sequence may be found in Genbank, accession number NP_001191.

BMP2 signals via two types of receptors (BRI and BRII) that are expressed at the cell surface as homomeric as well as heteromeric complexes. Prior to ligand binding, a low but measurable level of BMP-receptors is found in preformed hetero-oligomeric complexes. The major fraction of the receptors is recruited into hetero-oligomeric complexes only after ligand addition. For this, BMP2 binds first to the high affinity receptor BRI and then recruits BRII into the signaling complex. However, ligand binding to the preformed complex composed of BRII and BRI is still required for signaling, suggesting that it may mediate activating conformational changes. Signals induced by binding of BMP2 to preformed receptor complexes activate the Smad pathway, whereas BMP2-induced recruitment of receptors activates a different, Smad-independent pathway resulting in the induction of alkaline phosphatase activity via p38 MAPK.

“BMP2 agents” include molecules that function similarly to BMP2 by binding and activating its receptors as described above. Molecules useful as BMP2 agents include derivatives, variants, and biologically active fragments of naturally occurring BMP2. A “variant” polypeptide means a biologically active polypeptide as defined below having less than 100% sequence identity with a native sequence polypeptide. Such variants include polypeptides wherein one or more amino acid residues are added at the N- or C-terminus of, or within, the native sequence; from about one to forty amino acid residues are deleted, and optionally substituted by one or more amino acid residues; and derivatives of the above polypeptides, wherein an amino acid residue has been covalently modified so that the resulting product has a non-naturally occurring amino acid. Ordinarily, a biologically active variant will have an amino acid sequence having at least about 90% amino acid sequence identity with a native sequence polypeptide, preferably at least about 95%, more preferably at least about 99%. The variant polypeptides can be naturally or non-naturally glycosylated, i.e., the polypeptide has a glycosylation pattern that differs from the glycosylation pattern found in the corresponding naturally occurring protein. The variant polypeptides can have post-translational modifications not found on the natural BMP2 protein.

Fragments and fusion proteins of soluble BMP2, particularly biologically active fragments and/or fragments corresponding to functional domains, are of interest. Fragments of interest will typically be at least about 10 aa to at least about 15 aa in length, usually at least about 50 aa in length, but will usually not exceed about 142 aa in length, where the fragment will have a stretch of amino acids that is identical to BMP2. A fragment “at least 20 aa in length,” for example, is intended to include 20 or more contiguous amino acids from, for example, the polypeptide encoded by a cDNA for BMP2. In this context “about” includes the particularly recited value or a value larger or smaller by several (5, 4, 3, 2, or 1) amino acids. The protein variants described herein are encoded by polynucleotides that are within the scope of the invention. The genetic code can be used to select the appropriate codons to construct the corresponding variants. The polynucleotides may be used to produce polypeptides, and these polypeptides may be used to produce antibodies by known methods.

In some embodiments, a dose of BMP2 is provided in an implant, e.g. a matrix or scaffold for localized delivery of the factor, where the BMP2 is provided as a BMP2 protein or active fragment thereof. The effective dose may be determined based on the specific tissue, rate of release from the implant, size of the implant, and the like. and may be empirically determined by one of skill in the art. The dose may provide for biological activity equivalent to 1 μg BMP2 protein, 10 μg, 100 μg, 1 mg, 5 mg, 10 mg, 25 mg, 50 mg, 75 mg, 100 mg, 250 mg, 500 mg, 750 mg, 1 g of BMP2 protein. The dose may be administered at a single time point, e.g. as a single implant; or may be fractionated, e.g. delivered in a microneedle configuration. The dose may be administered, once, two, three time, 4 times, 5 times, 10 times, or mare as required to achieve the desired effect, and administration may be daily, every 2 days, every 3 days, every 4 days, weekly, bi-weekly, monthly, or more.

VEGF is a dimeric, disulfide-linked 46-kDa glycoprotein related to Platelet-Derived Growth Factor (“PDGF”). It is produced by normal cell lines and tumor cell lines; is an endothelial cell-selective mitogen; shows angiogenic activity in in vivo test systems (e.g., rabbit cornea); is chemotactic for endothelial cells and monocytes; and induces plasminogen activators in endothelial cells, which are involved in the proteolytic degradation of the extracellular matrix during the formation of capillaries.

“VEGF inhibitor” as used herein is any substance that decreases signaling by the VEGF-VEGFR pathway. VEGF inhibitors can be, to name just a few examples, small molecules, peptides, polypeptides, proteins, including more specifically antibodies, including anti-VEGF antibodies, anti-VEGFR antibodies, intrabodies, maxibodies, minibodies, diabodies, Fc fusion proteins such as peptibodies, receptibodies, soluble VEGF receptor proteins and fragments, and a variety of others. Many VEGF inhibitors work by binding to VEGF or to a VEGF receptor. Others work more indirectly by binding to factors that bind to VEGF or to a VEGF receptor or to other components of the VEGF signaling pathway. Still other VEGF inhibitors act by altering regulatory posttranslational modifications that modulate VEGF pathway signaling. VEGF inhibitors in accordance with the invention also may act through more indirect mechanisms. Whatever the mechanism involved, as used herein, a VEGF inhibitor decreases the effective activity of the VEGF signaling pathway in a given circumstance over what it would be in the same circumstance in the absence of the inhibitor.

In some embodiments, a dose of VEGF inhibitor is provided in an implant, e.g. a matrix or scaffold for localized delivery of the factor. The effective dose may be determined based on the specific tissue, rate of release from the implant, size of the implant, and the like. and may be empirically determined by one of skill in the art. The dose may provide for biological activity equivalent to 1 μg soluble VEGF receptor, 10 μg, 100 μg, 1 mg, 5 mg, 10 mg, 25 mg, 50 mg, 75 mg, 100 mg, 250 mg, 500 mg, 750 mg, 1 g of soluble VEGF receptor. The dose may be administered at a single time point, e.g. as a single implant; or may be fractionated, e.g. delivered in a microneedle configuration. The dose may be administered, once, two, three time, 4 times, 5 times, 10 times, or mare as required to achieve the desired effect, and administration may be daily, every 2 days, every 3 days, every 4 days, weekly, bi-weekly, monthly, or more.

A great many VEGF inhibitors have been described in the literature. In addition to those described in further detail below, VEGF inhibitors are described in the following patent documents: US 2003/0105091, US2006/0241115, U.S. Pat. Nos. 5,521,184, 5,770,599, 5,990,141, 6,235,764, 6,258,812, 6,515,004, 6,630,500, 6,713,485, WO2005/070891, WO 01/32651, WO 02/68406, WO 02/66470, WO 02/55501, WO 04/05279, WO 04/07481, WO 04/07458, WO 04/09784, WO 02/59110, WO 99/450029, WO 00/59509, WO 99/61422, WO 00/12089, WO 00/02871, and WO 01/37820, particularly in parts pertinent to VEGF inhibitors.

The following are among specific VEGF inhibitors: ABT-869 (Abbott) including formulations for oral administration and closely related VEGF inhibitors; AEE-788 (Novartis) (also called AE-788 and NVP-AEE-788, among others) including formulations for oral administration and closely related VEGF inhibitors; AG-13736 (Pfizer) (also called AG-013736) including formulations for oral administration and closely related VEGF inhibitors; AG-028262 (Pfizer) and closely related VEGF inhibitors; Angiostatin (EntreMed) (also called CAS Registry Number 86090-08-6, K1-4, and rhuAngiostatin, among others) and closely related inhibitors as described in, among others, U.S. Pat. Nos. 5,792,825 and 6,025,688, particularly in parts pertaining to Angiostatin and closely related VEGF inhibitors, their structures and properties, and methods for making and using them; Avastin™ (Genentech) (also called bevacizumab, R-435, rhuMAB-VEGF, and CAS Registry Number 216974-75-3, among others) and closely related VEGF inhibitors; AVE-8062 (Ajinomoto Co. and Sanofi-aventis) (also called AC-7700 and combretastatin A4 analog, among others), and closely related VEGF inhibitors; AZD-2171 (AstraZeneca) and closely related VEGF inhibitors; Nexavar® (Bayer AG and Onyx) (also called CAS Registry Number 284461-73-0, BAY-43-9006, raf kinase inhibitor, sorafenib, sorafenib analogs, and IDDBCP150446, among others) and closely related VEGF inhibitors; BMS-387032 (Sunesis and Bristol-Myers Squibb) (also called SNS-032 and CAS Registry Number 345627-80-7, among others) and closely related VEGF inhibitors; CEP-7055 (Cephalon and Sanofi-aventis) (also called CEP-11981 and SSR-106462, among others) and closely related VEGF inhibitors; CHIR-258 (Chiron) (also called CAS Registry Number 405169-16-6, GFKI, and GFKI-258, among others) and closely related VEGF inhibitors; CP-547632 (OSI Pharmaceuticals and Pfizer) (also called CAS Registry Number 252003-65-9, among others) and closely related VEGF inhibitors such as, for instance, CP-564959; E-7080 (Eisai Co.) (also called CAS Registry Number 417716-92-8 and ER-203492-00, among others) and closely related VEGF inhibitors; 786034 (GlaxoSmithKline) and closely related VEGF inhibitors; GW-654652 (GlaxoSmithKline) and closely related indazolylpyrimidine Kdr inhibitors; IMC-1C11 (ImClone) (also called DC-101 and c-p1C11, among others) and closely related VEGF inhibitors; KRN-951 (Kirin Brewery Co.) and other closely related quinoline-urea VEGF inhibitors; PKC-412 (Novartis) (also called CAS Registry Number 120685-11-2, benzoylstaurosporine, CGP-41251, midostaurin, and STI-412, among others) and closely related VEGF inhibitors; PTK-787 (Novartis and Schering) (also called CAS Registry Numbers 212141-54-3 and 212142-18-2, PTK/ZK, PTK-787/ZK-222584, ZK-22584, VEGF-TKI, VEGF-RKI, PTK-787A, DE-00268, CGP-79787, CGP-79787D, vatalanib, ZK-222584, among others) and closely related anilinophthalazine derivative VEGF inhibitors; SU11248 (Sugen and Pfizer) (also called SU-11248, SU-011248, SU-11248J, Sutent®, and sunitinib malate, among others) and closely related VEGF inhibitors; SU-5416 (Sugen and Pfizer/Pharmacia) (also called CAS Registry Number 194413-58-6, semaxanib, 204005-46-9, among others) and closely related VEGF inhibitors; SU-6668 (Sugen and Taiho) (also called CAS Registry Number 252916-29-3, SU-006668, and TSU-68, among others) and closely related VEGF inhibitors as described in, among others, WO-09948868, WO-09961422, and WO-00038519, particularly in parts pertaining to SU-6668 and closely related VEGF inhibitors, their structures and properties, and methods for making and using them; VEGF Trap (Regeneron and Sanofi-aventis) (also called AVE-0005 and Systemic VEGF Trap, among others) and closely related VEGF inhibitors as described in, among others, WO-2004110490, particularly in parts pertaining to VEGF Trap and closely related VEGF inhibitors, their structures and properties, and methods for making and using them; Thalidomide (Celgene) (also called CAS Registry Number 50-35-1, Synovir, Thalidomide Pharmion, and Thalomid, among others) and closely related VEGF inhibitors; XL-647 (Exelixis) (also called EXEL-7647, among others) and closely related VEGF inhibitors; XL-999 (Exelixis) (also called EXEL-0999, among others) and closely related VEGF inhibitors; XL-880 (Exelixis) (also called EXEL-2880, among others) and closely related VEGF inhibitors; ZD-6474 (AstraZeneca) (also called CAS Registry Number 443913-73-3, Zactima, and AZD-6474, among others) and closely related anilinoquinazoline VEGF inhibitors; and ZK-304709 (Schering) (also called CDK inhibitors (indirubin derivatives), ZK-CDK, MTGI, and multi-target tumor growth inhibitor, among others) and other closely related compounds including the indirubin derivative VEGF inhibitors described in WO-00234717, WO-02074742, WO-02100401, WO-00244148, WO-02096888, WO-03029223, WO-02092079, and WO-02094814, particularly in parts pertinent to these and closely related VEGF inhibitors, their structures and properties, and methods for making and using them.

VEGF inhibitors may be delivered in a manner appropriate to the nature of the inhibitor, e.g. as a protein, small molecule, nucleic acid, etc., including without limitation appropriate vehicles and vectors as required.

Hedgehog agent. As used herein, the term “hedgehog agent” or “agent that provides for hedgehog activity” refers to any agent that provides for the same activity in the signaling pathway as a native hedgehog protein on its homologous, cognate receptor, for example an agent may have at least about 20% of the native protein activity, at least about 30%, at least about 40%, at least about 50%, at least about 60%, at least about 70%, at least about 80%, at least about 90%, at least about 100%, or may have greater activity than the native protein, e.g. 2-fold, 3-fold, 5-fold, 10-fold or more activity. Levels of activity may be determined, for example, by assessing transcription of Ci target genes, processing of Ci, etc. Hedgehog (Hh) proteins are secreted morphogens that are essential for multiple developmental processes in both invertebrates and vertebrates. Secreted active Hh fragments can regulate cellular activities of neighboring and distant cells.

Hh-target cells express two components of the Hh signaling system on the cell surface: Patched (Ptc), a 12-transmembrane protein, and Smoothened (Smo), a 7-transmembrane protein. In the absence of Hh, Ptc represses the activity of Smo, which allows proteolytic processing of a downstream zinc-finger transcription factor, Cubitus intereptus (Ci) at its C-terminal end forming a transcriptional repressor. When Hh binds to Ptc it relieves Ptc repression of Smo and activated Smo stabilizes intact Ci, which then acts as a transcription activator, and hence stimulates transcription of target genes. In mammals there are two Ptc homologues, where both bind Hh proteins with similar affinity and both can interact with mammalian Smo. Ptc1 is widely expressed throughout the mouse embryo and serves as the extracellular receptor for multiple Hh proteins, and is itself upregulated by Hh signaling. Ptc2 is expressed at high levels in the skin and spermatocytes.

There are three vertebrate Hh proteins: Desert hedgehog (Dhh), Sonic hedgehog (Shh), and Indian hedgehog (Ihh). All of them have unique sets of functions in regulation of different developmental processes. Dhh is essential for the development of peripheral nerves and spermatogenesis. Shh is involved in establishing lateral asymmetry, the anterior-posterior limb axis, and development of the central nervous system. Ihh is a master regulator of endochondral bone development.

The hedgehog protein is initially synthesized as a 46 kDa precursor, with two distinct domains: the N-terminal “hedge” domain is processed to a 19 kDa fragment (Hh-N) following proteolytic cleavage that is executed by the C-terminal “hog” domain within the endoplasmic reticulum. The C-terminus acts as a cholesterol transferase to covalently attach a cholesterol group to the carboxy end of the Hh amino terminal fragment, Hh-N. The nascent Hh-N is further modified by the subsequent addition of a palmitoyl group at Cys-24, resulting in an extremely hydrophobic molecule that is referred to as Hh-Np for Hh-N-processed. The processing of Hh-N takes place in the secretory pathway and is mediated by a palmitoylacyltransferase which is coded for by the Skinny hedgehog gene (Ski/Skn). The palmitoyl addition is essential for SHH function. The addition of cholesterol and palmitate increases the efficacy of SHH-Np, while addition of hydrophilic adducts to the N terminus reduces the activity of SHH.

Protein sequences of exemplary hedgehog proteins, e.g. human hedgehog proteins, are publicly available at Genbank. Included are sonic hedgehog protein isoform 1, accession NP_000184.1; sonic hedgehog protein isoform 2, accession NP_001297391.1; indian hedgehog protein, accession number NP_002172; and desert hedgehog protein, accession NP_066382, the sequences thus identified are each specifically incorporated by reference.

Antibodies that specifically bind to human patched or smoothened are known in the art or can be generated by conventional methods. Such antibodies may be screened for agonist activity for use in the methods of the invention. Alternatively, small molecule agonists are known in the art, see, for example Frank-Kamenetsky et al. (2002) J. Biol. 1(2):10, herein specifically incorporated by reference. Specific agonists of interest include, without limitation N-Methyl-N′-(3-pyridinylbenzyl)-N′-(3-chlorobenzo[b]thiophene-2-carbonyl)-1,4-diaminocyclohexane, SAG1.1, SAG1.3, purmorphamine, etc., as described in Das et al. (2013) Sci Transl Med. 5(201):201ra120; Carney and Ingham BMC Biology 201311:37, etc.

The term “skeletal stem cell” refers to a multipotent and self-renewing cell capable of generating bone marrow stromal cells, skeletal cells, and chondrogenic cells. By self-renewing, it is meant that when they undergo mitosis, they produce at least one daughter cell that is a skeletal stem cell. By multipotent it is meant that it is capable of giving rise to progenitor cell (skeletal progenitors) that give rise to all cell types of the skeletal system. They are not pluripotent, that is, they are not capable of giving rise to cells of other organs in vivo.

Skeletal stem cells can be reprogrammed from non-skeletal cells, including without limitation mesenchymal stem cells, and adipose tissue containing such cells, such as human adipose stem cells (hAASC). Induced skeletal cells have characteristics of functional SSCs derived from nature, that is, they can give rise to the same lineages. Human SSC have a phenotype as disclosed in U.S. Pat. No. 11,083,755, herein specifically incorporated by reference.

Human SSC cell populations may be characterized by their cell surface markers, although it will be understood by one of skill in the art that endogenous populations of SSC need not be characterized for effective stimulation. Human SSC are negative for expression of CD45, CD235, Tie2, and CD31; and positively express podoplanin (PDPN). A population of cells, e.g. cells isolated from bone tissue, having this combination of markers may be referred to as [PDPN⁺/146⁻] cells. The [PDPN⁺/146⁻] population can be further subdivided into three populations: a unipotent subset capable of chondrogenesis [PDPN⁺CD146⁻CD73⁻CD164⁻], a unipotent cellular subpopulation capable of osteogenesis [PDPN⁺CD146⁻CD73⁻CD164⁺] and a multipotent [PDPN⁺CD146⁻CD73⁺CD164⁺] cell capable of endochondral (bone and cartilage) ossification. A population of cells of interest for use in the methods of the invention may be isolated from bone with respect to CD45, CD235, Tie2, and CD31 and PDPN. Other cell populations of interest are [PDPN⁺CD146⁻CD73⁻CD164⁻] cells; [PDPN⁺CD146⁻CD73⁻CD164⁺] cells; and [PDPN⁺CD146⁻CD73⁺CD164⁺] cells.

The mouse skeletal lineage is characterized as CD45−, Ter119−, Tie2−, αv integrin+. The SSC is further characterized as Thy1−6C3−CD105−CD200+.

Adipose-Derived Stem Cells. Adipose-derived stem cells or “adipose-derived stromal cells” refer to cells that originate from adipose tissue. By “adipose” is meant any fat tissue. The adipose tissue may be brown or white adipose tissue, derived from subcutaneous, omental/visceral, mammary, gonadal, or other adipose tissue site. Preferably, the adipose is subcutaneous white adipose tissue. Such cells may be provided as a primary cell culture or an immortalized cell line. The adipose tissue may be from any organism having fat tissue. Preferably, the adipose tissue is mammalian, most preferably the adipose tissue is human. A convenient source of adipose tissue is from liposuction surgery, however, the source of adipose tissue or the method of isolation of adipose tissue is not critical to the invention.

Adipose tissue is abundant and accessible to harvest methods with minimal risk to the patient. It is estimated that there are more than 10⁴ stem cells per gram of adipose tissue (Sen et al 2001, Journal of Cellular Biochemistry 81:312-319), which cells can be used immediately or cryopreserved for future autologous or allogeneic applications.

Methods for the isolation, expansion, and differentiation of human adipose tissue-derived cells have been reported. See for example, Burris et al 1999, Mol Endocrinol 13:410-7; Erickson et al 2002, Biochem Biophys Res Commun. Jan. 18, 2002; 290(2):763-9; Gronthos et al 2001, Journal of Cellular Physiology, 189:54-63; Halvorsen et al 2001, Metabolism 50:407-413; Halvorsen et al 2001, Tissue Eng. 7(6):729-41; Harp et al 2001, Biochem Biophys Res Commun 281:907-912; Saladin et al 1999, Cell Growth & Diff 10:43-48; Sen et al 2001, Journal of Cellular Biochemistry 81:312-319; Zhou et al 1999, Biotechnol. Techniques 13: 513-517. Adipose tissue-derived stromal cells may be obtained from minced human adipose tissue by collagenase digestion and differential centrifugation [Halvorsen et al 2001, Metabolism 50:407-413; Hauner et al 1989, J Clin Invest 84:1663-1670; Rodbell et al 1966, J Biol Chem 241:130-139].

Adipose tissue derived stem cells have been reported to express markers including: CD13, CD29, CD44, CD63, CD73, CD90, CD166, aldehyde dehydrogenase (ALDH), and ABCG2. The adipose tissue derived stem cells may be a population of purified mononuclear cells extracted from adipose tissue capable of proliferating in culture for more than 1 month.

For isolation of cells from tissue, an appropriate solution may be used for dispersion or suspension. Such solution will generally be a balanced salt solution, e.g. normal saline, PBS, Hank's balanced salt solution, etc., conveniently supplemented with fetal calf serum or other naturally occurring factors, in conjunction with an acceptable buffer at low concentration, generally from 5-25 mM. Convenient buffers include HEPES, phosphate buffers, lactate buffers, etc.

The cell population may be used immediately. Alternatively, the cell population may be frozen at liquid nitrogen temperatures and stored for long periods of time, being thawed and capable of being reused. In such cases, the cells will usually be frozen in 10% DMSO, 50% serum, 40% buffered medium, or some other such solution as is commonly used in the art to preserve cells at such freezing temperatures, and thawed in a manner as commonly known in the art for thawing frozen cultured cells.

The adipose cells may be cultured in vitro under various culture conditions. Culture medium may be liquid or semi-solid, e.g. containing agar, methylcellulose, etc. The cell population may be conveniently suspended in an appropriate nutrient medium, such as Iscove's modified DMEM or RPMI-1640, normally supplemented with fetal calf serum (about 5-10%), L-glutamine, a thiol, particularly 2-mercaptoethanol, and antibiotics, e.g. penicillin and streptomycin. In one embodiment of the invention, the adipose cells are maintained in culture in the absence of feeder layer cells, i.e. in the absence of serum, etc. The culture may contain growth factors to which the cells are responsive. Growth factors, as defined herein, are molecules capable of promoting survival, growth and/or differentiation of cells, either in culture or in the intact tissue, through specific effects on a transmembrane receptor. Growth factors include polypeptides and non-polypeptide factors.

The terms “efficiency of reactivation”, “reactivation efficiency” are used interchangeably herein to refer to the ability of cells to become responsive to growth and differentiation factors, for example, the ability of adipose tissue cells to give rise to iSSC when contacted with high doses of BMP2. In other words, the cells produce about 1.5-fold, about 2-fold, about 3-fold, about 4-fold, about 6-fold, about 8-fold, about 10-fold, about 20-fold, about 30-fold, about 50-fold, about 100-fold, about 200-fold the number of induced cells (e.g. iSSC) as the uncontacted population, or more.

Mammalian species that may be treated with the present methods include canines and felines; equines; bovines; ovines; etc. and primates, particularly humans. Animal models, particularly small mammals, e.g. murine, lagomorpha, etc. may be used for experimental investigations.

More particularly, the present invention finds use in the treatment of subjects, such as human patients, in need of bone regenerative therapy. Examples of such subjects would be subjects suffering from bone fractures and other lesions, particularly aged individuals. Other conditions include osteoarthritis, genetic defects, disease, etc. Patients having diseases and disorders characterized by such conditions will benefit greatly by a treatment protocol of the pending claimed invention.

An effective amount of a pharmaceutical composition of the invention is the amount that will result in an increase the activation of resident SSC at the site of implant; that will result in greater mineralization and mechanical strength in healed bones, a larger bone callus on healing, and the like. For example, an effective amount of a pharmaceutical composition will increase bone mass or mineralization at a lesion by at least about 5%, at least about 10%, at least about 20%, preferably from about 20% to about 50%, and even more preferably, by greater than 50% (e.g., from about 50% to about 100%) as compared to the appropriate control, the control typically being a subject not treated with the composition.

The methods of the present invention also find use in combined therapies, e.g. in with therapies that are already known in the art to provide relief from symptoms associated with the aforementioned diseases, disorders and conditions. For example, therapies drawn to increasing bone density include administration of antiresorptive drugs and anabolic drugs, for example alendronate, risedronate, ibandronate, zoledronic acid, etc. as known in the art. The combined use of a pharmaceutical composition of the present invention and these other agents may have the advantages that the required dosages for the individual drugs is lower, and the effect of the different drugs complementary.

In some embodiments an effective dose of mesenchymal stem cells, such as adipose stromal cells, preferably adipose derived stem cells, are optionally provided in an implant or scaffold for regeneration of tissue. An effective cell dose may depend on the purity of the population. In some embodiments an effective dose delivers a dose of adipose derived stem cells of at least about 10², about 10³, about 10⁴, about 10⁵, about 10⁶, about 10⁷, about 10⁸, about 10⁹ or more cells, which stem cells may be present in the cell population at a concentration of about 1%, about 5%, about 10%, about 20%, about 30%, about 40%, about 50%, about 60%, about 70%, about 80%, about 90% or more.

Drug delivery devices include structures that can be implanted and that release the active agents, e.g. BMP2 and CSF1 inhibitor, at the targeted site. Implantable drug delivery devices can be broadly classified in two main groups: passive implants and active implants. The first group includes two main types of implants: biodegradable and non-biodegradable implants. Active systems rely on energy dependent methods that provide the driving force to control drug release. The second group includes devices such as osmotic pressure gradients and electromechanical drives.

Passive polymeric Implants are normally relatively simple devices with no moving parts, they rely on passive diffusion for drug release. They are generally made of drugs packed within a biocompatible polymer molecule. Several parameters such as: drug type/concentration, polymer type, implant design and surface properties can be modified to control the release profile. Passive implants can be classified in two main categories: non-biodegradable and biodegradable systems.

Non-biodegradable implants are commonly prepared using polymers such as silicones, poly(urethanes), poly(acrylates) or copolymers such as poly(ethyelene vinyl acetate). Poly(ethylene-vinyl acetate) (PEVA) is a thermoplastic copolymer of ethylene and vinyl acetate. Poly(siloxanes) or silicones are organosilicon polymeric materials composed of silicon and oxygen atoms. Lateral groups can be methyl, vinyl or phenyl groups. These groups will influence the properties of the polymer. Poly(siloxanes) have been extensively used in medicine due to the unique combination of thermal stability, biocompatibility, chemical inertness and elastomeric properties. The silicones commonly used for medical devices are vulcanised at room temperature. They are prepared using a two-component poly(dimethylsiloxanes) (PDMS) in the presence of a catalyst (platinum based compound). The final material is formed via an addition hydrosilation reaction. An alternative method to obtain silicones for medical applications is the using linear PDMS with hydroxyl terminal groups. This linear polymer is cross-linked with low molecular weight tetra(alkyloxysilane) using stannous octoate catalyst.

This type of device can be monolithic or reservoir type implant. Monolithic type implants are made from a polymer matrix in which the drug is homogeneously dispersed. On the other hand, reservoir-type implants contain a compact drug core covered by a permeable non-biodegradable membrane. The membrane thickness and the permeability of the drug through the membrane will govern the release kinetics.

Biodegradable implants are made using polymers or block copolymers that can be broken down into smaller fragments that will be subsequently excreted or absorbed by the body. Normally they are made using polymers such as collagen, PEG, chitin, poly(caprolactone) (PCL), poly(lactic acid) (PLA) or poly(lactic-co-glycolic acid) (PLGA). Numerous other biodegradable polymers for drug delivery exist including: poly(amides), poly(anhydrides), poly(phosphazenes) and poly(dioxanone). Poly(anhydrides) have a low hydrolytic stability resulting in rapid degradation rates, making them suitable for use in short-term controlled delivery systems. Poly(phosphazenes) have a degradation rate that can be finely tuned by appropriate substitution with specific chemical groups and use of these polymers has been investigated for skeletal tissue regeneration and drug delivery. Poly(dioxanone), like PCL, is a polylactone that has been used for purposes such as drug delivery, and tissue engineering They do not need to be extracted after implantation, as they will be degraded by the body of the patient. They can be manufactured as monolithic implants and reservoir-type implants. In addition to the biopolymers, such as the abovementioned PLA, there a few natural polymers which also represent a promising class of materials with a wide range of applications, including use in implantable devices. These natural polymers include, collagen, hyaluronic acid, cellulose, chitosan, silk and others naturally derived proteins, as well as collagen, gelatin, albumin, elastin and milk proteins. These materials present certain advantages compared to the traditional materials (metals and ceramics) or synthetic polymers, such as biocompatibility, biodegradation and non-cytotoxicity, which make them ideal to be used in implantable drug delivery devices.

Dynamic or Active Polymeric Implants have a positive driving force to control the release of drugs from the device. The majority of the implants in this category are electronic systems made of metallic materials. Dynamic drug delivery implants are mainly pump type implants. The main type of polymeric active implants are osmotic pumps. This type of device is formed mainly by a semipermeable membrane that surrounds a drug reservoir. The membrane should have an orifice that will allow drug release. Osmotic gradients will allow a steady inflow of fluid within the implant. This process will lead to an increase in the pressure within the implant that will force drug release trough the orifice. This design allows constant drug release (zero order kinetics). This type of device allows a favorable release rate but the drug loading is limited.

In some embodiments, the factors are prepared as an injectable paste. The paste can be injected into the implant site. In some embodiments, the paste can be prepared prior to implantation and/or store the paste in the syringe at sub-ambient temperatures until needed. In some embodiments, application of the composite by injection can resemble a bone cement that can be used to join and hold bone fragments in place or to improve adhesion of, for example, a hip prosthesis, for replacement of damaged cartilage in joints, and the like. Implantation in a non-open surgical setting can also be performed.

In other embodiments the factors are prepared as formable putty. The hydrated graft putty can be prepared and molded to approximate any implant shape. The putty can then be pressed into place to fill a void in the cartilage, bone, tooth socket or other site. In some embodiments, graft putty can be used to repair defects in non-union bone or in other situations where the fracture, hole or void to be filled is large and requires a degree of mechanical integrity in the implant material to both fill the gap and retain its shape.

A system for pharmaceutical use, i.e. a drug delivery device with factors, can include, depending on the formulation desired, pharmaceutically-acceptable, non-toxic carriers of diluents, which are defined as vehicles commonly used to formulate pharmaceutical compositions for animal or human administration. The diluent is selected so as not to affect the biological activity of the combination. Examples of such diluents are distilled water, buffered water, physiological saline, PBS, Ringer's solution, dextrose solution, and Hank's solution. In addition, the NR pharmaceutical composition or formulation can include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents.

The composition can also include any of a variety of stabilizing agents, such as an antioxidant for example. When the pharmaceutical composition includes a polypeptide, the polypeptide can be complexed with various well-known compounds that enhance the in vivo stability of the polypeptide, or otherwise enhance its pharmacological properties (e.g., increase the half-life of the polypeptide, reduce its toxicity, enhance solubility or uptake). Examples of such modifications or complexing agents include sulfate, gluconate, citrate and phosphate. The polypeptides of a composition can also be complexed with molecules that enhance their in vivo attributes. Such molecules include, for example, carbohydrates, polyamines, amino acids, other peptides, ions (e.g., sodium, potassium, calcium, magnesium, manganese), and lipids.

Further guidance regarding formulations that are suitable for various types of administration can be found in Remington's Pharmaceutical Sciences, Mace Publishing Company, Philadelphia, Pa., 17th ed. (1985). For a brief review of methods for drug delivery, see, Langer, Science 249:1527-1533 (1990).

The pharmaceutical composition, i.e. combinations of factors and/or cells, can be administered for prophylactic and/or therapeutic treatments. Toxicity and therapeutic efficacy of the active ingredient can be determined according to standard pharmaceutical procedures in cell cultures and/or experimental animals, including, for example, determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit large therapeutic indices are preferred.

The data obtained from cell culture and/or animal studies can be used in formulating a range of dosages for humans. The dosage of the active ingredient typically lines within a range of circulating concentrations that include the ED50 with low toxicity. The dosage can vary within this range depending upon the dosage form employed and the route of administration utilized.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxin, which may be present during the synthesis or purification process. Compositions for parental administration are also sterile, substantially isotonic and made under GMP conditions.

The effective amount of a therapeutic composition to be given to a particular patient will depend on a variety of factors, several of which will differ from patient to patient. A competent clinician will be able to determine an effective amount of a therapeutic agent to administer to a patient to halt or reverse the progression the disease condition as required. Utilizing LD50 animal data, and other information available for the agent, a clinician can determine the maximum safe dose for an individual, depending on the route of administration. For instance, an intravenously administered dose may be more than an intrathecally administered dose, given the greater body of fluid into which the therapeutic composition is being administered. Similarly, compositions which are rapidly cleared from the body may be administered at higher doses, or in repeated doses, in order to maintain a therapeutic concentration. Utilizing ordinary skill, the competent clinician will be able to optimize the dosage of a particular therapeutic in the course of routine clinical trials.

### Methods of Treatment

The present invention provides methods of treating a bone lesion, or other injury in which growth of bone is desired, in an aged human or other animal subject, comprising applying to the site a composition comprising a combination of factors as set forth in the present disclosure, e.g. a combination of BMP2 and a CSF1 inhibitor. The factors can be provided in combinations with cements, gels, etc. As referred to herein such lesions include any condition involving skeletal tissue which is inadequate for physiological or cosmetic purposes. Such defects include those that are congenital, the result from disease or trauma, and consequent to surgical or other medical procedures. Such defects include for example, defect brought about during the course of surgery, dental implants, osteoarthritis, osteoporosis, infection, malignancy, developmental malformation, etc.

An individual in need of skeletal regeneration can be treated with the methods described herein. Various sites for bone regeneration can be treated, including without limitation ribs, lone bones, phalanges, facial bones, knee joint, elbow joint, joints in the phalanges and phalanxes, shoulder joints, hip joints, wrist joints, ankle joints, etc. The individual may be an adult, e.g. past adolescence, and may be an aged adult, e.g. a human over 55 years of age, over 60 years of age, over 65 years of age, over 70 years of age, etc.

At the time of injury, a drug delivery device is implanted or otherwise positioned to provide an effective dose of the combination of agents. The factors may be provided individually or as a single composition, that is, as a premixed composition of factors. The factors may be provided at the same molar ratio or at different molar ratios, e.g. where the ratio of BMP2 protein to anti-CSF1 is from about 1:20, 1:10, 1:5, 1:3, 1:2, 1:1, 2:1, 3:1, 5:1, 10:1, 20:1, etc. on a wt/wt basis. The factors may be provided once or multiple times in the course of treatment. For example, an implant comprising factors may be provided to an individual, and additional factors and/or cells provided during the course of treatment.

While in many cases the endogenous SSC are sufficient for regeneration, optionally exogenous cells are provided at the site of local acute injury. The cells may be SSC, or non-SSC, e.g. mesenchymal stem cells, adipose stem cells, etc. The cells may be autologous or allogeneic. The cells may be provided concomitant with the provision of growth factors, e.g. simultaneously, shortly before, shortly, after, etc. and may be in a single implant with the growth factors, as a separate implant or injection, etc.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

Aged Skeletal Stem Cells Generate an Inflammatory Niche that Impedes Skeletal Integrity.

Skeletal aging and disease are associated with a misbalance in the opposing actions of osteoblasts and osteoclasts that are responsible for maintaining the integrity of bone tissues. Here, we show through detailed functional and single-cell genomic studies that intrinsic aging of bona fide mouse skeletal stem cells (SSCs) alters bone marrow niche signaling and skews bone and blood lineage differentiation leading to fragile bones that regenerate poorly. Aged SSCs have diminished bone and cartilage forming potential but produce higher frequencies of stromal lineages that express high levels of pro-inflammatory and pro-resorptive cytokines. Single-cell transcriptomic studies tie the functional loss to a diminished transcriptomic diversity of SSCs in aged mice thereby contributing to bone marrow niche transformation. While systemic exposure to a youthful circulation through heterochronic parabiosis reduced inflammation and myelopoiesis, it did not reverse the diminished osteochondrogenic activity of aged SSCs and was insufficient to improve bone mass and skeletal healing parameters in aged mice. Hematopoietic reconstitution of aged mice with young hematopoietic stem cells (HSC) also did not improve bone integrity and repair. Conversely, the aged SSC lineage promoted myeloid skewing suggesting SSC-derived cells as a unidirectional driver of hematopoietic aging. We found that deficient bone regeneration in aged mice could only be reversed by the local application of a combinatorial treatment that re-activates aged SSCs and simultaneously abates crosstalk to hematopoietic cells favoring an inflammatory milieu. This treatment expanded a functional SSC pool, tempered osteoclastogenesis, and enhanced bone healing to youthful levels. Our findings provide mechanistic insight into the complex, multifactorial mechanisms underlying skeletal aging and offer new prospects for rejuvenating the aged skeletal system.

### Results

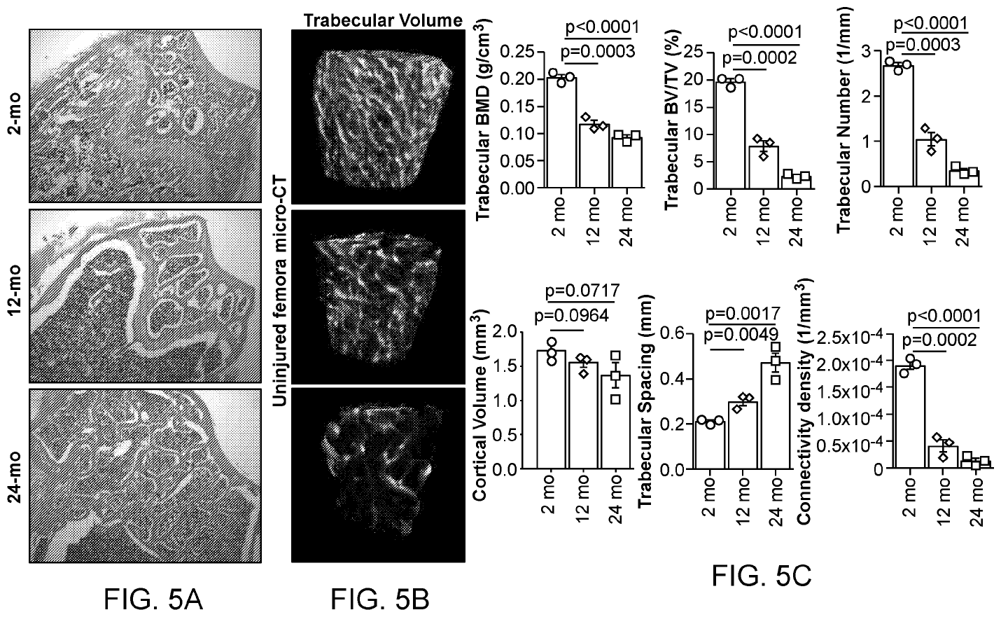

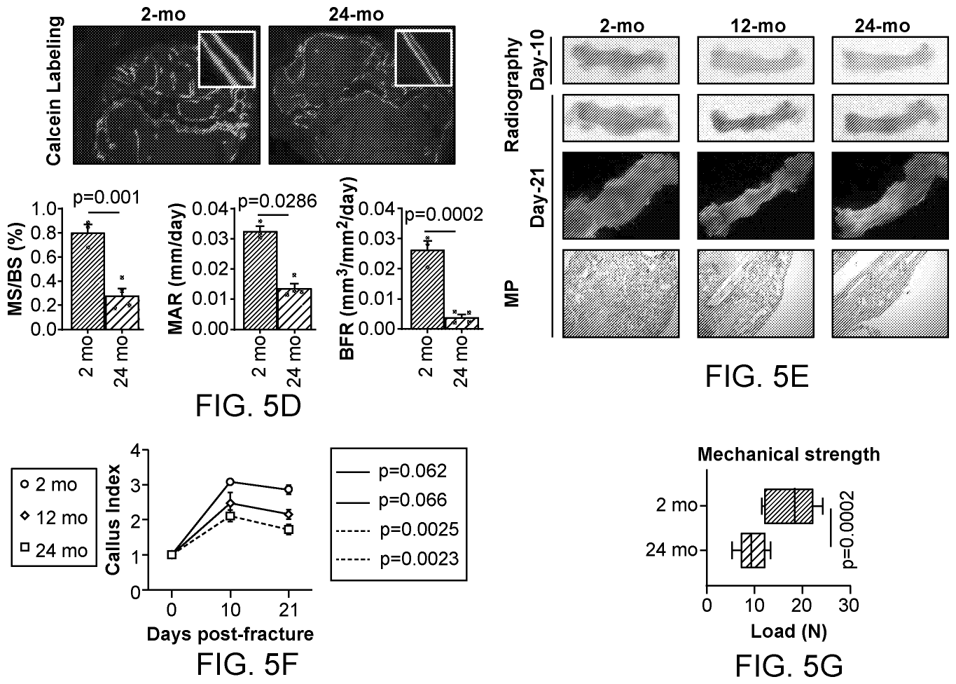

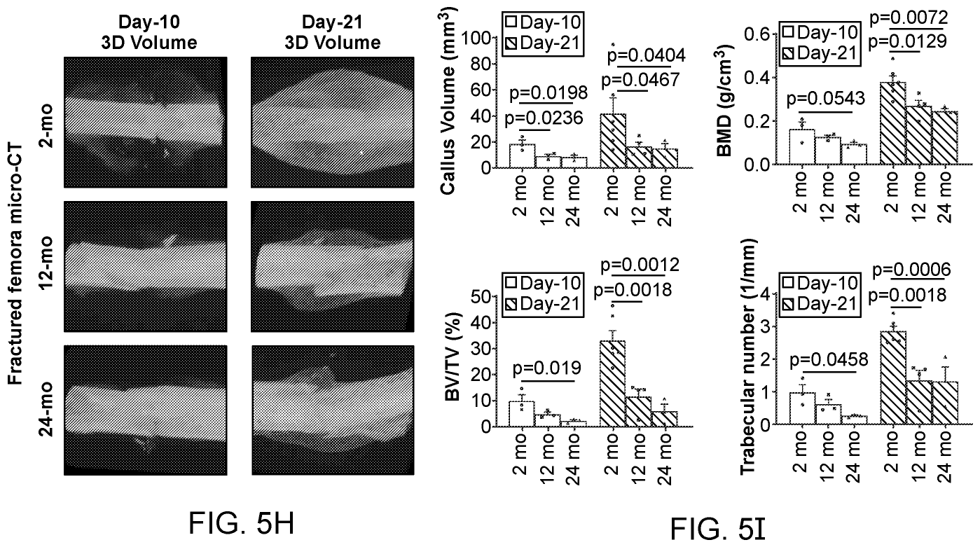

Age-related bone loss and regenerative decline coincide with a diminished skeletal stem cell pool with skewed lineage output. In agreement with previous reports, we observed an age-dependent decline in skeletal homeostasis between 2-month-old (‘2-mo’) and 24-month-old (′24-mo′) C57BL/6J male mice. This aged skeletal phenotype was associated with distinct changes in bone architecture, including an attenuation of the growth plate, reduced bone mineral density (BMD), and decreased trabecular bone mass (FIG. 5a-c). In addition, it was notable for a significant decrease in active matrix mineralization via calcein labeling, an in vivo measurement of bone formation and remodeling (FIG. 5d). Using a transverse mid-diaphyseal femoral fracture model, we also observed differences in the skeletal regeneration capacities of aging mice. Radiographic and histologic analyses of healing femora revealed that ‘24-mo’ mice formed significantly smaller calluses, and mechanical strength testing showed that these calluses were more prone to re-fracture at post-fracture day-21 (FIG. 5e-g). Micro-CT measurements at multiple post-fracture timepoints confirmed that ‘24-mo’ calluses had less volume and were significantly less mineralized (FIGS. 5h-i). Taken together, these data indicate that the aged skeletal phenotype is characterized by a decline in both homeostatic and regenerative bone formation.

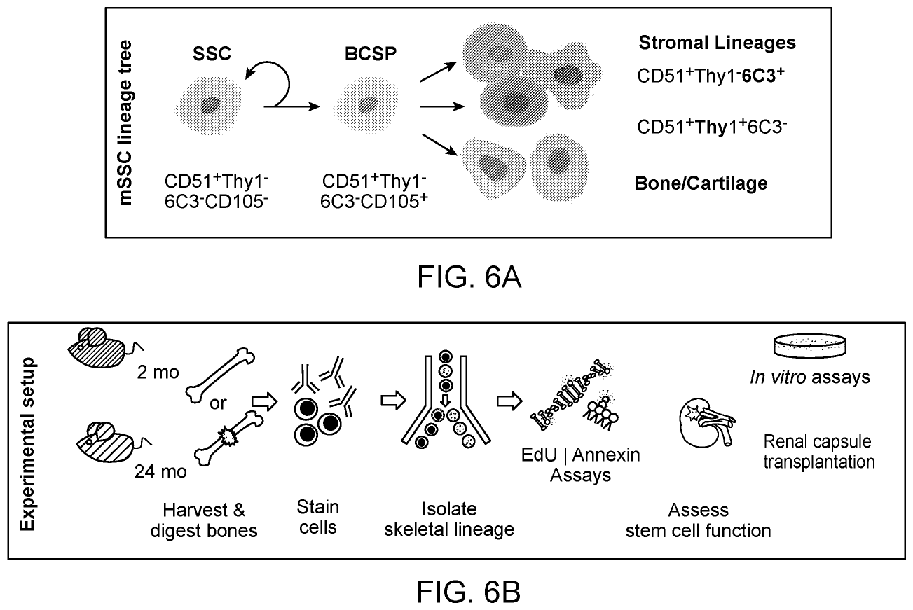

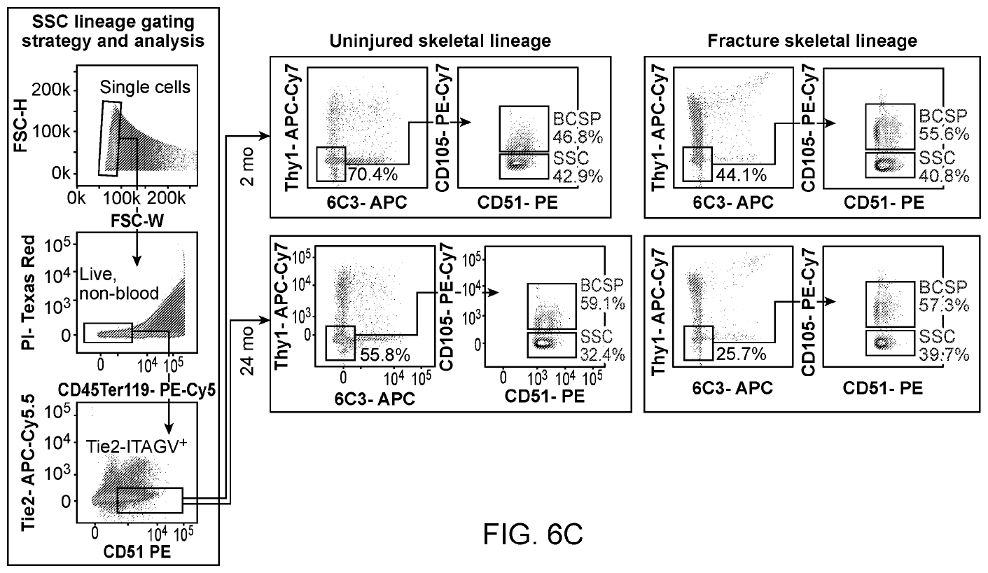

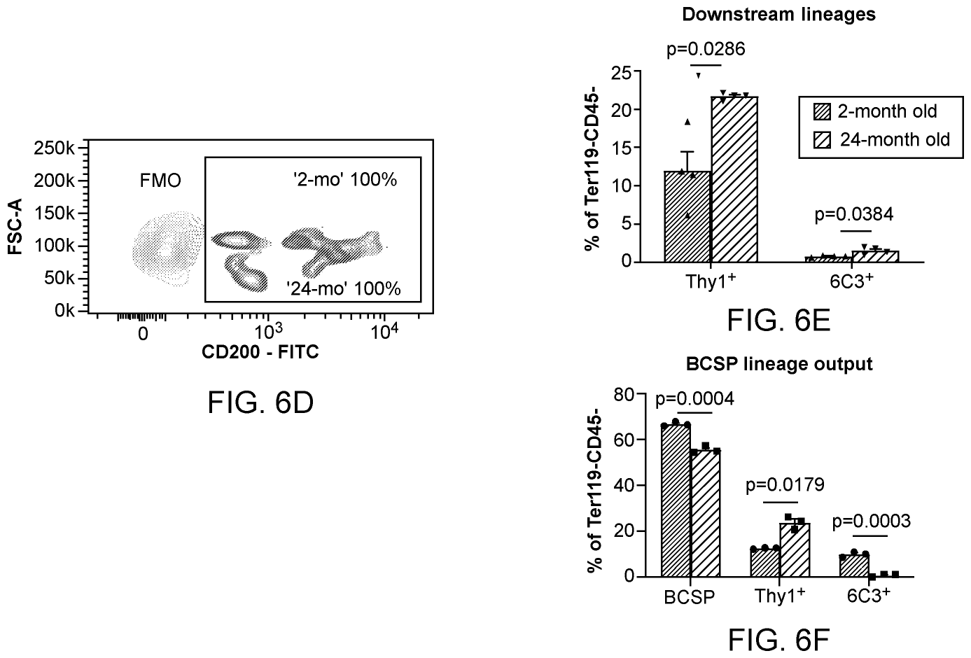

Many postnatal organs are maintained and repaired by tissue-resident stem cells and emerging evidence suggests that age-related decline in tissue function are due to the loss of stem cell activity. To determine if reduced stem cell activity, including proliferation and differentiation capacity, could be responsible for the aged skeletal phenotype in mice, we next focused on the cellular origins of bone-forming cell types. In previous studies we described a purified, self-renewing mouse and human skeletal stem cell (SSC) that, through a multipotent bone-cartilage-stromal progenitor (BCSP), gives rise to committed lineage cells of bone, cartilage, and stroma but not fat (FIG. 6a-c). The stromal cell populations are further delineated by a Thy1+ subset, which supports short-term hematopoietic progenitors and myeloid cell populations in vitro, and a 6C3+ subset, which maintains lymphopoiesis and hematopoietic stem cells (HSCs). We also observed that in contrast to fetal SSCs, in the adult SSC lineage all CD51⁺Thy1⁻6C3⁻CD105⁻ cells were CD200-positive, not allowing further separation into SSCs and pre-BCSPs (FIG. 6d). Given earlier reports showing the role of SSCs in skeletal maintenance and regeneration, it seemed reasonable that declining SSC activity underlies the aged skeletal phenotype.

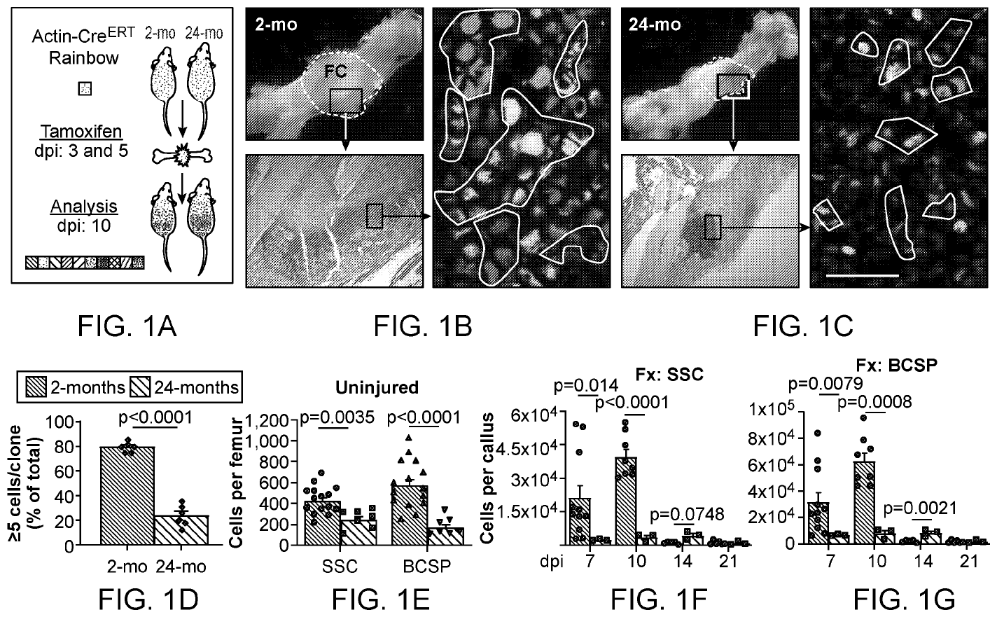

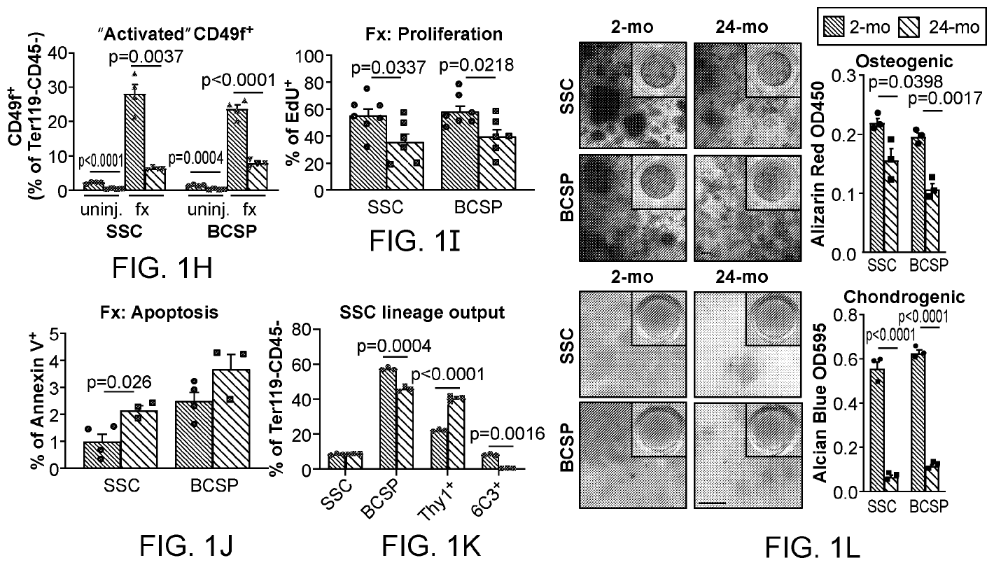

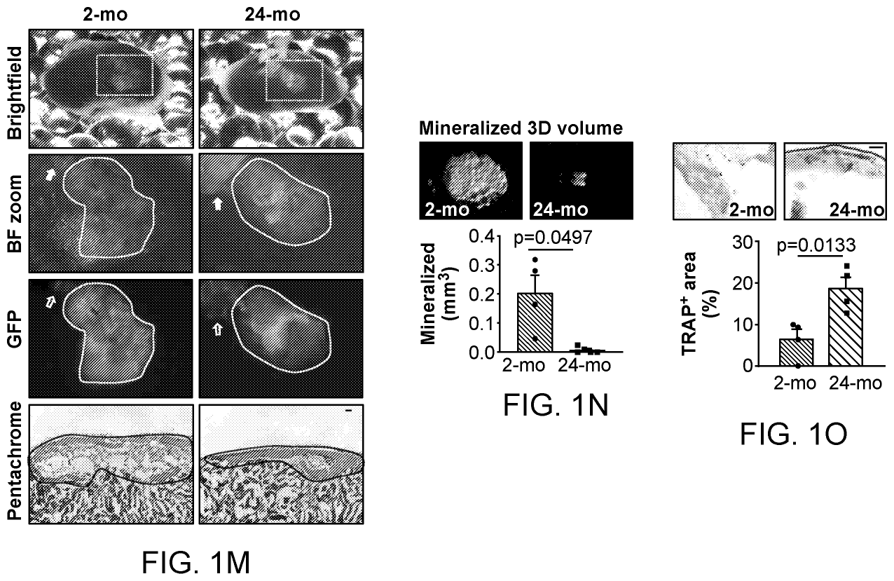

Previously, we demonstrated location of clonal skeletogenesis, consistent with the detection of SSCs by fluorescence-activated cell sorting (FACS) and the presence of stem cell activity, in the growth plates of postnatal day 3 long bones using Actin-Cre^{ERT} Rainbow (“Rainbow”) mice, which harbor a tamoxifen-inducible, ubiquitously expressed Cre under the Actin promoter. Here, we used the Rainbow model in the setting of skeletal regeneration, known to stimulate SSC activity, to compare clonal activity in ‘2-mo’ versus ‘24-mo’ calluses. Transverse femoral fractures were created, and mice were treated with tamoxifen on post-injury days 3 and 5 to activate Cre recombinase (FIG. 1a). We found significantly increased clonal activity with clones containing a higher number of progenies (>5 cells per clone) in healing calluses of 2-mo mice compared to ‘24-mo’ mice at post-fracture day-10 (FIG. 1b-d).

We then isolated SSCs and BCSPs from uninjured and fractured bones using FACS and observed corresponding reductions in total cell numbers with age (FIG. 1e-g). In addition, the prevalence and clonal activity of CD49f-expressing SSCs and BCSPs, which have been shown to exhibit greater osteogenic capacity than steady state SSCs and BCSPs, were reduced significantly in 24-mo calluses (FIG. 1h). These changes were associated with significantly decreased proliferation, whereas apoptosis was generally low but increased slightly in ‘24-mo’ mice (FIG. 1i-j). Finally, flow cytometric analyses revealed that downstream SSC lineage output was shifted towards pro-myeloid Thy1+ and away from 6C3+ stroma in ‘24-mo’ mice both during in vitro culture and when induced by fracture injury in vivo (FIG. 1k & FIGS. 6e-f). Altogether, these data suggest that the aged skeletal phenotype corresponds to reduced SSC activity and shifted SSC lineage output in mice.

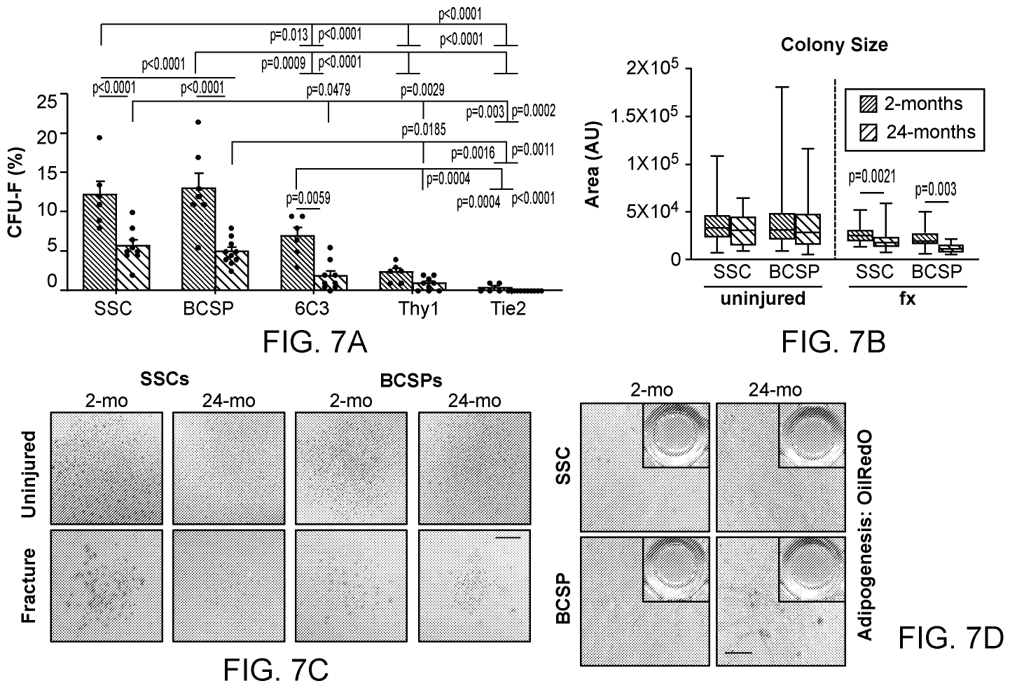

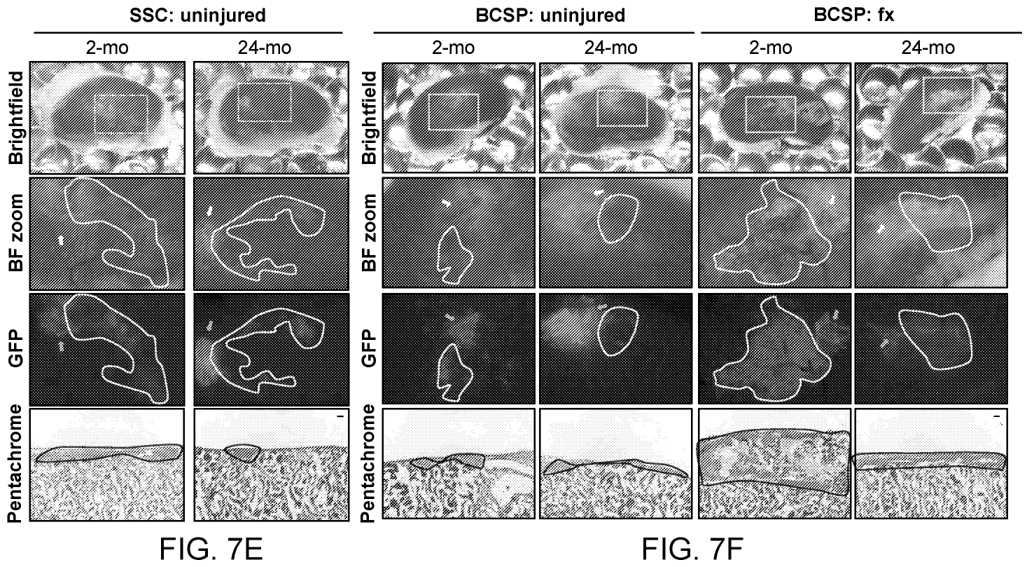

Mouse SSCs display intrinsic aging. We next isolated SSCs and BCSPs from ‘2-mo’ and ‘24-mo’ mice to compare their intrinsic ability to generate skeletal tissue in vitro and in vivo. SSCs and BCSPs displayed significantly higher colony-forming ability than downstream cell populations independent of age suggesting that surface marker profiles of SSCs/BCSPs are preserved in ‘24-mo’ mice (FIG. 7a). Consistent with our observations in Rainbow mice (FIGS. 1a-d), SSCs and BCSPs isolated from 24-mo bone generated fewer and smaller colonies in vitro (FIGS. 7b-c). They also had reduced osteogenic capacity as well as significantly reduced chondrogenic capacity in vitro, in agreement with previous reports that long bone SSCs form bone through a cartilaginous intermediate (FIG. 1l). Notably, we also observed that neither young nor aged SSCs undergo adipogenic differentiation, even under highly adipogenesis-inducing conditions in vitro (FIG. 7d).

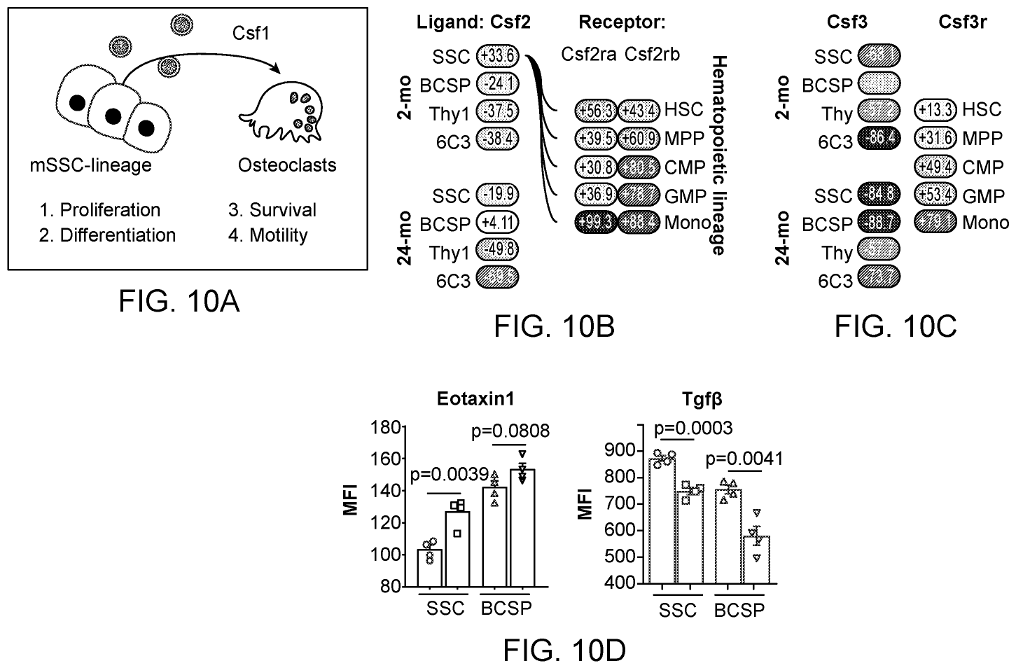

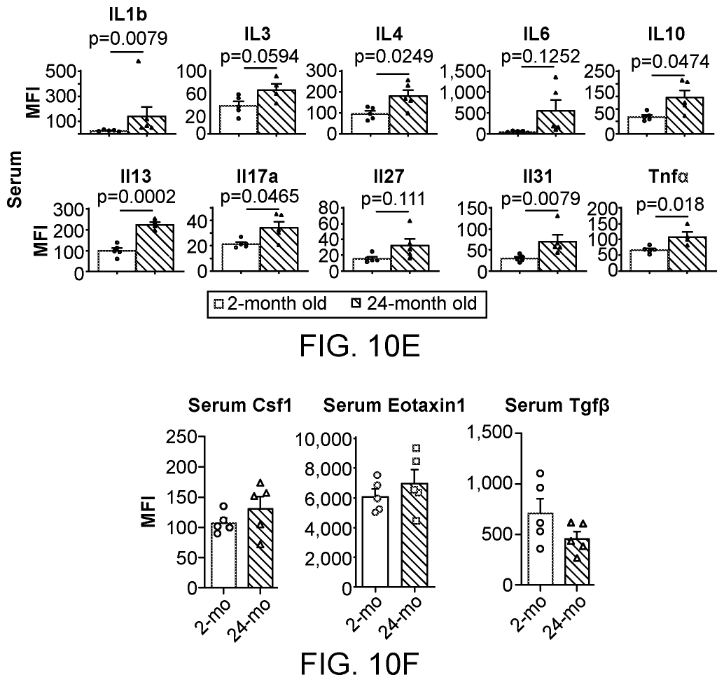

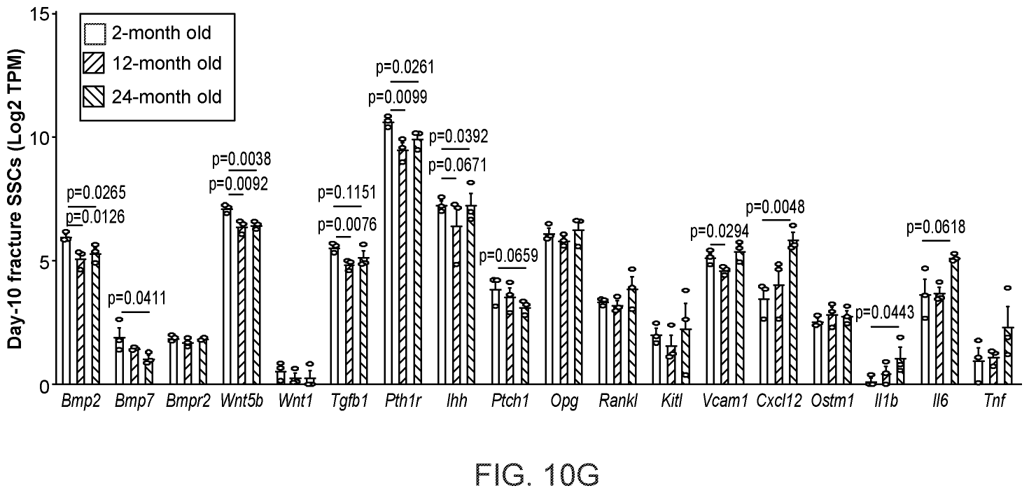

To study intrinsic skeletogenic potential in vivo, we purified 3,000 GFP-labeled SSCs and BCSPs from bones of ‘2-mo’ and ‘24-mo’ mice with or without fracture using FACS and transplanted them beneath the renal capsules of ‘2-mo’ immunocompromised NSG mice. After four weeks, histological and μCT analyses showed that ‘2-mo’ SSCs and BCSPs formed robust bone grafts with an ectopic marrow cavity. In contrast, ‘24-mo’ SSCs and BCSPs formed smaller and less mineralized grafts (FIGS. 1m-n & FIGS. 7e-f). Consistent with prior studies, SSCs and BCSPs isolated from fractured bone formed visually larger ossicles than those isolated from uninjured bones (FIGS. 7e-f). However, aging decreases the skeletogenic potential of SSCs, even when isolated from a fracture environment. Furthermore, staining grafts for osteoclast activity by tartrate-resistant acid phosphatase (TRAP) showed more bone resorption in aged grafts, implying that aged SSCs create grafts that stimulate increased osteoclast activity (FIG. 10). In summary, transplantation of aged SSCs and BCSPs into young mice did not improve their skeletogenic potential, indicating that cell-autonomous changes underlie their functional decline and suggesting that aged skeletal lineages produce factors that enhance bone resorption.

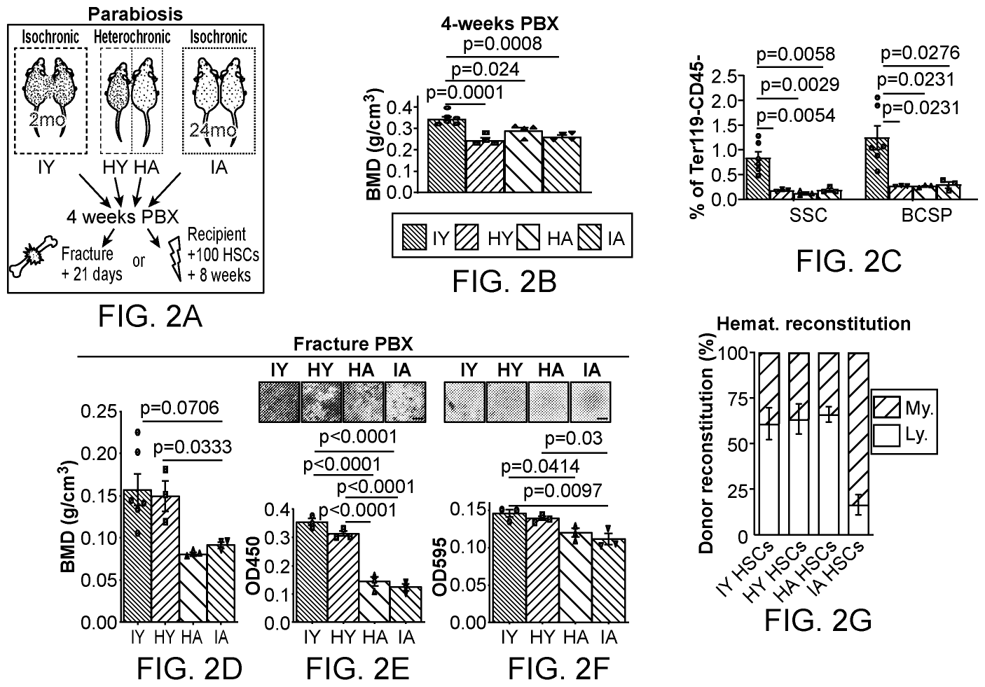

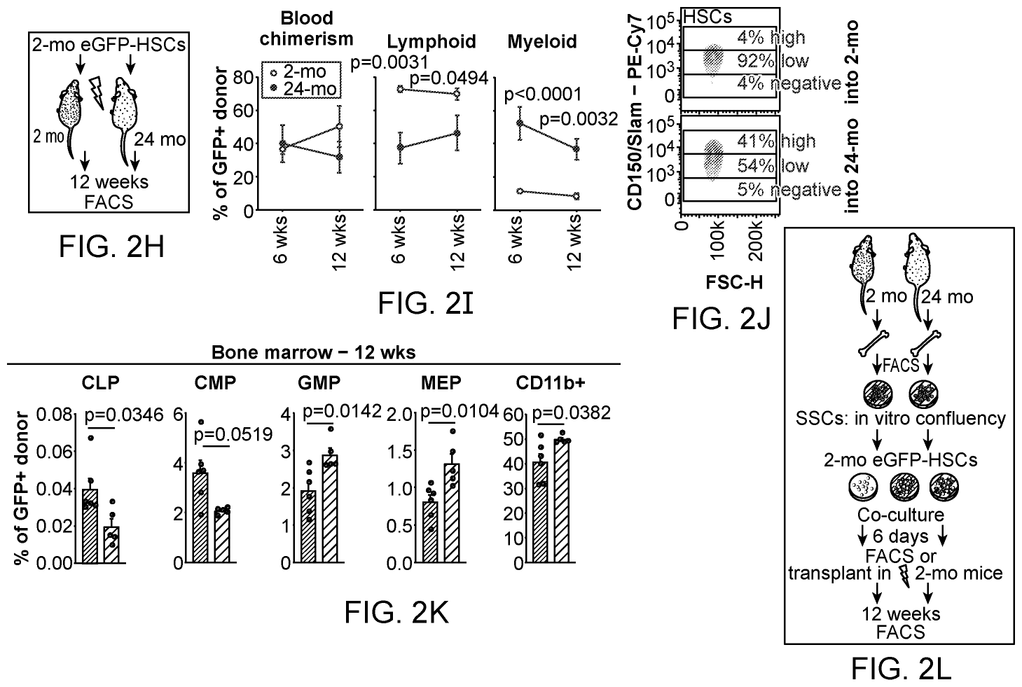

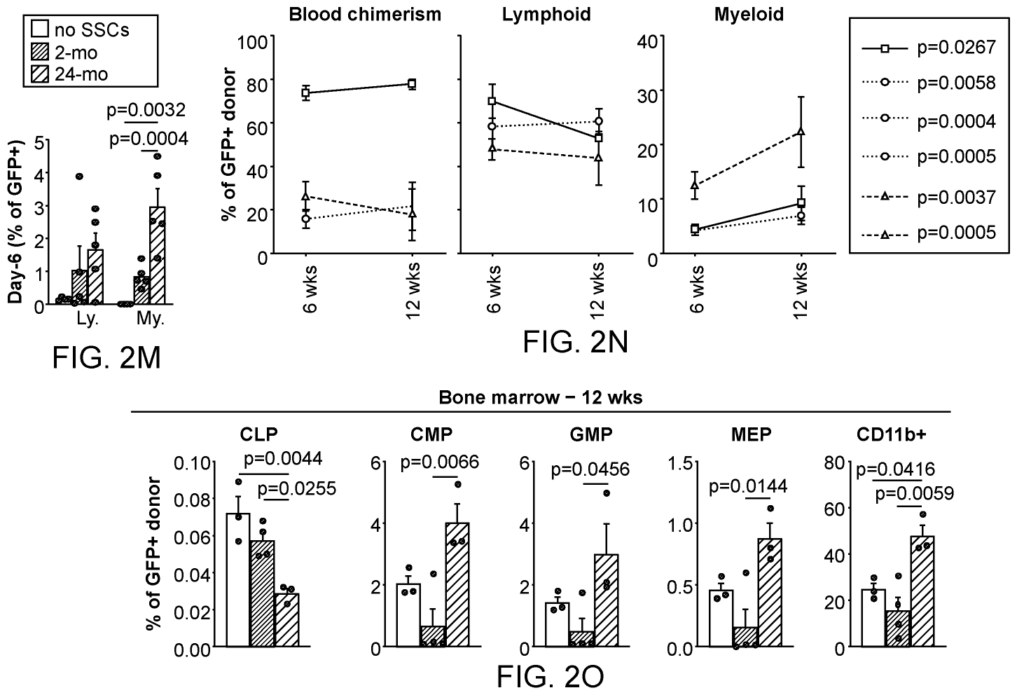

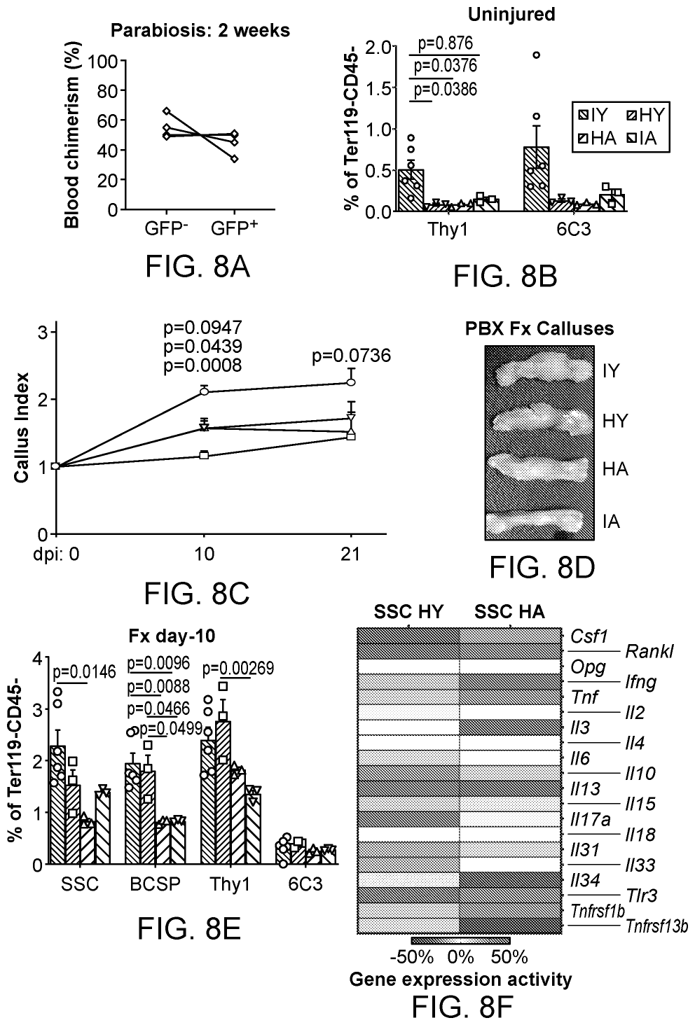

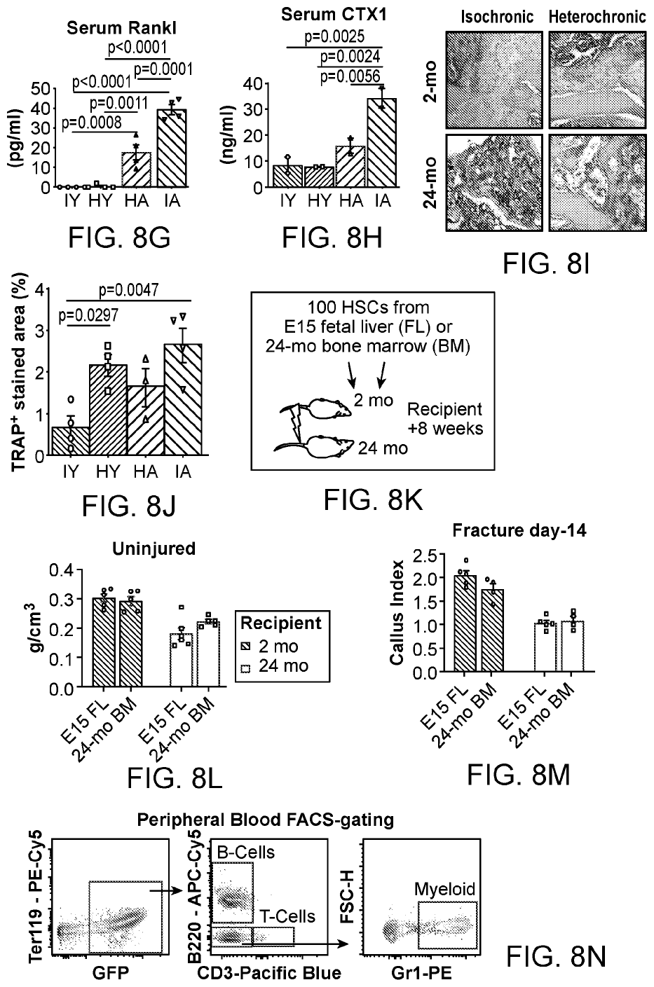

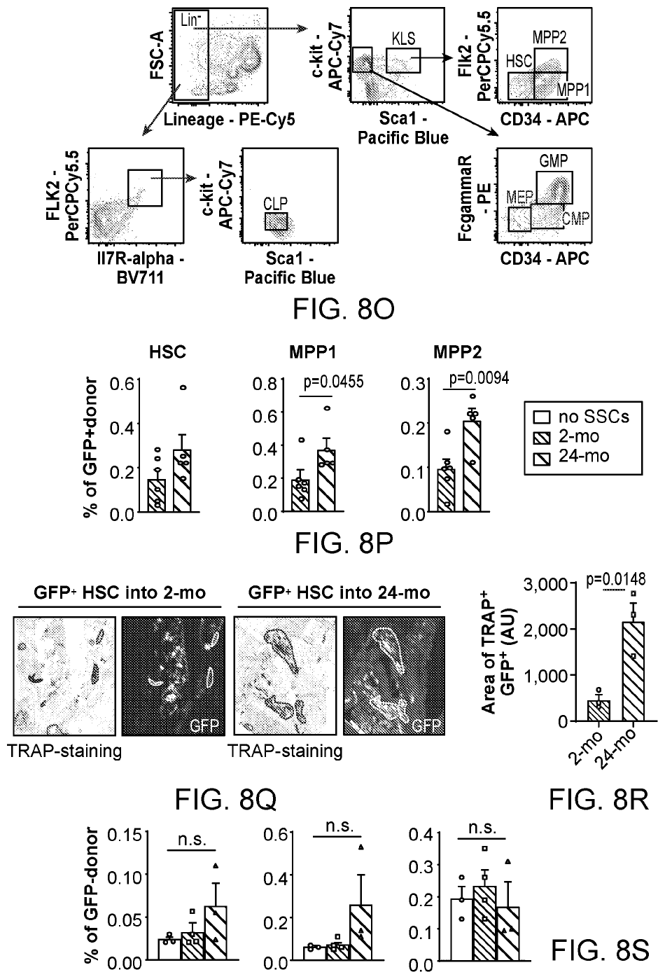

Exposure to youthful blood does not restore SSC function and skeletal parameters. Prior studies suggest that exposure to young blood can reverse age-related changes in skeletal muscle, heart, and brain tissue. Because heterotopic transplantation into a young host did not rejuvenate aged SSC activity, we wondered if exposure to a young systemic circulation by heterochronic parabiosis could revive SSCs within their endogenous microenvironment. Previously, we have shown that SSCs do not migrate systemically; therefore, differences in skeletogenic activity between parabionts are likely due to effects of circulating factors. To test this hypothesis, we generated heterochronic pairs in which ‘2-mo’ and ‘24-mo’ mice were joined surgically. Isochronic pairs of ‘2-mo’ and ‘24-mo’ mice were created for comparison (FIG. 2a). We confirmed peripheral blood chimerism in two-week parabionts (FIG. 8a). At week four of parabiosis, μCT measurements showed that the BMD of femora from heterochronic aged (HA) mice remained significantly lower than those from isochronic young (IY) mice (FIG. 2b). In contrast, exposure to aged blood significantly reduced the BMD of heterochronic young (HY) femora compared to IY controls. Heterochronic parabiosis also significantly reduced the frequency of skeletal lineage cells in both parabionts when compared to IY controls, while no significant difference in skeletal lineage cell frequency between heterochronic and IA mice was observed (FIG. 2c & FIG. 8b).

Next, to test the effect of heterochronic parabiosis on skeletal regeneration, we created transverse femoral fractures in each parabiont four weeks after parabiosis. We found that while callus size, an indicator of healing response, initially increased in HA and decreased in HY parabionts, no significant changes were observed at post-injury day-21 (FIG. 8c-d). Furthermore, the BMD of healing calluses harvested from HA mice did not improve from IA controls (FIG. 2d). SSC/BCSP frequencies also did not significantly improve in healing femora of HA parabionts compared to isochronic controls (FIG. 8e). In addition, the in vitro osteogenic and chondrogenic potentials of SSCs isolated from HA calluses remained similar to IA derived SSCs and significantly decreased compared to IY SSCs (FIG. 2e-f). Interestingly, both the BMD and SSC activity of healing femora calluses harvested from HY mice remained unaffected by exposure to aged blood, in contrast to our findings in uninjured HY femora (FIG. 2d-f & FIG. 8e). Collectively, these results indicate that factors present in the circulation of aged mice can exert a negative effect on the young SSC lineage during steady state heterochronic parabiosis. SSC aging might therefore be the consequence of a priming event that institutes an intrinsically aged state which renders these cells henceforth insensitive to physiologic concentrations of blood-borne factors.

Myeloid lineage overproduction is a hallmark of HSC aging and has been attributed to an age-related overabundance of pro-inflammatory cytokines. In the context of heterochronic parabiosis, expression of pro-inflammatory marker genes by purified SSCs was low in both age groups (FIG. 8f). Serum levels of pro-myogenic, osteoclastic Rankl and the bone resorption marker Ctx1 were reduced in HA compared to IA mice, however, osteoclastic activity at fracture sites was elevated for HY compared to IY mice (FIG. 8g-j), suggesting the possibility of an altered local microenvironment, at least in part through changes in SSC lineage composition and the involvement of factors beyond Rankl. Surprisingly, phenotypic HSCs derived from each parabiont, transplanted into lethally irradiated young recipients showed balanced lineage output for IY, HY, and HA derived cells, whereas IA HSCs gave rise to mostly myeloid lineages (FIG. 2g). These findings indicate that, unlike in SSCs, exposure to young blood-borne factors was able to reprogram aged HSCs to function like young HSCs, although the presence of circulating young HSCs in HA mice cannot be excluded.

Aged SSC lineage cells promote myeloid-biased HSC output. Based on these findings, we next tested the possibility of unidirectional crosstalk from SSC to HSC lineage cells. Transplantation of lymphoid-biased fetal liver derived HSCs did not improve BMD or fracture healing in ‘2-mo’ or ‘24-mo’ recipients when compared to transplantation of myeloid-biased 24-mo mouse HSCs (FIG. 8k-m). Alternatively, lethally irradiated ‘2-mo’ or ‘24-mo’ mice reconstituted with ‘2-mo’ GFP-labeled HSCs showed comparable chimerism but significant variations in donor-derived peripheral blood composition at 12-weeks. In particular, ‘24-mo’ mice presented with a myeloid bias at the expense of lymphoid output (FIG. 2h-i and FIG. 8n). Bone marrow analyses revealed that donor-derived Lin−Sca1+ckit+Flt3−CD34− HSCs in ‘24-mo’ mice contained larger proportions of myeloid biased CD150/Slam^{high} expressing cells compared to ‘2-mo’ mice (FIG. 2j and FIG. 80-p)⁹. In line with that observation, at 12-weeks frequencies of engrafted bone marrow cells in ‘24-mo’ mice significantly shifted from generating Common Lymphoid Progenitors (CLPs) toward producing myeloid lineages, including Megakaryocyte-Erythroid Progenitors (MEPs), Granulo-Monocytic Progenitors (GMPs), and CD11b-expressing monocytic cells (FIG. 2k and FIG. 8p). In support of these results, we found that if we induced bi-cortical fractures in the same animals, significantly more donor-derived TRAP-positive osteoclastic areas were observed at day-10 callus sites of 24-mo mice compared to 2-mo old mice (FIG. 8q-r). To study crosstalk between skeletal and hematopoietic lineages, we FACS-isolated SSCs from 2-mo and 24-mo mice, expanded them to confluency, and co-cultured each group with 1,000 purified GFP-labeled HSCs (FIG. 2l). After six days of co-culture, we analyzed GFP-positive cells and found that ‘24-mo’ SSCs produced predominantly myeloid output by co-cultured HSCs compared to ‘2-mo’ SSCs. Strikingly, HSCs cultured alone remained largely uncommitted (FIG. 2l). When these co-cultures were then transplanted into lethally irradiated 2-mo recipient mice, hematopoietic cells that had originally been exposed to ‘24-mo’ SSCs continued to promote myeloid expansion in peripheral blood (FIG. 2n), as well as higher CMP, GMP, MEP, and CD11b+ output at the expense of CLP populations (FIG. 20 and FIG. 8s). Taken together, these findings suggest that while young HSCs were unable to re-establish youthful skeletal physiology, aged SSC lineage cells directly contribute to the age-related functional alterations of the hematopoietic system.

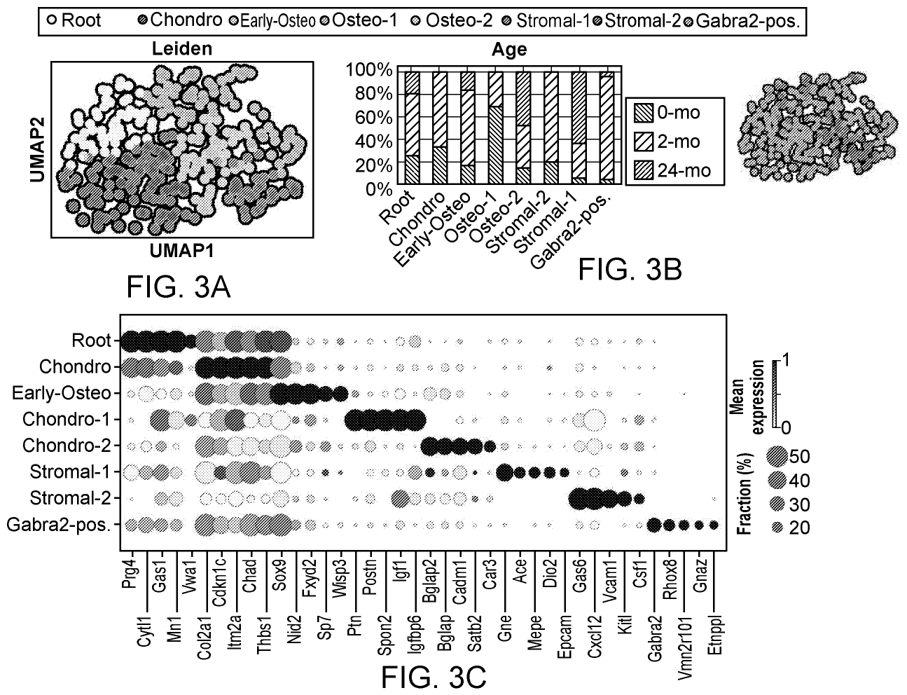

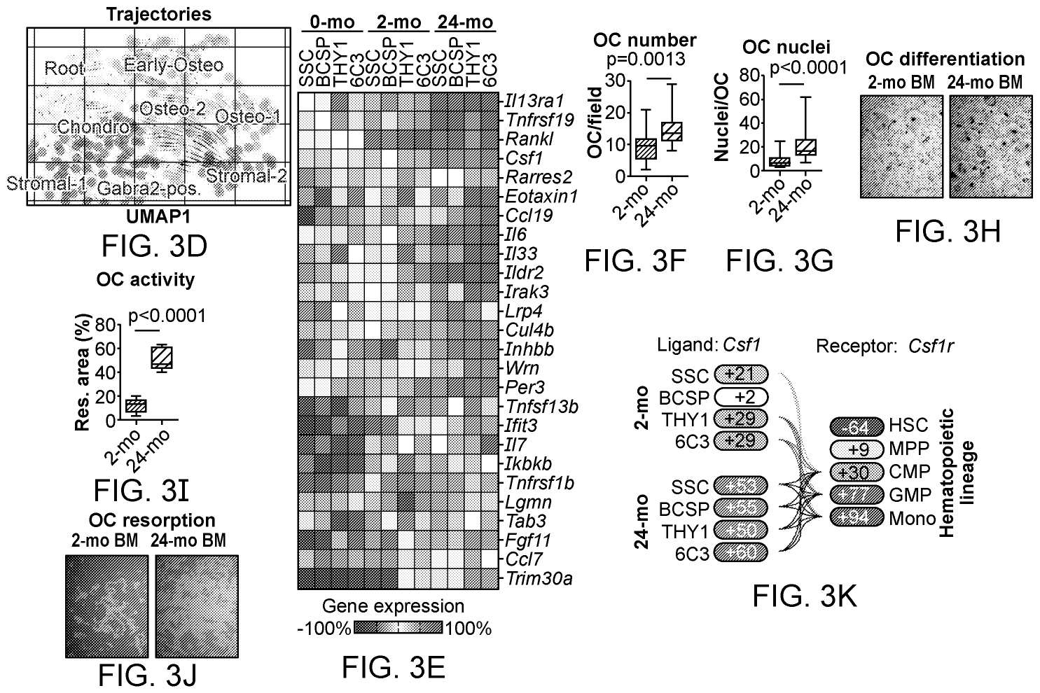

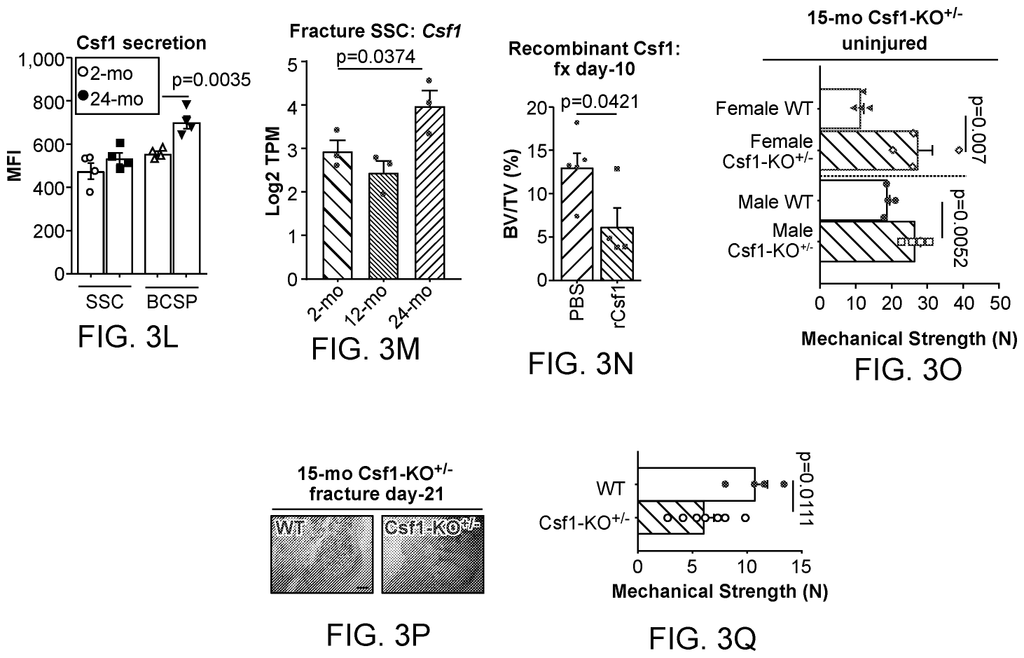

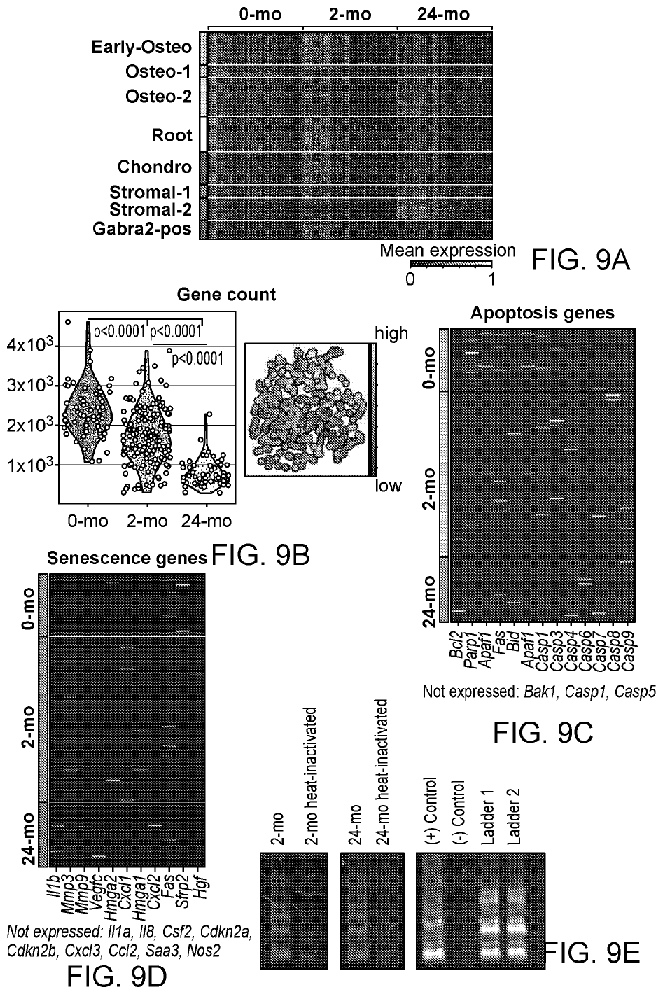

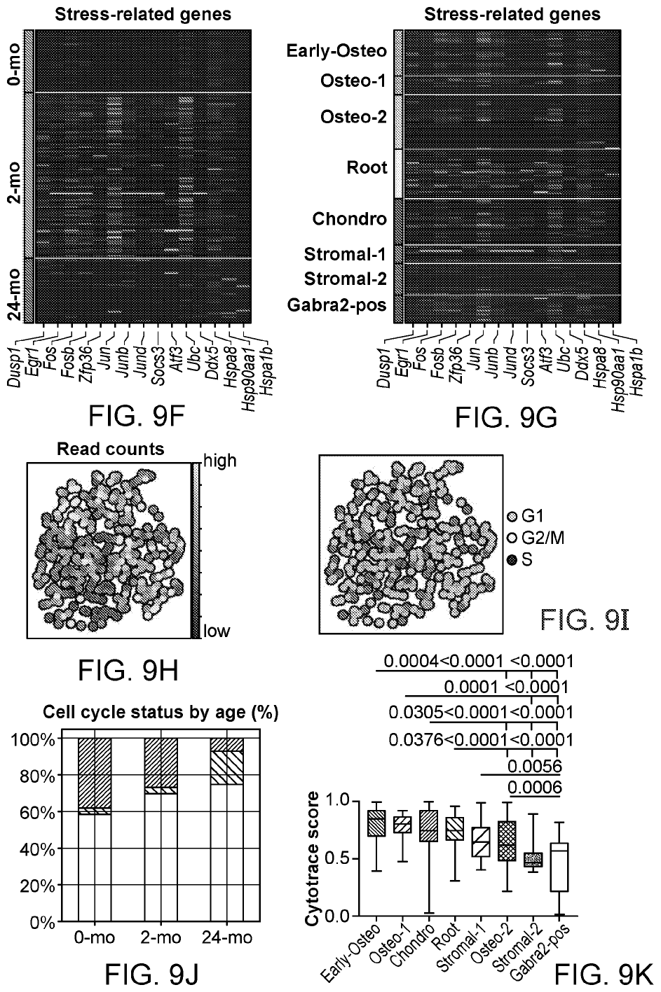

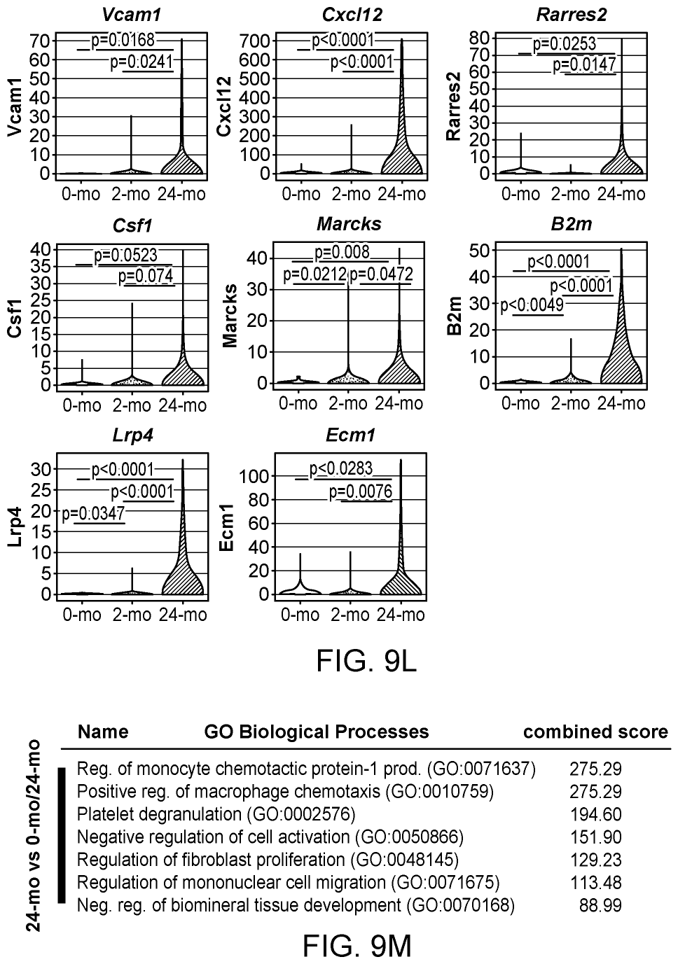

Single-cell transcriptomic profiling reveals the molecular basis of SSC aging. In many tissues, age-dependent physiological decline is thought to be preordained at the stem cell level. This paradigm is supported by HSC studies that show that aging is reversible via reprogramming techniques that target specific age-related stem cell programs. To determine if SSC aging is also associated with characteristic transcriptional changes, we FACS purified phenotypic SSCs from the long bones of postnatal day-3 (‘0-mo’), ‘2-mo’, and ‘24-mo’ mice for full-length SmartSeq2 single-cell RNA-sequencing (scRNAseq). Analysis of filtered high quality single cells revealed a high transcriptomic similarity of top expressed genes between SSCs of different age groups despite significantly lower total gene counts in 24-mo SSCs (FIG. 3a-b & FIG. 9a-b). Analyzed cells showed no notable expression of genes related to apoptosis or senescence across all cells, in line with unaltered telomerase activity between 2-mo and 24-mo SSCs (FIG. 9c-e). Of note, as reported for tissue dissociation protocols, stress-induced gene expression was observed in single SSCs, albeit limited to cells from ‘2-mo’ mice (FIG. 9f). This might point at a more sensitive environmental sensing of ‘2-mo’ SSCs that in turn at least to some extent explains the strong responses of the same group when exposed to aged blood during parabiosis experiments.

To explore heterogeneity of SSCs we performed Leiden clustering (FIG. 3a-b & Supplementary Table 1). Expression of stress-associated genes, total read count per cell, and cell cycle status had no dominant effect on cluster distribution (FIG. 9g-j). We identified eight subpopulations with subtle differences in their transcriptomic profiles, indicative of potential priming stages of distinct SSC subsets (FIG. 3c). Based on enriched genes these clusters were annotated as a chondrogenic ‘Chondro’ (Col2a1, Chad, Itm2a) population, osteogenic, separated into ‘Early-Osteo’ (Sox9, Nid2, Sp7), ‘Osteo-1’ (Ptn, Postn, Igf1) and ‘Osteo-2’ (Bglap, Cadm1, Car3) cell types, and stromal, with extracellular matrix genes-expressing ‘Stromal-1’ (Gne, Ace, Epcam) and pro-hematopoietic ‘Stromal-2’ (Gas6, Cxcl12, Csf1) cells as well as a ‘Gabra2-positive’ (Gabra2) population that warrants further investigation in the future (FIG. 3a-c). By performing Cyto Trace analysis to detect variations in differentiation state and Velocyto to infer trajectories thereof we could annotate the remaining cluster as the potential SSC ‘Root’ (Prg4, Cytl1, Mn1) (FIG. 3d & FIG. 9k). Accordingly, SSCs from all age groups were represented in the ‘Root’ cluster. While young ‘0-mo’ and ‘2-mo’ SSCs were part of each cluster, they were specific to ‘Chondro’, ‘Osteo-1’, and ‘Stromal-1’ implying multiple distinct fate specification programs at young ages. In contrast, ‘24-mo’ SSCs were most abundant in the pro-hematopoietic ‘Stromal-2’ cluster which altogether suggested shifted and limited transcriptionally determined lineage dynamics during aging, e.g., explaining the strongly reduced chondrogenic differentiation and in vivo ossicle formation capacity (FIG. 1l-n). In sum, these results couple the functional decline of aged SSCs to a loss in transcriptomic diversity thereby restricting developmental potential.

When we further probed specific differences in gene expression as a consequence of aging we found that 24-mo SSCs were enriched for genes that have been associated with reduced bone formation (Lrp4, Ecm1) and increased bone resorption (B2m, Rarres2, Marcks, Csf1) (FIG. 9l). Gene ontology analysis of biological processes based on differential expression between 24-mo and young age groups additionally revealed an enrichment of genes associated with increased monocyte/macrophage chemotaxis, supporting the pro-myeloid axis observed in functional experiments (FIG. 2 & FIG. 9m). Altogether, these findings provide a molecular basis by which aged SSCs lose their skeletogenic potential while also increasing their interaction with the hematopoietic compartment which ultimately could promote enhanced osteoclastogenesis.

The aged SSC lineage generates a pro-osteoclastogenic niche through expression of Csf1. Due to the relative rarity of stem cells and that stem-cell based defects are likely inherited by their progenies, we reasoned that we would find similar gene expression alterations in downstream SSC lineages. Thus, we extended our transcriptomic analyses to include detailed genome-wide microarray data of ‘0-mo’, ‘2-mo’, and ‘24-mo’ SSCs, BCSPs, Thy1+, and 6C3+ cells. Results indeed showed a pronounced age-related shift in all lineage populations to a profile supportive of inflammatory osteoclastogenesis (FIG. 3e). Interestingly, Rankl and Csf1, factors necessary and sufficient for osteoclast maturation⁴⁰, showed specific expression patterns (FIG. 10a). While Rankl was expressed at relative high levels at ‘2-mo’ and ‘24-mo’, Csf1 displayed a pronounced increase in all four subpopulations in 24-mo mice. To test functionally if aged bones are more responsive to these two pro-osteoclastic factors, we supplemented whole bone marrow from ‘2-mo’ and ‘24-mo’ mice with Rankl and Csf1 in vitro. We found that ‘24-mo’ bone marrow produced more and larger osteoclasts, and these osteoclasts showed enhanced resorption activity compared to those that were differentiated from ‘2-mo’ bone marrow (FIG. 3f-j). We also found that SSC-lineage specific Csf1 expression matched with strong Csf1r on GMP and monocytes, the hematopoietic cell types that increased in frequency when aged SSCs were co-cultured with HSCs (FIG. 2n-o, 3k and FIG. 10b-c). Based on these combined observations and previous reports showing that ovariectomy-induced bone loss can be prevented by neutralizing Csf1, we considered Csf1 as a specific therapeutic target to counteract the aged SSC lineage pro-myeloid secretome.

We then quantified Csf1 secretion by FACS-purified SSCs and BCSPs in vitro and found that 24-mo cells secreted increased levels of Csf1 as determined by Luminex assay (FIG. 31). At the same time, ‘24-mo’ cells were also found to secrete more pro-inflammatory Ccl11 (Eotaxin1) and less pro-osteochondrogenic Tgfβ (FIG. 10d). Next, we studied the systemic inflammatory states of ‘2-mo’ and ‘24-mo’ mice via Luminex assay and found a general increase in inflammatory factors (e.g., Il1b, Il10, Tnfa) in serum of aged mice (FIG. 10e). However, systemic levels of Csf1, Ccl11, and Tgfβ were not significantly different between ‘2-mo’ and ‘24-mo’ mice, suggesting that protein secretion by the SSC lineage was restricted to the local niche environment (FIG. 10f). Moreover, bulk RNA-sequencing gene expression profiles of SSCs from fracture calluses of ‘2-mo’, ‘12-mo’, and ‘24-mo’ mice supported our transcriptomic analyses of homeostatic SSCs, including the age-dependent upregulation of pro-hematopoietic and pro-myeloid factors and downregulation of pro-skeletogenic genes (FIG. 10g). Specifically, the expression of Csf1 remained high in 24-mo fracture-derived SSCs (FIG. 3m).

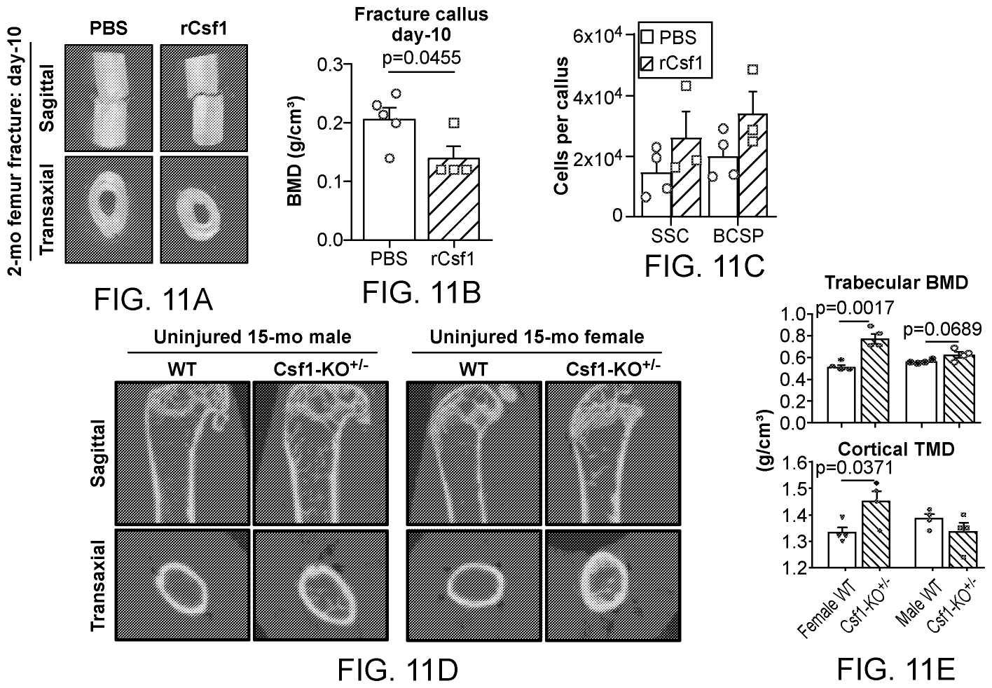

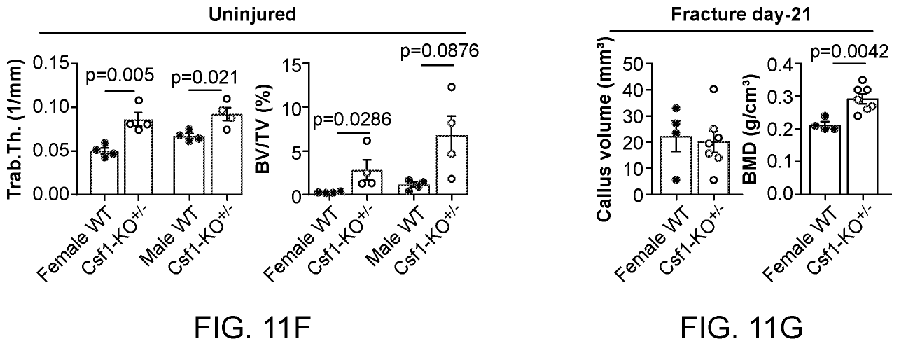

We also tested whether these increases in Csf1 affected bone maintenance and regeneration. We applied hydrogels containing recombinant Csf1 with defined release kinetics to femoral fractures of ‘2-mo’ mice and found impaired healing without alteration of SSC frequency, as measured by μCT and flow cytometry (FIG. 3n & FIG. 11a-c). We also found that 15-month-old haploinsufficient Csf1 mice had femora with increased mechanical strength and higher BMD, bone volume, and trabecular thickness, independent of sex (FIG. 3o & FIG. 11d-f). Unexpectedly, however, femora harvested from transgenic mice at post-fracture day-21 had decreased mechanical strength compared to wild-type controls despite having increased BMD and similar callus volume (FIG. 3p-q & FIG. 11g). This resembled a state of osteopetrosis leading to higher skeletal fragility and suggested that tightly controlled Csf1 levels were necessary to support healthy bone remodeling. In total, SSC lineage derived Csf1 might drive increased bone resorption by stimulating higher osteoclast activity, and this activity needs to be maintained and finely balanced for proper bone health.

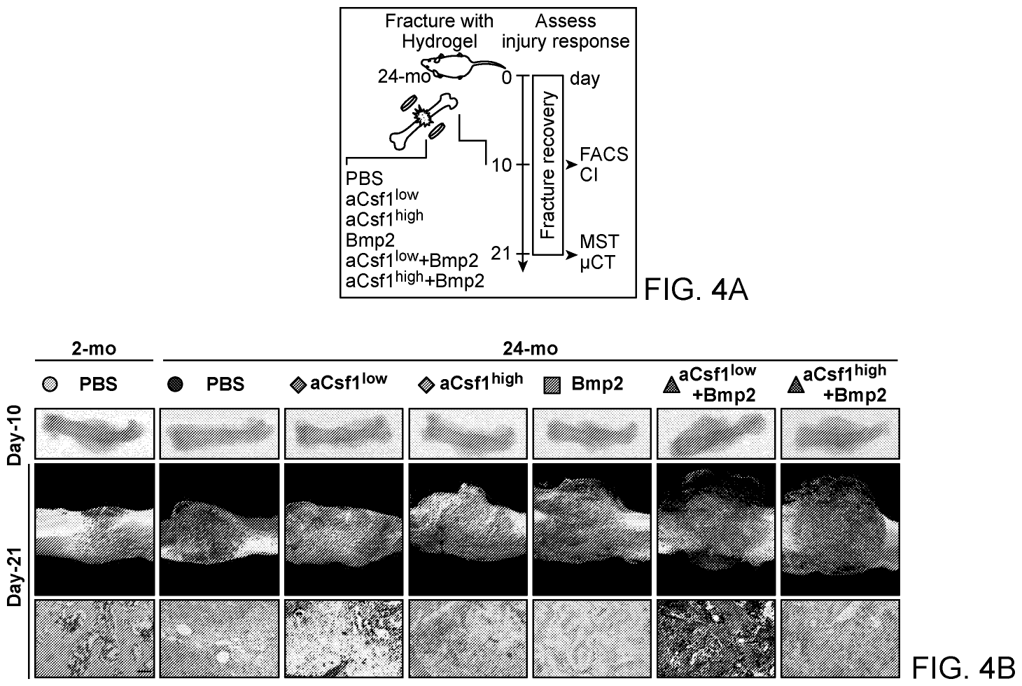

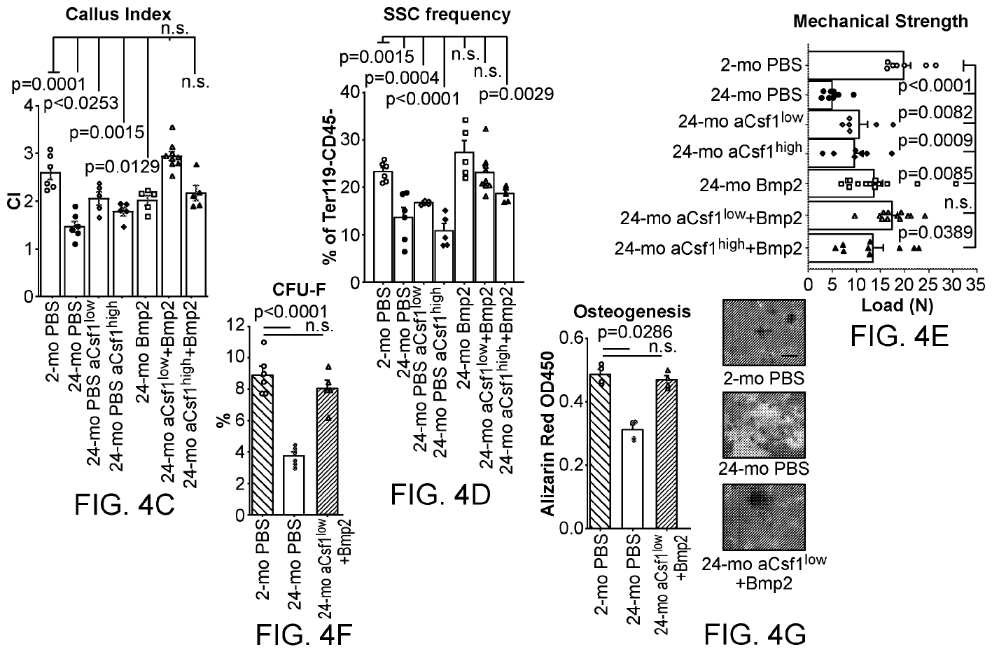

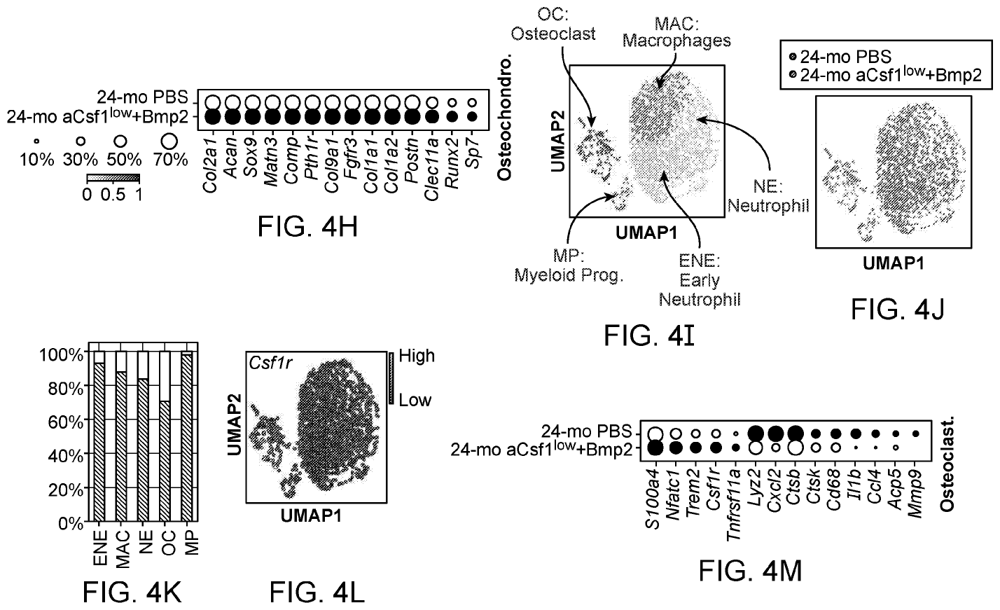

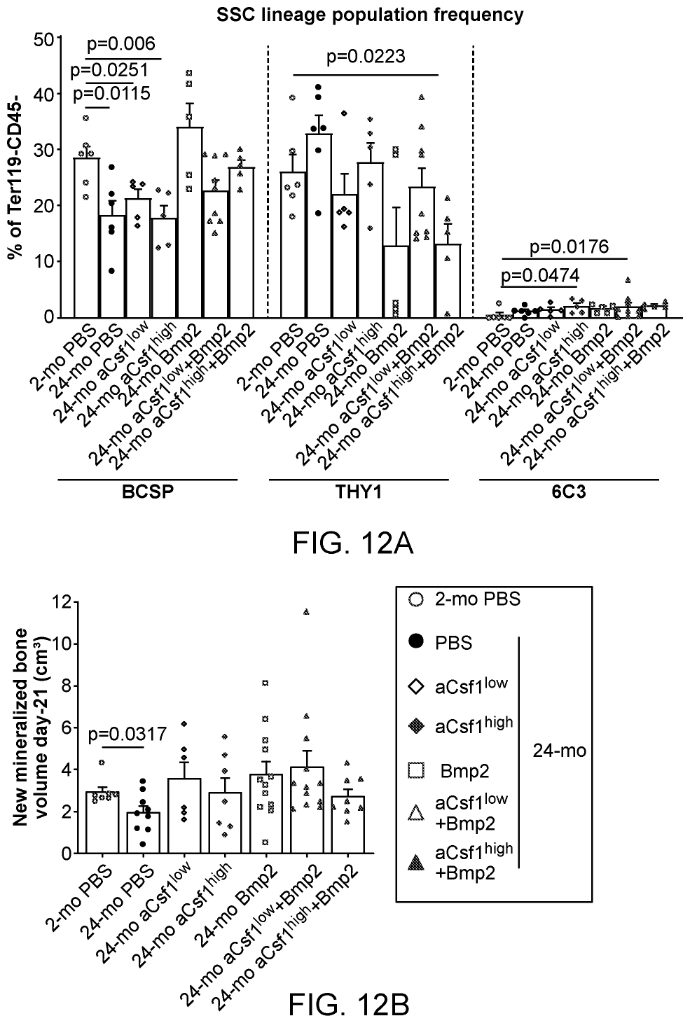

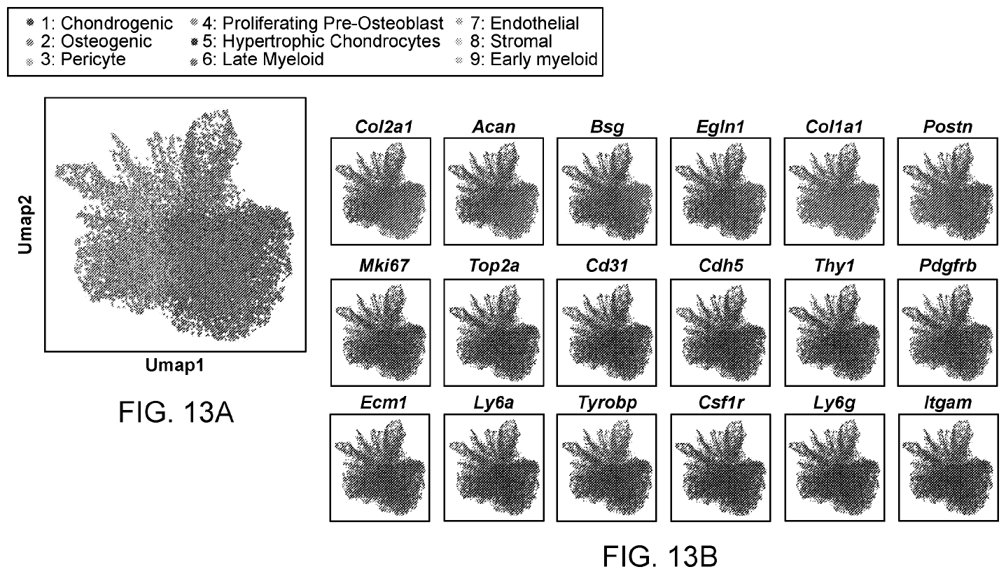

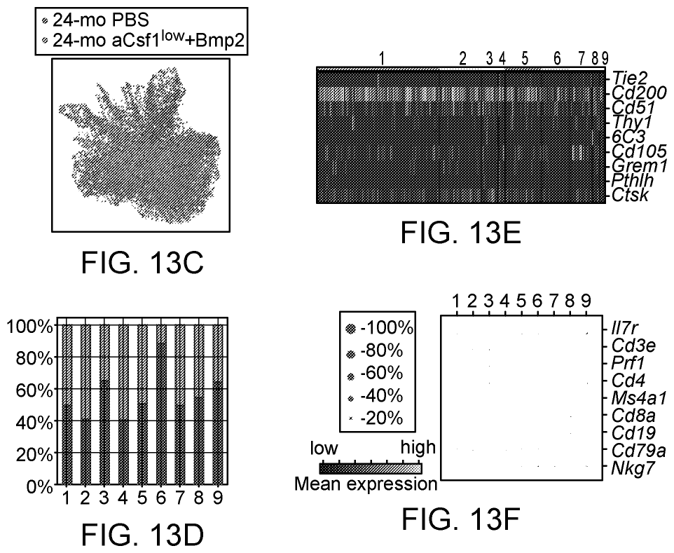

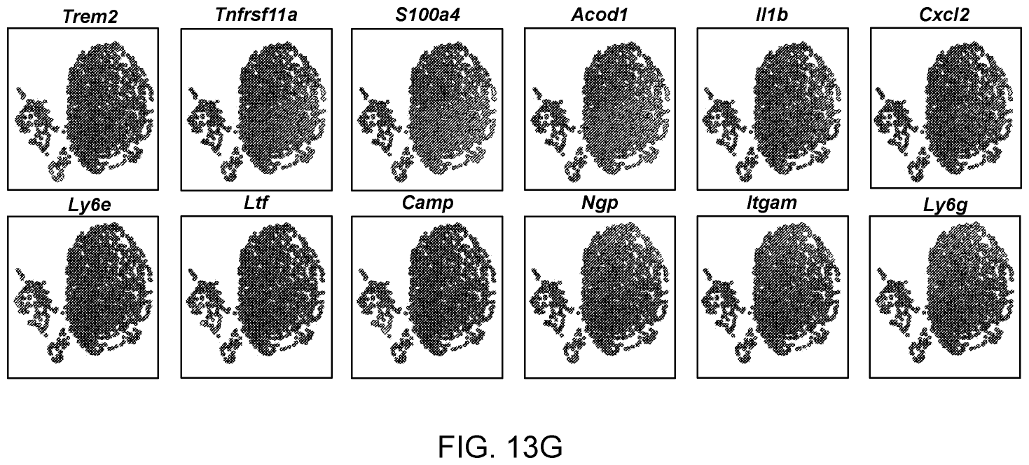

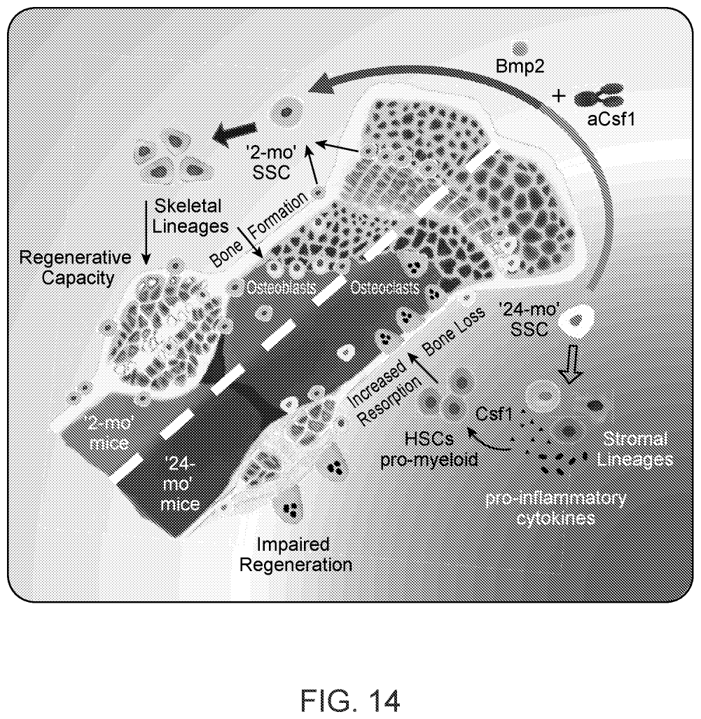

Combinatorial targeting of the aged SSC differentiation defect and pro-resorptive signaling restores youthful bone regeneration. Our findings indicate that aged SSCs lose osteochondrogenic activity but also enhance bone resorption through the generation of an inflammatory, pro-osteoclastic niche. We hypothesized that bone regeneration could be restored in aged mice by targeting each of these pathways locally. To test this hypothesis, we created hydrogels containing the potent osteo-inductive agent Bmp2 (5 μg), a Csf1 antagonist (“aCsf1”; 2 “low” or 5 “high” μg), a combination of both factors, or PBS. We applied these hydrogels to transverse femoral fractures of ‘24-mo’ mice and assessed healing at post-fracture day-10 and -21 (FIG. 4a). ‘2-mo’ mice receiving PBS-containing hydrogels served as control. Callus index at post-fracture day-10 only remained similar to ‘2-mo’ controls when ‘24-mo’ mice were given combinatorial treatments (FIG. 4b-c). While ‘24-mo’ mice treated with Bmp2 alone produced significantly smaller calluses compared to ‘2-mo’ controls, flow cytometry of these calluses showed that Bmp2 increased SSC and BCSP frequencies to youthful levels. This increase in SSC and BCSP frequency was also specifically seen in the combinatorial treatment group with low aCsf1 (FIG. 4d and FIG. 12a). Nonetheless, at post-fracture day-21 all ‘24-mo’ Bmp2 and aCsf1 treatment groups formed comparable amounts of mineralized tissue as ‘2-mo’ control animals (FIG. 12b). Consistent with our observations that genetic ablation of Csf1 is detrimental to fracture repair (FIG. 3q-r), we found that the combination of low, but not high, aCsf1 and Bmp2 together uniquely rescued the mechanical strength of ‘24-mo’ healing femora to that of young bones (FIG. 4e). Strikingly, SSCs from fracture calluses of ‘24-mo’ mice treated with a combination of low aCsf1 and Bmp2 showed youthful colony-forming and osteogenic capacity, tying rejuvenated stem cell characteristics to improved healing outcomes (FIG. 4f-g). Molecularly, 10× scRNAseq of day-10 fracture calluses from ‘24-mo-aCsf1low+Bmp2’ rescue mice showed much higher expression of osteochondrogenic genes in non-hematopoietic cell types compared to the ‘24-mo-PBS’ group (FIG. 4h & FIG. 13a-c). Compositionally, and in support of the treatment, ‘24-mo-aCsf1low+Bmp2’ rescue fracture calluses had higher bone-forming cell fractions including phenotypic SSCs (clusters 2 & 4), while ‘24-mo-PBS’ controls displayed a higher abundance of immune cells, almost exclusively of myeloid origin (FIG. 13d-f). Within hematopoietic cell-enriched clusters (6 & 9) osteoclasts, macrophages, and neutrophils were dominant as expected for healing skeletal tissue (FIG. 4i-j & FIG. 13g). The ‘24-mo-aCsf1low+Bmp2’ rescue treatment reduced overall myeloid cell prevalence and led to higher expression of pre-osteoclastic genes (Csf1r, Trem2, Rankl/Tnfrsf11a) compared to late osteoclast markers (Ctsk, Cd68, Acp5) in ‘24-mo-PBS’ controls supporting the enhanced inhibition of osteoclast maturation by aCsf1 presence (FIG. 4k-m). Altogether, these data show that the local and temporal application of Bmp2 to stimulate SSC activation combined with low levels of aCsf1 to constrain bone resorption reinstates youthful bone regeneration in aged bones (FIG. 50).

This study addressed the complexity of skeletal aging from a bona fide stem cell perspective. Bone has a concerted relationship with the hematopoietic system, and this interaction is essential to the healthy maintenance of bone, blood, and immune systems. Here, we investigated the changing interactions between skeletal and hematopoietic stem cell lineages that occur as a result of aging. First, we show that aged mice maintain phenotypic SSCs that are identified by the same surface markers as in young mice. Second, we demonstrate that bone aging is rooted in cell-intrinsic alterations at the SSC level that ultimately skew skeletal and hematopoietic lineage output, diminish bone integrity, and limit healing capacity. Lastly, targeting SSC autonomous and non-autonomous activities can restore aging-impaired regeneration by simultaneously enhancing local SSC lineage activity while finely modulating of pro-osteoclast activity.

To date, the best studied example of stem cell aging is the HSC system. Our detailed investigation of aged SSCs revealed several interesting comparisons between these stem cell compartments. Whereas HSCs are known to expand with age, we demonstrate that the frequency of SSCs suffers an age-dependent decline. This exhaustion of the SSC pool could deteriorate the skeleton and depress its regenerative capacity on its own accord. However, scRNAseq data indicate that the SSC population loses transcriptomic diversity with age, suggesting that clonal selection of a functionally distinct subpopulation of SSCs could also contribute to the aged skeletal phenotype.

The precedent for the model of age-dependent “clonal skeletogenesis” is set by HSCs, where clonal expansion of lineage-skewed subpopulations is known to affect hematopoietic function. Several studies of aged HSCs demonstrate that cell-intrinsic mechanisms underlie these functional limitations. Interestingly, however, our findings indicate that the aged SSC favor the selective expansion of myeloid-skewed lineage populations both through its own bias towards Thy1+ pro-myeloid stroma and through the upregulation and secretion of pro-myeloid cytokines, a trait it seems to pass on to all its progenies. Ultimately, these shifts toward myeloid-supportive signaling create an altered hematopoietic system increasing osteoclast activity and exacerbating the inflammatory milieu to systemic levels. Therefore, the aged skeletal phenotype is likely a result of an SSC-derived anti-osteogenic and pro-osteoclastic bone marrow microenvironment.

Recent studies propose that SSC and HSC aging could be driven be several other cell-extrinsic factors, such as chronic inflammation, alterations in blood vessel composition, or changes in innervation. In contrast, we showed that aged SSCs consistently underperformed in regard to their osteochondrogenic activity despite changing niche conditions, including renal transplantation into young mice, parabiosis with young mice, and reconstitution of the aged blood system by young HSCs. These experiments indicate that cell-intrinsic mechanisms manifest age-associated SSC properties. While not addressed here, epigenetic priming or drift are other likely drivers of intrinsic SSC aging and should not be disregarded. In addition, because aged SSCs do not give rise to adipocytes despite the well-documented rise of fatty infiltration in aged bone, other unidentified purified stem cell populations likely exist that shape the bone marrow microenvironment.

Notably, we show that intrinsic aged SSC lineage changes also include the upregulation and secretion of inflammatory, pro-osteoclastic factors. Osteoclast maturation and survival is dependent on Rankl and Csf1. Increased expression of Rankl by bone marrow stromal cells has been shown to be a sign of elevated bone marrow adipogenesis which in turn could promote osteoclastogenesis as well. Comparatively, Rankl-KO mice develop osteopetrosis, consistent with our findings in aged haploinsufficient Csf1 mice. Aged stromal cell-derived Csf1 has been previously shown to induce bone loss in vitro; however, our study is the first to show that this phenomenon likely arises through a defined stem cell lineage in vivo. Intriguingly, bone loss induced by increased stromal cell production of Csf1 in ovariectomized mice can be prevented by neutralizing Csf1. These results were not replicated in a fracture setting, as we found that Csf1 antagonism alone could not rescue aged fracture healing.

We also show that aged SSCs are not rejuvenated by systemic factors. Consistent with previous parabiosis studies that found non-diabetic mouse blood does not rescue impaired fracture healing in diabetic mice, exposure to a young circulation did not restore aged SSC function. This finding is in contrast to a prior study that reported a positive effect of heterochronic parabiosis on bone parameters. The results, however, were limited to one post-injury time point and histological analyses. Therefore, it remains doubtful that physiological levels of circulating factors can lead to lasting skeletal rejuvenation, as it has been proposed for other tissues. It seems more reasonable to apply local factors that act on SSC niches to enhance osteochondrogenesis and modulate lineage fates. Accordingly, we show that the combination of recombinant Bmp2 and a low dose of Csf1 antagonist restored aged SSC function, molecular signatures of callus tissue, and fracture healing to levels seen in young mice. Closer examination of molecular changes in these fracture callus tissues support the notion that other cell types aside from SSCs, e.g., osteoblasts and immune cells, are positively affected by the rescue treatment.

### REFERENCES

Boskey, A. L. & Coleman, R. Aging and Bone. J Dent Res 89, 1333-1348 (2010).
Chan, C. K. F. et al. Identification and specification of the mouse skeletal stem cell. Cell 160, 285-298 (2015).
Kaeberlein, M. Lessons on longevity from budding yeast. Nature 464, 513-519 (2010).
Kenyon, C. J. The genetics of ageing. Nature 464, 504-512 (2010).
Rossi, D. J., Jamieson, C. H. M. & Weissman, I. L. Stems cells and the pathways to aging and cancer. Cell 132, 681-696 (2008).
Schmich, J. et al. Induction of reverse development in two marine Hydrozoans. Int. J. Dev. Biol. 51, 45-56 (2007).
Ermolaeva, M., Neri, F., Ori, A. & Rudolph, K. L. Cellular and epigenetic drivers of stem cell ageing. Nature Reviews Molecular Cell Biology 19, 594-610 (2018).
Pang, W. W. et al. Human bone marrow hematopoietic stem cells are increased in frequency and myeloid-biased with age. Proc. Natl. Acad. Sci. U.S.A. 108, 20012-20017 (2011).
Beerman, I. et al. Functionally distinct hematopoietic stem cells modulate hematopoietic lineage potential during aging by a mechanism of clonal expansion. Proc. Natl. Acad. Sci. U.S.A. 107, 5465-5470 (2010).
Geiger, H., de Haan, G. & Florian, M. C. The ageing haematopoietic stem cell compartment. Nat. Rev. Immunol. 13, 376-389 (2013).
Beerman, I. & Rossi, D. J. Epigenetic Control of Stem Cell Potential during Homeostasis, Aging, and Disease. Cell Stem Cell 16, 613-625 (2015).
Ambrosi, T. H., Longaker, M. T. & Chan, C. K. F. A Revised Perspective of Skeletal Stem Cell Biology. Front Cell Dev Biol 7, 189 (2019).
Chan, C. K. F. et al. Identification of the Human Skeletal Stem Cell. Cell 175, 43-56.e21 (2018).
Chan, C. K. F. et al. Clonal precursor of bone, cartilage, and hematopoietic niche stromal cells. PNAS 110, 12643-12648 (2013).
Halloran, B. P. et al. Changes in bone structure and mass with advancing age in the male C57BL/6J mouse. J. Bone Miner. Res. 17, 1044-1050 (2002).
Ferguson, V. L., Ayers, R. A., Bateman, T. A. & Simske, S. J. Bone development and age-related bone loss in male C57BL/6J mice. Bone 33, 387-398 (2003).
Conboy, I. M. & Rando, T. A. Heterochronic parabiosis for the study of the effects of aging on stem cells and their niches. Cell Cycle 11, 2260-2267 (2012).
Marecic, O. et al. Identification and characterization of an injury-induced skeletal progenitor. PNAS 112, 9920-9925 (2015).
Murphy, M. P. et al. Articular cartilage regeneration by activated skeletal stem cells. Nature Medicine 1-10 (2020) doi:10.1038/s41591-020-1013-2.
Mizuhashi, K. et al. Resting zone of the growth plate harbors a unique class of skeletal stem cells. Nature 563, 254-258 (2018).
Debnath, S. et al. Discovery of a periosteal stem cell mediating intramembranous bone formation. Nature 562, 133-139 (2018).
Chan, C. K. F. et al. Endochondral ossification is required for haematopoietic stem-cell niche formation. Nature 457, 490-494 (2009).
Conboy, I. M. et al. Rejuvenation of aged progenitor cells by exposure to a young systemic environment. Nature 433, 760-764 (2005).
Katsimpardi, L. et al. Vascular and neurogenic rejuvenation of the aging mouse brain by young systemic factors. Science 344, 630-634 (2014).
Sinha, M. et al. Restoring systemic GDF11 levels reverses age-related dysfunction in mouse skeletal muscle. Science 344, 649-652 (2014).
Villeda, S. A. et al. Young blood reverses age-related impairments in cognitive function and synaptic plasticity in mice. Nat. Med. 20, 659-663 (2014).
Tevlin, R. et al. Pharmacological rescue of diabetic skeletal stem cell niches. Sci Transl Med 9, (2017).
Pietras, E. M. Inflammation: a key regulator of hematopoietic stem cell fate in health and disease. Blood 130, 1693-1698 (2017).
Wright, D. E., Wagers, A. J., Gulati, A. P., Johnson, F. L. & Weissman, I. L. Physiological migration of hematopoietic stem and progenitor cells. Science 294, 1933-1936 (2001).
Jones, D. L. & Rando, T. A. Emerging models and paradigms for stem cell ageing. Nat. Cell Biol. 13, 506-512 (2011).
Chen, C., Liu, Y., Liu, Y. & Zheng, P. mTOR regulation and therapeutic rejuvenation of aging hematopoietic stem cells. Sci Signal 2, ra75 (2009).
Riddell, J. et al. Reprogramming committed murine blood cells to induced hematopoietic stem cells with defined factors. Cell 157, 549-564 (2014).

O'Flanagan, C. H. et al. Dissociation of solid tumor tissues with cold active protease for single-cell RNA-seq minimizes conserved collagenase-associated stress responses. Genome Biol 20, (2019).
Denisenko, E. et al. Systematic assessment of tissue dissociation and storage biases in single-cell and single-nucleus RNA-seq workflows. Genome Biology 21, 130 (2020).
La Manno, G. et al. RNA velocity of single cells. Nature 560, 494-498 (2018).
Gulati, G. S. et al. Single-cell transcriptional diversity is a hallmark of developmental potential. Science 367, 405-411 (2020).
Xiong, L. et al. Lrp4 in osteoblasts suppresses bone formation and promotes osteoclastogenesis and bone resorption. Proc Natl Acad Sci USA 112, 3487-3492 (2015).
Kong, L. et al. Extracellular matrix protein 1, a direct targeting molecule of parathyroid hormone-related peptide, negatively regulates chondrogenesis and endochondral ossification via associating with progranulin growth factor. FASEB J 30, 2741-2754 (2016).
Menaa, C., Esser, E. & Sprague, S. M. Beta2-microglobulin stimulates osteoclast formation. Kidney Int 73, 1275-1281 (2008).
Suda, T. et al. Modulation of osteoclast differentiation and function by the new members of the tumor necrosis factor receptor and ligand families. Endocr. Rev. 20, 345-357 (1999).
Cenci, S. et al. Estrogen deficiency induces bone loss by enhancing T-cell production of TNF-alpha. J. Clin. Invest. 106, 1229-1237 (2000).
Urist, M. R. Bone: formation by autoinduction. Science 150, 893-899 (1965).
Jaiswal, S. et al. Age-related clonal hematopoiesis associated with adverse outcomes. N. Engl. J. Med. 371, 2488-2498 (2014).
Rossi, D. J. et al. Cell intrinsic alterations underlie hematopoietic stem cell aging. Proc. Natl. Acad. Sci. U.S.A. 102, 9194-9199 (2005).
Rossi, D. J. et al. Deficiencies in DNA damage repair limit the function of haematopoietic stem cells with age. Nature 447, 725-729 (2007).
Josephson, A. M. et al. Age-related inflammation triggers skeletal stem/progenitor cell dysfunction. PNAS 116, 6995-7004 (2019).
Maryanovich, M. et al. Adrenergic nerve degeneration in bone marrow drives aging of the hematopoietic stem cell niche. Nat. Med. 24, 782-791 (2018).
Ho, Y.-H. et al. Remodeling of Bone Marrow Hematopoietic Stem Cell Niches Promotes Myeloid Cell Expansion during Premature or Physiological Aging. Cell Stem Cell 25, 407-418.e6 (2019).
Kusumbe, A. P. et al. Age-dependent modulation of vascular niches for haematopoietic stem cells. Nature 532, 380-384 (2016).
Ambrosi, T. H. et al. Adipocyte Accumulation in the Bone Marrow during Obesity and Aging Impairs Stem Cell-Based Hematopoietic and Bone Regeneration. Cell Stem Cell 20, 771-784.e6 (2017).
Kong, Y. Y. et al. OPGL is a key regulator of osteoclastogenesis, lymphocyte development and lymph-node organogenesis. Nature 397, 315-323 (1999).
Takeshita, S., Fumoto, T., Naoe, Y. & Ikeda, K. Age-related marrow adipogenesis is linked to increased expression of RANKL. J Biol Chem 289, 16699-16710 (2014).
Cao, J. J. et al. Aging increases stromal/osteoblastic cell-induced osteoclastogenesis and alters the osteoclast precursor pool in the mouse. J. Bone Miner. Res. 20, 1659-1668 (2005).
Baht, G. S. et al. Exposure to a youthful circulation rejuvenates bone repair through modulation of β-catenin. Nature Communications 6, 1-10 (2015).
Conese, M., Carbone, A., Beccia, E. & Angiolillo, A. The Fountain of Youth: A Tale of Parabiosis, Stem Cells, and Rejuvenation. Open Med (Wars) 12, 376-383 (2017).
Gulati, G. S. et al. Isolation and functional assessment of mouse skeletal stem cell lineage. Nat Protoc 13, 1294-1309 (2018).
Wilkinson, A. C., Ishida, R., Nakauchi, H. & Yamazaki, S. Long-term ex vivo expansion of mouse hematopoietic stem cells. Nature Protocols 15, 628-648 (2020).
Foster, D. S. et al. Elucidating the fundamental fibrotic processes driving abdominal adhesion formation. Nat Commun 11, 4061 (2020).
Patro, R., Duggal, G., Love, M. I., Irizarry, R. A. & Kingsford, C. Salmon provides fast and bias-aware quantification of transcript expression. Nat Methods 14, 417-419 (2017).
Soneson, C., Love, M. I. & Robinson, M. D. Differential analyses for RNA-seq: transcript-level estimates improve gene-level inferences. F1000Res 4, 1521 (2015).
Jiang, H., Jiang, C., Morgan, D. & Szlufarska, I. Accelerated atomistic simulation study on the stability and mobility of carbon tri-interstitial cluster in cubic SiC. Computational Materials Science 89, 182-188 (2014).
Dobin, A. et al. STAR: ultrafast universal RNA-seq aligner. Bioinformatics 29, 15-21 (2013).
Li, B. & Dewey, C. N. RSEM: accurate transcript quantification from RNA-Seq data with or without a reference genome. BMC Bioinformatics 12, 323 (2011).
Wolf, F. A., Angerer, P. & Theis, F. J. SCANPY: large-scale single-cell gene expression data analysis. Genome Biology 19, 15 (2018).
Nestorowa, S. et al. A single-cell resolution map of mouse hematopoietic stem and progenitor cell differentiation. Blood 128, e20-e31 (2016).
Chen, E. Y. et al. Enrichr: interactive and collaborative HTML5 gene list enrichment analysis tool. BMC Bioinformatics 14, 128 (2013).

### Materials and Methods

Animals: Mice were maintained at the Stanford University Research Animal Facility in accordance with Stanford University guidelines. Animals were given food and water ad libitum and housed in temperature- and light-controlled micro-insulators. Unless otherwise specified, all experiments were conducted using postnatal-day-3 (newborn, 0-mo) and 2-month-old (young, 2-mo) male B6 mice (C57BL/Ka-Thy1.1-CD45.1) from Jackson Laboratories (JAX: 000406) and 24-month-old (aged, 24-mo) male B6 mice from the NIA. Rainbow immunofluorescence experiments were conducted using young and old male Actin-CreERT2; R26VT2/GK3 mice bred and maintained in our laboratory. Rainbow mice were given intraperitoneal injections of 200 mg/kg of tamoxifen (Sigma-Aldrich) dissolved in corn oil 7 and 3 days prior to fracture and 3 days post-fracture. For parabiosis experiments young and old male GFP mice (C57BL/6-Tg(CAG-EGFP) 1Osb/J) from Jackson Laboratories (JAX: 003291) were used. Cells for renal transplants into male NSG mice (NOD.Cg-Prkdcscid Il2rgtm1Wjl/SzJ; JAX: 005557) were also derived from young and old GFP mice. Csf1-KO^{+/-} mice were generated by initial breeding of B6 mice (JAX: 000406) with op/+ mice (B6; C3Fe a/a-Csf1op/J) obtained from Jackson Laboratories (JAX: 000231), followed by op/+B6 mice with op/+ mice interbreeding. Female and male Csf1-KO^{+/-} mice were used in experiments as indicated.

Bi-cortical femoral fracture: Animals were anaesthetized using aerosolized isoflurane and analgesia was administered before incision. The femur was exposed following muscle distraction and lateral dislocation of the patella. A 25-gauge regular bevel needle (BD BioSciences) was inserted between the femoral condyles to provide relative intramedullary fixation, and a transverse fracture was created in the mid-diaphysis using micro-scissors. Then, the patella was relocated, and 6-0 nylon suture (Ethicon) was used to re-approximate the muscles and close the skin. Radiography verified fracture alignment, and animals displaying fracture displacement or intramedullary needle migration were excluded from further analyses.

Hydrogel fabrication and placement: Eight arm polyethylene glycol (PEG) monomers with end groups of norbornene (MW 10 kDa) or mercaptoacetic ester (MW 10 kDa) were dissolved in PBS at a concentration of 20% w/v. Then, photo-initiator lithium phenyl-2,4,6-trimethylbenzoylphosphinate was added to each solution to make a concentration of 0.05% w/v. The two polymer solutions were mixed at a 1:1 volume ratio to obtain a hydrogel precursor solution. Recombinant mouse growth factor BMP2 (FisherScientific, Cat #: 355-BM) and anti-Csf1 (FisherScientific, Cat #MAB4161SP) were added to the precursor solution at desired concentrations. Solutions were exposed to UV (365 nm, 4 mW/cm²) for 5 min in the mold with a volume of 4 μL each to obtain factor-loaded hydrogels in 50% of the concentration noted in figure legends. Two hydrogels were placed at the fracture site, and each was left in place until tissue harvest. One hydrogel was placed anteromedial to the fracture, and the other hydrogel was placed posteromedial to the fracture. PBS-loaded hydrogels served as controls.

X-ray radiography: Femora were harvested and cleaned of soft tissue. The intramedullary pin was removed prior to imaging. Femora were radiographed within 2 hr of harvest using a Lago-X scanner (Spectral Instruments Imaging). Radiograph images were analyzed using ImageJ 1.48v to determine the callus index. The callus index is a ratio of the maximal mid-diaphyseal callus diameter to the maximal diameter of adjacent uninjured diaphysis.

Mechanical strength testing: Mechanical strength testing was conducted using a delaminator maintained by the R.H. Dauskardt laboratory (Stanford, CA). Prior to testing, uninjured and post-fracture day 21 femora were harvested and cleaned of soft tissues. Intramedullary fixation was removed. Femur samples were stored in PBS on ice until testing within 6 hr of harvest. All samples were pre-loaded to a force of 2 Newtons (N) and subsequently underwent a three-point bend test at a compression rate of 2 microns per second. The maximum load (N) sustained prior to fracture was recorded.

Micro-computed tomography: Femora were harvested and cleaned of soft tissue. The intramedullary pin was removed prior to imaging. Femora were scanned within 2 hr of harvest using a Bruker Skyscan 1276 (Bruker Preclinical Imaging) with a source voltage of 85 KV, a source current of 200 μA, a filter setting of Al 1 mm, and pixel size of 12 microns at 2016×1344. Reconstructed samples were analyzed using CT Analyser and CTvox software (Bruker). Trabecular bone parameters of uninjured femur bones were assessed by analyzing a region of 200 sections which was defined 50 sections distal of the end of the growth plate. Fracture calluses were analyzed by selecting 150 sections in both directions of the fracture gap yielding a total area of 300 sections. The exact region spanning fracture callus was then manually selected using CTAn for analyses. Mineralized renal transplants were identified and selected manually with CTAN before analysis.

Dynamic histomorphometric analysis: For calcein labeling of non-decalcified bone specimen mice were intraperitoneally injected with 2.5 mg/ml of calcein (Sigma-Aldrich, Cat #: C-0875) in a 2% sodium bicarbonate solution. Mice were labeled 7-days apart and sacrificed 3-days following the second injection. Following euthanasia, labeled humeri were harvested, fixed in 10% buffered formalin for 48 hours at 4ºC. Specimens were dehydrated up to 100% ethanol prior to embedding in methylmetacrylate (Dorn and Hart Microedge, Inc.). Plastic blocks were trimmed on a grainer/polisher and sectioning was performed on a motorized RM2165 microtome equipped with tungsten-carbide disposable blades. Seven micrometer sections were collected from the bones of the injected mice. A set of consecutive sections located in the same plane between animals was selected for analysis of calcein labeling to quantify dynamic parameters. All calculations were carried out using ImageJ (National Institutes of Health, Bethesda, MD, USA, http://imagej.nih.gov/ij/). Mineralizing surface per bone surface (MS/BS) represents the percentage of bone surface exhibiting mineralizing activity. It is calculated by the formula dL.Pm/BS, where dL.Pm is the double labeled perimeter and BS is the total bone surface per image. Mineral apposition rate (MAR) is the distance between the midpoints of the two labels divided by the time between the midpoints of the interval. Bone formation rate (BFR) is the volume of mineralized bone formed per unit time, it is calculated as the product of MAR and MS/BS.

Histology: Dissected, soft-tissue free specimens were fixed in 2% PFA at 4° C. overnight. Samples were decalcified in 400 mM Ethylenediaminetetraacetic acid (EDTA) in PBS (pH 7.2) at 4ºC for 2 weeks with a change of EDTA twice every week. The specimens were then dehydrated in 30% sucrose at 4° C. overnight. Specimens were embedded in Optimal Cutting Temperature compound (OCT) and sectioned at 5 mm. Representative sections were stained with freshly prepared Hematoxylin and Eosin, Movat Pentachrome, or TRAP staining. Rainbow mouse bone sections were hydrated and mounted for confocal imaging.

Flow cytometry of skeletal progenitors: Skeletal stem cell lineage populations were isolated as previously described. In brief, femora were harvested, cleaned of soft tissue, and crushed using mortar and pestle. Then, the tissue was digested in M199 (ThermoFisher; Cat #: 11150067) with 2.2 mg/ml collagenase Il buffer (Sigma-Aldrich; Cat #: C6885) at 37° C. for 60 minutes. Dissociated cells were strained through a 100-micron nylon filter, washed in staining medium (10% fetal bovine serum [FBS] in PBS), and pelleted at 200 g at 4° C. The cell pellet was resuspended in staining medium and hematopoietic lineage cells were depleted via ACK lysis for 5 minutes. The cells were washed again in staining medium and pelleted at 200 g at 4° C. Then, the cells were prepared for flow cytometry with fluorochrome-conjugated antibodies (ThermoFisher) against CD45 (Cat #: 15-0451), Ter119 (Cat #: 15-5921), CD51 (Cat #: 12-0512), Tie2 (14-5987), Thy1.1 (Cat #: 47-0900), Thy1.2 (Cat #: 47-0902), 6C3 (Cat #: 17-5891), CD49f (Cat #: 11-0495), CD105 (Cat #: 13-1051), and Streptavidin-PE-Cy7 conjugate (Cat #: 25-4317-82). Flow cytometry was conducted on a FACS Aria II Instrument (BD BioSciences) using a 70-micron nozzle in the Shared FACS Facility in the Lokey Stem Cell Institute (Stanford, CA). The skeletal stem cell lineage gating strategy was determined using fluorescence-minus-one controls. Propidium iodide staining was used to determine cell viability. All cell populations were sorted for purity.

Proliferation and apoptosis assays: EdU proliferation assays were conducted according to manufacturer's guidelines. In brief, FACS-purified mSSCs and BCSPs were fixed and permeabilized separately using a fix-perm kit (BD Biosciences; Cat #: 554714) and processed via ClickIT Edu reaction (ThermoFisher; Cat #: C10337). Then, the cells were re-analyzed via flow cytometry for their proliferative potential. For Annexin V apoptosis assays cells were processed using manufacturer's instructions (ThermoFisher; Cat #: V13241) on freshly isolated mSSCs and BCSPs.

Cell culture: Wells were pre-coated with 0.1% gelatin, and cells were cultured in MEM-α with 10% FBS and 1% penicillin-streptomycin (ThermoFisher; Cat #: 15140-122) under low O₂ conditions (2% atmospheric oxygen, 7.5% carbon dioxide). For in vitro colony-forming assays, 500 FACS-purified mSSCs were plated in 6-well tissue culture well plates and maintained in the above conditions for 14 d. Then, colonies were stained using Crystal Violet (Sigma-Aldrich; Cat #: C0775) and quantified under brightfield microscopy. Eligible colonies contained greater than 20 cells. Colony size was determined using ImageJ software of photo-scanned well plates. For in vitro skeletogenic differentiation, 12,000 FACS-purified mSSCs or BCSPs were cultured in separate wells of a 24-well tissue culture plate in the above conditions. When cell confluency in each well reached 80%, the cells were washed in PBS, trypsinized, and transferred to osteogenic differentiation media (10% FBS, 100 μg/ml ascorbic acid, and 10 mM β-glycerophosphate in DMEM for 14 days), chondrogenic media (micromass culture generated by a 5 μl droplet of cell suspension with approx. 1.5×10⁷ cells/ml were pipetted in the center of a 48-well plate and cultured for 2 h in the incubator before adding warm chondrogenic media consisting of DMEMhigh with 10% FBS, 100 nM Dexamethasone, 1 μM L-ascorbic acid-2-phosphate and 10 ng/ml Transforming growth factor β1 and maintaining for 21 days), or adipogenic media (50 μM indomethacin, 1 μM dexamethasone, 0.5 μM isobutylmethylxanthine, 1 nM 3,3′,5-triiodo-L-thyronine (T3) were added for 48 h, followed by further differentiation in growth medium without growth factors and the addition of T3 and insulin only until day 10). At the end of differentiation cells were washed in PBS, fixed in 4% PFA, and stained with Alizarin Red S (Roth; Cat #: A5533-25G) solution (osteogenesis), alcian blue (Alcian Blue 8GX; Cat #: A3157) solution (chondrogenesis), or Oil Red O (Sigma-Aldrich; Cat #: 00625) solution (adipogenesis). The wells were washed with water and imaged under brightfield microscopy. The concentration of Alizarin Red in each well was quantified by lifting the stain with a methanol/acetic acid mixture for 15 min and measuring absorbance at 450 nm using an Ultraspec 2100 UV/Visible Spectrophotometer (Biochrom, Harvard Bioscience). The concentration of Alcian Blue in each well was quantified by measuring absorbance at 595 nm using spectrophotometry. The concentration of Oil Red O was not quantified due to lack of positive staining in investigated cell types. For in vitro cytokine secretion assays, 25,000 FACS purified SSCs or BCSPs were cultured in separate wells of a 24-well tissue culture plate in the above conditions. 48 hours after seeding cells, well plates were washed 3 times with PBS and culture media conditions were changed to serum-free MEM-α with 1% penicillin-streptomycin. After 24 hr, the conditioned media were collected and spun at (200 g) at 4° C. The aspirate was flash frozen and immediately submitted for Luminex analyses at the Human Immune Monitoring Center (Stanford, CA). For osteoclast differentiation assays limbs were dissected out from 8-week old and 24-month old mice. Bones were crushed using a pestle and mortar. Cells were strained through a 70-μm cell strainer prior to being layered on Histopaque-1077 (Sigma-Aldrich; Cat #: 10771) for gradient separation of red blood cells. The cellular interphase was aspirated out, washed with PBS, and centrifuged prior to cell counting using a hemocytometer. Cells were plated in 24-well plates at a density of 200,000 cells per well with MEMα without phenol red, 1% GlutaMAX supplement (Gibco; Cat #: 35050061), 10% Fetal Bovine Serum (FBS), 1% Penicillin-Streptomycin 10,000 U/ml 10⁻⁷ UM Prostaglandin E2 (PGE2) (Sigma-Aldrich; Cat #: P5640), 10 ng/ml Csf1 Recombinant Human Protein (Peprotech; Cat #: 315-02) for 3 days. On day three, media was changed daily to also include 10 ng/ml recombinant mouse RANKL (Peprotech; Cat #: 315-11). Osteoclast culture continued for 7-10 days until large, multinucleated osteoclasts appeared. Cultures were stopped by aspirating off the media and fixing the cells in the culture plate using a fixative solution containing 25 ml Citrate solution, 65 ml acetone and 8 ml 3 7% formaldehyde, as per the Tartrate-Resistant Acid Phosphatase (TRAP) staining protocol (Sigma-Aldrich). Plates were stained for osteoclasts using the TRAP kit (Sigma-Aldrich; Cat #: 387A). Images were acquired at 10× magnification and osteoclast number, cell size, and nuclei count were assessed using ImageJ (NIH, Bethesda, MD).

Renal capsule transplantation: Renal capsule transplantations were conducted as previously described (Chan et al., Cell 2015). In brief, mice were anaesthetized using aerosolized isoflurane and analgesia was administered. In each mouse, a 5 mm dorsal incision was made, and the kidney was exposed manually. Then, a 2 mm incision was created in the renal capsule using a needle bevel, and 3,000 FACS-purified mSSCs or BCSPs resuspended in 2 μl of Matrigel were transplanted beneath the capsule. The kidney was re-approximated manually and incisions were closed using sutures and staples. Grafts were harvested after 28 days for micro-computed tomography and histological analyses.

Parabiosis: Mice were paired 4 weeks prior to experimental intervention in the following chimeric pairs: isochronic young (2×2-month old), heterochronic (1×2-month old and 1×24-month old), and isochronic aged (2×24-month old). Mice were anaesthetized using aerosolized isoflurane and analgesia was administered. An incision from the distal foreleg to the distal hindleg was made on the right side of one parabiont and on the left side of the second parabiont. The forelegs and hindlegs and the dorsal-dorsal and ventral-ventral skin folds were sutured together using 5-0 nylon suture (Ethicon). Flow cytometry verified blood chimerism after 2 weeks via peripheral blood sample. Peripheral blood chimerism of 1:1 was used to determine full fusion of the circulatory systems.

Fractures in parabionts were induced on opposite sides of the pairing flanks as described above. Fractured bones and contralateral uninjured bones were used for histology, flow cytometry, and microCT analyses.

Hematopoietic stem cell transplantation from parabionts: Isochronic and heterochronic parabiotic pairs were created as described above. Parabiotic pairs of 4 weeks were sacrificed, the skeletons of each mouse were harvested, cleaned of soft tissue, and mechanically crushed using mortar and pestle. The bone marrow was harvested, washed in staining buffer, and strained through a 70-micron filter. Then, the bone marrow was spun at 200 g at 4° C. for 5 min. The cell pellet was resuspended in 1 mL of staining buffer, and red blood cells were depleted using ACK lysis. Long-term HSCs (LT-HSCs; Lin ckit+Sca1+CD150+CD34) were isolated by FACS according to their specific surface marker profiles: [Lineage negative (CD3 (Cat #: 15-0031), CD4 (Cat #: 15-0041), CD8 (Cat #: 15-0081), B220 (Cat #: 15-0452), Gr-1 (Cat #: 15-9668), Mac1 (Cat #: 15-0112), Ter119 (Cat #: 15-5921)], positive for c-kit (Cat #: 17-1171), Sca1 (Biolegend; Cat #: 108120), Slam/CD150 (Cat #: 12-1502), and negative for CD34 (Cat #:50-0341). For HSC transplantation, 100 double-sorted GFP+LT-HSCs were combined with 300,000 unsorted host bone marrow cells as helper marrow and injected retro-orbitally into lethally irradiated (800 rad) young mice. For reconstitution analysis mice were analyzed 8 weeks following HSC transplantation to monitor contribution by donor-marked peripheral blood for lymphoid and myeloid lineages. Using a heat lamp, the tail was heated, and a sharp incision was made to collect approximately 4-6 drops of blood in a tube containing 10 mM EDTA/PBS. An equal volume of 2% Dextran/PBS was added to generate a density gradient and incubated at 37 ºC for 30 min to precipitate red blood cells. The supernatant was collected, and the remaining red blood cells were lysed in ACK lysis buffer. Cells were then stained with antibodies (ThermoFisher) for Ter119 (Cat #: 15-5921), B220 (Cat #: 47-0452), CD3 (Cat #: 17-0031), and Mac1 (Cat #: 25-0112). Flow cytometry was conducted on a FACS Aria Il Instrument (BD BioSciences) using a 70-micron nozzle in the Shared FACS Facility in the Lokey Stem Cell Institute (Stanford, CA).

Hematopoietic stem cell transplantation into young and aged mice: For HSC transplantation experiments into 2-month or 24-month-old recipients 100 E15 fetal liver HSCs or 24-month old bone marrow HSCs were injected retro-orbitally into lethally irradiated (800 rad) B6 mice together with 300,000 unsorted host bone marrow cells as helper marrow. Eight weeks after injection bone mineral density was measured by microCT. Additionally, at this timepoint bi-cortical fractures were generated in these mice and regeneration parameters determined at day 10 and 21 after injury.

For HSC transplantation experiments into 2-month or 24-month old recipients 1,000 HSCs from 2-month old GFP-mouse bone marrow were injected retro-orbitally into lethally irradiated (950 rad) B6 mice together with 500,000 Sca1-depleted, unlabeled bone marrow cells as helper marrow. Peripheral blood analysis was conducted at six and 12 weeks after injection. Mice were sacrificed at 12 week timepoint and bone marrow was analyzed for donor-derived (GFP+) hematopoietic lineage tree cell populations as described in Rossi et al.⁴⁴.

Co-culture of SSCs and HSCs: For co-culture experiments SSCs were freshly isolated from 2-month or 24-month-old mice as described above and seeded at 3,000 cells per well into 96-well plates. Cells were maintained in growth media for three to five days until they reached confluency. SSC growth media was removed and replaced with HSC supporting media as described by Wilkinson et al.⁵⁷ supplemented with recombinant Fibroblast growth factor. Briefly, the media contained of 1× Ham's F-12 Nutrient Mix liquid medium (Gibco, Cat #: 11765-054), 1× Penicillin-streptomycin-glutamine (Gibco, Cat #: 10378-016), 1× Insulin-transferrin-selenium-ethanolamine (ITSX; Gibco, Cat #: 51500-056), 1% Polyvinyl alcohol (PVA; 87-90%-hydrolyzed; Sigma, Cat #: P8136), 10 mM HEPES (Gibco, Cat #: 15630-080), 10 ng/ml recombinant animal-free murine stem cell factor (SCF; Peprotech, Cat #: AF-250-03), 5 ng/ml recombinant animal-free murine thrombopoietin (TPO; Peprotech, Cat #: AF-315-14), 25 ng/ml recombinant animal-free murine FGF-basic (bFGF; Peprotech, Cat #: AF-450-33). At this time, 1,000 HSCs from GFP-reporter mice were freshly sorted into each well. Half the media was replenished three days later, and cells were lifted with 0.2% Collagenase at day 6 for FACS analysis or retro-orbital transplantation into lethally irradiated (950 rad) 2-month-old recipient mice (together with 300,000 unlabeled helper marrow cells). Wells with HSCs expanded alone for 6-days were included as control. Peripheral blood analysis was conducted at six and 12 weeks after injection. Mice were sacrificed at 12 week timepoint and bone marrow was analyzed for donor-derived (GFP+) hematopoietic lineage tree cell populations as described in Rossi et al⁴⁴.

Microarray: Microarray analyses were performed on FACS-purified mouse skeletal stem cell as well as hematopoietic lineage populations. Cell suspension from 3-5 different mice were pooled before FACS purification. 5,000-1,0000 cells of target cell populations were directly sorted into tubes containing 1 mL of Trizol. RNA was isolated with RNeasy Micro Kit (Qiagen; Cat #: 74004) as per manufacturer's guidelines. RNA was amplified twice with a Arcturus™ RiboAmp™ PLUS amplification kit (ThermoFisher; Cat #: KIT0521). Amplified cRNA was streptavidin-labeled, fragmented, and hybridized to Affymetrix 430-2.0 arrays (Affymetrix). Arrays were scanned with a Gene Chip Scanner 3000 (Affymetrix) running GCOS 1.1.1 software. Raw microarray data were submitted to Gene Expression Commons (http://gexc.riken.jp) where data normalization was computed against the Common Reference, a large collection (n=11,939) of publicly available microarray data from the National Center for Biotechnology Information Gene Expression Omnibus. Meta-analysis of the Common Reference also provides the dynamic range of each probe set on the array. Only gene probesets with dynamic ranges>6 were selected and in situations where multiple probesets for a gene exists, the probeset with the widest dynamic range was used. Heatmaps representing percentage of gene expression were generated in the Gene Expression Commons.

Bulk RNA sequencing: 10,000 SSCs were freshly FACS-purified from 10-day-old fracture calluses of young (2 mo), middle-aged (12 mo), and aged (24 mo) B6 mice (n=3 each). Bulk mRNA was processed and sequenced as described before. Briefly, RNA was isolated with Qiagen miRNeasy kit (Cat. 1071023), cDNA was prepared using the Clontech Smarter Ultra Low Input RNA kit (Takara Bio, Cat. 634848), and libraries were generated with the Clontech Low Input Library Prep kit (Takara Bio, Cat. 634947). The barcoded samples were pooled and then sequenced on four lanes of NextSeq 500 (Illumina) to obtain 2×76 base pair paired-end reads (average no. of mapped reads per sample: ˜45 million).

Bulk mRNA sequencing data was analyzed as described before. Briefly, raw FASTQ reads from each lane were merged and then aligned to the GENCODE vM20 mouse reference transcripts (GRCm38.p6) with Salmon v0.12.0 using the --seqBias, --gcBias, --posBias, --useVBOpt, --rangeFactorizationBins 4, and --validateMappings flags and otherwise default parameters for paired-end mapping. Salmon results were merged into a single gene-level transcripts per million (TPM) matrix using the R package, tximport v1.10.1 and log 2-normalized. Raw and processed data is available from GEO with Accession GSE166441 (Reviewer token: slmbusuclrstrur).

Smart-Seq2 Single-cell RNA-sequencing: Single cells were isolated via FACS as described above from freshly processed long bones pooled from 3-5 mice of either postnatal day-3 (newborn), 2-month-old, or 24-month-old B6 mice. Single cells were captured in separate wells of a 96-well plate containing 4 μl lysis buffer containing 1 U/μL RNase inhibitor (Clontech, Cat #: 2313B), 0.1% Triton (Thermo Fisher Scientific, Cat #: 85111), 2.5 mM dNTP (Invitrogen, Cat #: 10297-018), 2.5 UM oligo dT30VN (IDT, custom: 5′-AAGCAGTGGTATCAACGCAGAGTACT30VN-3′), and 1:600,000 ERCC (External RNA Controls Consortium) ExFold RNA Spike-In Mix 2 (ERCC; Invitrogen, Cat #: 4456739)) in nuclease-free water (Thermo Fisher Scientific, Cat #: 10977023). Cells were spun down and plates kept at −80° C. until cDNA synthesis. cDNA from single cell RNA was performed using oligo-dT primed reverse transcription with SMARTScribe reverse transcriptase (Clontech, Cat #: 639538) and a locked-nucleic acid containing template-switching oligonucleotide (TSO; Exiqon, custom: 5′-AAGCAGTGGTATCAACGCAGAGTACATrGrG+G-3′). PCR amplification was conducted using KAPA HiFi HotStart ReadyMix (Kapa Biosystems, Cat #: KK2602) with ISPCR primers (IDT, custom: 5′-AAGCAGTGGTATCAACGCAGAGT-3′). Amplified cDNA was then purified using 0.6× volume of Agencourt AMPure XP beads (Beckman Coulter, Cat #: A63882). 12-24 random wells per 96-well plate containing cDNA were quantified for concentration and size distribution on a capillary electrophoresis-based, high-sensitivity AATI 96-capillary fragment analyzer (Advanced Analytical, Agilent HS NGS Fragment Kit [1-6000 bp]). Concentration of wells containing single cell cDNA was averaged to determine a dilution factor used to normalize each well to the desired concentration range (0.05-0.16 ng/μL). The content of four 96 well plates was then consolidated into 384-well plates without cherry-picking/removing wells not holding any single cell cDNA. Subsequently the 384-well plates were used for library preparation (Nextera XT kit; Illumina, Cat #: FC-131-1096) using a semi-automated pipeline. The barcoded libraries of each well were pooled, cleaned-up, and size-selected using two rounds (0.35× and 0.75×) of Agencourt AMPure XP beads (Beckman Coulter), as recommended by the Nextera XT protocol (Illumina). A high-sensitivity AATI 96-capillary fragment analyzer run was used to assess fragment distribution and concentrations. Pooled libraries were sequenced on a NovaSeq6000 (Illumina) to obtain 2×101 bp paired-end reads.

Single-cell data processing and analysis: Single RNA-sequencing data was demultiplexed using bcl2fastq2 2.18 (Illumina). Raw reads were further processed using skewer for 3′ quality-trimming, 3′ adaptor-trimming, and removal of degenerate reads. Trimmed reads were then mapped to the mouse genome vM20 using STAR 2.442 (with an average of >70% of uniquely mapped reads), and counts per million (CPM) was calculated using RSEM 1.2.2163. Data was explored and plots were generated using the Scanpy package (version 1.8.0.)⁶⁴. To select for high quality single cells, cells with less than 250 genes and less than 2,500 counts were excluded. Genes that were detectable in less than 3 cells across all cells were also removed. Additionally, any cell exceeding 7.5% mitochondrial and 5% ribosomal gene content as well as with a ERCC fraction higher than 30% were excluded. After these stringent quality filtering steps 65 ‘0-mo’ SSCs, 170 ‘2-mo’ SSCs, and 67 ‘24-mo’ SSCs were used for downstream analysis. Raw CPM values were mean- and log-normalized. Batch correction using Scanpy integrated ComBat was applied for cells derived from separate 96-well processing plates followed by data scaling. Clustering of single cell data was done using UMAP (v. 0.4.6.) and leidenalg (v. 0.8.2.). Principal component (PC) elbow plots were used to select number of PCs for each clustering analysis. Genes were ranked and differential expression between groups was assessed by Wilcoxon rank-sum test (Mann-Whitney-U) for identification of cluster-specific genes. Cell cycle status was assessed using the ‘score_genes_cell_cycle’ function with the updated gene list provided by Nestorowa et al. Lineage trajectory inference was performed using RNAvelocity³⁵. Differentially expressed genes between ‘24-mo’ and ‘0-mo’/′2-mo′ groups were tested for gene ontology enrichment using the Enrichr⁶⁶ webserver version of GO Biological Processes. Only significantly enriched GO terms were considered (adj. p<0.1) and provided combined statistical scores are displayed. Data is available with GEO Accession GSE161946 (Reviewer token: apqlussmhrodjkx).

10× Genomics scRNAseq: Fracture callus tissue was harvested from 24-mo mice at day-10 after injury. Three fracture calluses from PBS-control and aCsf^{low}/BMP2-treated mice each were processed, digested, and prepared for FACS as described above. Single cell solutions of each treatment group were then pooled (n=3 per group) and 2×10⁵ PI-Ter119− cells were sorted into collection tubes containing FACS buffer. Cells were then processed with 10× Chromium Next GEM Single Cell 3′ GEM kit (10× Genomics Inc, v3.1) according to manufacturer's instruction to target 10,000 cells per group. Barcoded samples were demultiplexed, aligned to the mouse genome (vM20), and UMI-collapsed with the Cellranger toolkit with standard settings and --force-cells=10,000 (version 4.0.0, 10× Genomics Inc) sequenced on an Illumina NextSeq500 platform yielding an average of 80,589 reads per cell and a median of 1,633 genes per cells for the PBS-control group and 100,053 reads per cell and a median of 2,062 genes per cells for the aCsf^{low}/BMP2-treated group. We used the Scanpy package (version 1.7.1.)⁶⁴ to explore the data. First, quality filtering to exclude multiplets and cells of poor quality was conducted by only keeping cells with a gene count of >250 and <3000 with less than 10% mitochondrial and 20% ribosomal gene content leaving 17,230 cells. Genes expressed in less than 3 cells across all cells were also removed from downstream analysis. Data was log-normalized, batch corrected (for treatment groups) with ComBat implementation and scaled for analysis. Dimensionality reduction and Leiden clustering as well as subclustering were conducted choosing parameters based on PCA elbow plot. All further analyses were conducted as described for SmartSeq2 above. Data is available with GEO Accession GSE172149 (Reviewer token: obipiyoqltstfel).

Statistical analyses: Statistical significance between two groups was determined using two-tailed, unpaired Student's t-test unless stated otherwise in the figure legend. Normality was assessed by Shapiro-Wilk test. If normality was not met Mann-Whitney test was conducted. If data showed unequal variances (F-test) the t-test was adjusted with Welch's correction. For comparison of more than two groups ANOVA analysis was used with appropriate posthoc test as described in the figure legends. P-values were considered significant if p<0.05. Statistical analyses were performed using GraphPad Prism 9 (GraphPad). All data points represent biological replicates, unless indicated otherwise in the figure legend. The exact sample size for each experiment is included in the figure legends. No statistical method pre-determined sample size. All data are presented as mean+standard error of the mean (SEM) unless otherwise stated in figure legend.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of the present invention is embodied by the appended claims.

1. A method for improving bone regeneration in an aged mammal, the method comprising:
contacting skeletal stem cells with a combination of factors that reactivate aged SSCs and concurrently abate crosstalk to hematopoietic cells favoring an inflammatory milieu.

2. The method of claim 1, wherein the combination of factors is a co-formulated composition comprising an effective dose of a BMP2 activating agent and an inhibitor of CSF1.

3. The method of claim 2, wherein the BMP2 activating agent and CSF1 inhibitor are implanted in a drug delivery device.

4. The method of claim 3, wherein the drug delivery device comprises as the sole active agents a BMP2 activating agent and a CSF1 inhibitor.

5. The method of claim 2, wherein the BMP2 activating agent is human BMP2 protein provided at a unit dose of from about 50 μg to about 10 mg.

6-8. (canceled)

9. The method of claim 2, wherein the CSF1 inhibitor is an antibody specific for CSF1 or CSF1R provided at a dose of from about 20 μg to about 5 mg.

10-11. (canceled)

12. The method of claim 2, wherein the implant is a biodegradable implant.

13. The method of claim 12, wherein the biodegradable implant is a block polymer implant comprising poly(caprolactone) (PCL), poly(lactic acid) (PLA) or poly(lactic-co-glycolic acid) (PLGA).

14. (canceled)

15. The method of claim 12, wherein the biodegradable implant comprises collagen, hyaluronic acid, cellulose, chitosan, silk, gelatin, albumin, elastin or milk proteins.

16. The method of claim 12 wherein the biodegradable implant is a hydrogel.

17. The method of claim 2, wherein the drug delivery device is implanted at the site of local acute injury in the absence of exogenous cells.

18. The method of claim 2, wherein the implant is provided immediately or within 3 days following a local acute injury.

19. (canceled)

20. A drug delivery device comprising an effective dose of a BMP2 activating agent and an inhibitor of CSF1 for reactivation of aged skeletal stem cells.

21. The device of claim 20, wherein the drug delivery device comprises as the sole active agents a BMP2 activating agent and a CSF1 inhibitor.

22. The device of claim 20, wherein the BMP2 activating agent is human BMP2 protein at a unit dose of from about 50 μg to about 10 mg.

23-25. (canceled)

26. The device of claim 20, wherein the CSF1 inhibitor is an antibody specific for CSF1 or CSF1R provided at a dose of from about 20 μg to about 5 mg.

27-28. (canceled)

29. The device of claim 20, wherein the device is a biodegradable implant.

30. The device of claim 29, wherein the biodegradable implant is a block polymer implant comprising poly(caprolactone) (PCL), poly(lactic acid) (PLA) or poly(lactic-co-glycolic acid) (PLGA).

31. (canceled)

32. The device of claim 30, wherein the biodegradable implant comprises collagen, hyaluronic acid, cellulose, chitosan, silk, gelatin, albumin, elastin or milk proteins.

33. The device of claim 20 wherein the biodegradable implant is a hydrogel.
